(19) United States
(12) Patent Application Publication
(10) Pub. No.: US 2002/0076414 A1
(43) Pub. Date: Jun. 20, 2002

(54) Compounds for immunotherapy and diagnosis of colon cancer and methods for their use

(75) Inventors: Jiangchun Xu, Bellevue, WA (US); Michael J. Lodes, Seattle, WA (US); Heather Secrist, Seattle, WA (US); Darin R. Benson, Seattle, WA (US); Madeleine Joy Meagher, Seattle, WA (US); John A. Stolk, Bothell, WA (US); Tongtong Wang, Medina, WA (US); Yuqiu Jiang, Kent, WA (US); Carole L. Smith, Seattle, WA (US); Gordon E. King, Shoreline, WA (US); Aijun Wang, Issaquah, WA (US); Jonathan D. Clapper, Seattle, WA (US)

Correspondence Address: SEED INTELLECTUAL PROPERTY LAW GROUP PLLC, 701 FIFTH AVE, SUITE 6300, SEATTLE, WA, 98104-7092, US

(73) Assignee: Corixa Corporation, Seattle, WA (US)

(21) Appl. No.: 09/922,217

(22) Filed: Aug. 3, 2001

Related U.S. Application Data
(63) Continuation-in-part of application No. 09/833,263, filed on Apr. 10, 2001.
Application No. 09/833,263 is a continuation-in-part of application No. 09/649,811, filed on Aug. 28, 2000.
Application No. 09/649,811 is a continuation-in-part of application No. 09/609,448, filed on Jun. 29, 2000.
Application No. 09/609,448 is a continuation-in-part of application No. 09/575,251, filed on May 19, 2000.
Application No. 09/575,251 is a continuation-in-part of application No. 09/519,444, filed on Mar. 6, 2000.
Application No. 09/519,444 is a continuation-in-part of application No. 09/504,629, filed on Feb. 15, 2000.
Application No. 09/504,629 is a continuation-in-part of application No. 09/480,321, filed on Jan. 10, 2000, now abandoned.
Application No. 09/480,321 is a continuation-in-part of application No. 09/476,296, filed on Dec. 30, 1999.
Application No. 09/476,296 is a continuation-in-part of application No. 09/454,150, filed on Dec. 2, 1999, now abandoned.
Application No. 09/454,150 is a continuation-in-part of application No. 09/444,252, filed on Apr. 10, 2000.
Application No. 09/444,252 is a continuation-in-part of application No. 09/401,064, filed on Sep. 22, 1999.
Application No. 09/401,064 is a continuation-in-part of application No. 09/347,496, filed on Jul. 2, 1999.
Application No. 09/347,496 is a continuation-in-part of application No. 09/221,298, filed on Dec. 23, 1998.

(30) Foreign Application Priority Data
Dec. 23, 1999 (WO) PCT/US99/30909

(51) Int. Cl. A61K 39/00; C12Q 1/68; G01N 33/574; C07H 21/04; C12N 9/00; C12N 5/06; C12P 21/02

(52) U.S. Cl. 424/185.1; 435/6; 435/7.23; 435/69.1; 435/183; 435/325; 435/320.1; 536/23.1

(57) ABSTRACT

Compositions and methods for the therapy and diagnosis of cancer, such as colon cancer, are disclosed. Compositions may comprise one or more colon tumor proteins, immunogenic portions thereof, or polynucleotides that encode such portions. Alternatively, a therapeutic composition may comprise an antigen presenting cell that expresses a colon tumor protein, or a T cell that is specific for cells expressing such a protein. Such compositions may be used, for example, for the prevention and treatment of diseases such as colon cancer. Diagnostic methods based on detecting a colon tumor protein, or mRNA encoding such a protein, in a sample are also provided.

### CROSS-REFERENCE TO RELATED APPLICATIONS

This application is a continuation-in-part of U.S. patent application Ser. No. 09/833,263 filed Apr. 10, 2001, which is a continuation-in-part of U.S. patent application Ser. No. 09/649,811 filed Aug. 28, 2000, which is a continuation-in-part of U.S. patent application Ser. No. 09/609,448, filed Jun. 29, 2000, which is a continuation-in-part of U.S. patent application Ser. No. 09/575,251 filed May 19, 2000, which is a continuation-in-part of U.S. patent application Ser. No. 09/519,444, filed Mar. 6, 2000 which is a continuation-in-part of U.S. patent application Ser. No. 09/504,629, filed Feb. 15, 2000, which is a continuation-in-part of U.S. patent application Ser. No. 09/480,321, filed Jan. 10, 2000, which is a continuation-in-part of U.S. patent application Ser. No. 09/476,296, filed Dec. 30, 1999, which is a continuation-in-part of U.S. patent application Ser. No. 09/454,150, filed Dec. 2, 1999, which is a continuation-in-part of U.S. patent application Ser. No. 09/444,252, filed Nov. 19, 1999 which is a continuation-in-part of U.S. patent application Ser. No. 09/401,064, filed Sep. 22, 1999 which is a continuation-in-part of U.S. patent application Ser. No. 09/347,496, filed Jul. 2, 1999, which is a continuation-in-part of U.S. patent application Ser. No. 09/221,298, filed Dec. 23, 1998, each of which applications is co-pending. This application also claims the priority benefit of PCT/US99/30909, filed Dec. 23, 1999, published.

### BACKGROUND OF THE INVENTION

1. Field of the Invention

The present invention relates generally to therapy and diagnosis of cancer, such as colon cancer. The invention is more specifically related to polypeptides comprising at least a portion of a colon tumor protein, and to polynucleotides encoding such polypeptides. Such polypeptides and polynucleotides may be used in vaccines and pharmaceutical compositions for prevention and treatment of colon cancer, and for the diagnosis and monitoring of such cancers.

2. Description of the Related Art

Cancer is a significant health problem throughout the world. Although advances have been made in detection and therapy of cancer, no vaccine or other universally successful method for prevention or treatment is currently available. Current therapies, which are generally based on a combination of chemotherapy or surgery and radiation, continue to prove inadequate in many patients.

Colon cancer is the second most frequently diagnosed malignancy in the United States as well as the second most common cause of cancer death. The five-year survival rate for patients with colorectal cancer detected in an early localized stage is 92%; unfortunately, only 37% of colorectal cancer is diagnosed at this stage. The survival rate drops to 64% if the cancer is allowed to spread to adjacent organs or lymph nodes, and to 7% in patients with distant metastases.

The prognosis of colon cancer is directly related to the degree of penetration of the tumor through the bowel wall and the presence or absence of nodal involvement, consequently, early detection and treatment are especially important. Currently, diagnosis is aided by the use of screening assays for fecal occult blood, sigmoidoscopy, colonoscopy and double contrast barium enemas. Treatment regimens are determined by the type and stage of the cancer, and include surgery, radiation therapy and/or chemotherapy. Recurrence following surgery (the most common form of therapy) is a major problem and is often the ultimate cause of death. In spite of considerable research into therapies for the disease, colon cancer remains difficult to diagnose and treat. In spite of considerable research into therapies for these and other cancers, colon cancer remains difficult to diagnose and treat effectively. Accordingly, there is a need in the art for improved methods for detecting and treating such cancers. The present invention fulfills these needs and further provides other related advantages.

### BRIEF SUMMARY OF THE INVENTION

In one aspect, the present invention provides polynucleotide compositions comprising a sequence selected from the group consisting of:

(a) sequences provided in SEQ ID NOs: 1-121, 123-197, 205-630, 632-684, 686, 690-691, 694-1058, 1069, 1071-1076, 1082, 1084, 1086, 1092, 1094, 1096-1101, 1103-1106, 1111-1114, 1119, and 1120,

(b) complements of the sequences provided in SEQ ID NOs: 1-121, 123-197, 205-630, 632-684, 686, 690-691, 694-1058, 1069, 1071-1076, 1082, 1084, 1086, 1092, 1094, 1096-1101, 1103-1106, 1111-1114, 1119, and 1120;

(c) sequences consisting of at least 20 contiguous residues of a sequence provided in SEQ ID NOs: 1-121, 123-197, 205-630, 632-684, 686, 690-691, 694-1058, 1069, 1071-1076, 1082, 1084, 1086, 1092, 1094, 1096-1101, 1103-1106, 1111-1114, 1119, and 1120;

(d) sequences that hybridize to a sequence provided in SEQ ID NOs: 1-121, 123-197, 205-630, 632-684, 686, 690-691, 694-1058, 1069, 1071-1076, 1082, 1084, 1086, 1092, 1094, 1096-1101, 1103-1106, 1111-1114, 1119, and 1120, under moderately stringent conditions;

(e) sequences having at least 75% identity to a sequence of SEQ ID NOs: 1-121, 123-197, 205-630, 632-684, 686, 690-691, 694-1058, 1069, 1071-1076, 1082, 1084, 1086, 1092, 1094, 1096-1101, 1103-1106, 1111-1114, 1119, and 1120;

(f) sequences having at least 90% identity to a sequence of SEQ ID NOs: 1-121, 123-197, 205-630, 632-684, 686, 690-691, 694-1058, 1069, 1071-1076, 1082, 1084, 1086, 1092, 1094, 1096-1101, 1103-1106, 1111-1114, 1119, and 1120, and

(g) degenerate variants of a sequence provided in SEQ ID NOs: 1-121, 123-197, 205-630, 632-684, 686, 690-691, 694-1058, 1069, 1071-1076, 1082, 1084, 1086, 1092, 1094, 1096-1101, 1103-1106, 1111-1114, 1119, and 1120,

In one preferred embodiment, the polynucleotide compositions of the invention are expressed in at least about 20%, more preferably in at least about 30%, and most preferably in at least about 50% of colon tumors samples tested, at a level that is at least about 2-fold, preferably at least about 5-fold, and most preferably at least about 10-fold higher than that for normal tissues.

The present invention, in another aspect, provides polypeptide compositions comprising an amino acid sequence that is encoded by a polynucleotide sequence described above.

The present invention further provides polypeptide compositions comprising an amino acid sequence selected from the group consisting of sequences recited in SEQ ID NOs: 122, 198-204, 631, 685, 687, 692, 693, 1059-1068, 1070, 1077-1081, 1083, 1085, 1087, 1093, 1095, 1102, 1107-1110, 1115-1118, 1121, and 1122.

In certain preferred embodiments, the polypeptides and/or polynucleotides of the present invention are immunogenic, i.e., they are capable of eliciting an immune response, particularly a humoral and/or cellular immune response, as further described herein.

The present invention further provides fragments, variants and/or derivatives of the disclosed polypeptide and/or polynucleotide sequences, wherein the fragments, variants and/or derivatives preferably have a level of immunogenic activity of at least about 50%, preferably at least about 70% and more preferably at least about 90% of the level of immunogenic activity of a polypeptide sequence set forth in SEQ ID NOs: 122, 198-204, 631, 685, 687, 692, 693, 1059-1068, 1070, 1077-1081, 1083, 1085, 1087, 1093, 1095, 1102, 1107-1110, 1115-1118, 1121, and 1122 or a polypeptide sequence encoded by a polynucleotide sequence set forth in SEQ ID NOs: 1-121, 123-197, 205-630, 632-684, 686, 690-691, 694-1058, 1069, 1071-1076, 1082, 1084, 1086, 1092, 1094, 1096-1101, 1103-1106, 1111-1114, 1119, and 1120,.

The present invention further provides polynucleotides that encode a polypeptide described above, expression vectors comprising such polynucleotides and host cells transformed or transfected with such expression vectors.

Within other aspects, the present invention provides pharmaceutical compositions comprising a polypeptide or polynucleotide as described above and a physiologically acceptable carrier.

Within a related aspect of the present invention, the pharmaceutical compositions, e.g., vaccine compositions, are provided for prophylactic or therapeutic applications. Such compositions generally comprise an immunogenic polypeptide or polynucleotide of the invention and an immunostimulant, such as an adjuvant.

The present invention further provides pharmaceutical compositions that comprise: (a) an antibody or antigen-binding fragment thereof that specifically binds to a polypeptide of the present invention, or a fragment thereof; and (b) a physiologically acceptable carrier.

Within further aspects, the present invention provides pharmaceutical compositions comprising: (a) an antigen presenting cell that expresses a polypeptide as described above and (b) a pharmaceutically acceptable carrier or excipient. Illustrative antigen presenting cells include dendritic cells, macrophages, monocytes, fibroblasts and B cells.

Within related aspects, pharmaceutical compositions are provided that comprise: (a) an antigen presenting cell that expresses a polypeptide as described above and (b) an immunostimulant.

The present invention further provides, in other aspects, fusion proteins that comprise at least one polypeptide as described above, as well as polynucleotides encoding such fusion proteins, typically in the form of pharmaceutical compositions, e.g., vaccine compositions, comprising a physiologically acceptable carrier and/or an immunostimulant. The fusions proteins may comprise multiple immunogenic polypeptides or portions/variants thereof, as described herein, and may further comprise one or more polypeptide segments for facilitating the expression, purification and/or immunogenicity of the polypeptide(s).

Within further aspects, the present invention provides methods for stimulating an immune response in a patient, preferably a T cell response in a human patient, comprising administering a pharmaceutical composition described herein. The patient may be afflicted with colon cancer, in which case the methods provide treatment for the disease, or patient considered at risk for such a disease may be treated prophylactically.

Within further aspects, the present invention provides methods for inhibiting the development of a cancer in a patient, comprising administering to a patient a pharmaceutical composition as recited above. The patient may be afflicted with colon cancer, in which case the methods provide treatment for the disease, or patient considered at risk for such a disease may be treated prophylactically.

The present invention further provides, within other aspects, methods for removing tumor cells from a biological sample, comprising contacting a biological sample with T cells that specifically react with a polypeptide of the present invention, wherein the step of contacting is performed under conditions and for a time sufficient to permit the removal of cells expressing the protein from the sample.

Within related aspects, methods are provided for inhibiting the development of a cancer in a patient, comprising administering to a patient a biological sample treated as described above.

Methods are further provided, within other aspects, for stimulating and/or expanding T cells specific for a polypeptide of the present invention, comprising contacting T cells with one or more of: (i) a polypeptide as described above; (ii) a polynucleotide encoding such a polypeptide; and/or (iii) an antigen presenting cell that expresses such a polypeptide; under conditions and for a time sufficient to permit the stimulation and/or expansion of T cells. Isolated T cell populations comprising T cells prepared as described above are also provided.

Within further aspects, the present invention provides methods for inhibiting the development of a cancer in a patient, comprising administering to a patient an effective amount of a T cell population as described above.

The present invention further provides methods for inhibiting the development of a cancer in a patient, comprising the steps of: (a) incubating CD4⁺ and/or CD8⁺ T cells isolated from a patient with one or more of: (i) a polypeptide comprising at least an immunogenic portion of polypeptide disclosed herein; (ii) a polynucleotide encoding such a polypeptide; and (iii) an antigen-presenting cell that expressed such a polypeptide; and (b) administering to the patient an effective amount of the proliferated T cells, and thereby inhibiting the development of a cancer in the patient. Proliferated cells may, but need not, be cloned prior to administration to the patient.

Within further aspects, the present invention provides methods for determining the presence or absence of a cancer, preferably a colon cancer, in a patient comprising: (a) contacting a biological sample obtained from a patient with a binding agent that binds to a polypeptide as recited above; (b) detecting in the sample an amount of polypeptide that binds to the binding agent; and (c) comparing the amount of polypeptide with a predetermined cut-off value, and therefrom determining the presence or absence of a cancer in the patient. Within preferred embodiments, the binding agent is an antibody, more preferably a monoclonal antibody.

The present invention also provides, within other aspects, methods for monitoring the progression of a cancer in a patient. Such methods comprise the steps of: (a) contacting a biological sample obtained from a patient at a first point in time with a binding agent that binds to a polypeptide as recited above; (b) detecting in the sample an amount of polypeptide that binds to the binding agent; (c) repeating steps (a) and (b) using a biological sample obtained from the patient at a subsequent point in time; and (d) comparing the amount of polypeptide detected in step (c) with the amount detected in step (b) and therefrom monitoring the progression of the cancer in the patient.

The present invention further provides, within other aspects, methods for determining the presence or absence of a cancer in a patient, comprising the steps of: (a) contacting a biological sample obtained from a patient with an oligonucleotide that hybridizes to a polynucleotide that encodes a polypeptide of the present invention; (b) detecting in the sample a level of a polynucleotide, preferably mRNA, that hybridizes to the oligonucleotide; and (c) comparing the level of polynucleotide that hybridizes to the oligonucleotide with a predetermined cut-off value, and therefrom determining the presence or absence of a cancer in the patient. Within certain embodiments, the amount of mRNA is detected via polymerase chain reaction using, for example, at least one oligonucleotide primer that hybridizes to a polynucleotide encoding a polypeptide as recited above, or a complement of such a polynucleotide. Within other embodiments, the amount of mRNA is detected using a hybridization technique, employing an oligonucleotide probe that hybridizes to a polynucleotide that encodes a polypeptide as recited above, or a complement of such a polynucleotide.

In related aspects, methods are provided for monitoring the progression of a cancer in a patient, comprising the steps of: (a) contacting a biological sample obtained from a patient with an oligonucleotide that hybridizes to a polynucleotide that encodes a polypeptide of the present invention; (b) detecting in the sample an amount of a polynucleotide that hybridizes to the oligonucleotide; (c) repeating steps (a) and (b) using a biological sample obtained from the patient at a subsequent point in time; and (d) comparing the amount of polynucleotide detected in step (c) with the amount detected in step (b) and therefrom monitoring the progression of the cancer in the patient.

Within further aspects, the present invention provides antibodies, such as monoclonal antibodies, that bind to a polypeptide as described above, as well as diagnostic kits comprising such antibodies. Diagnostic kits comprising one or more oligonucleotide probes or primers as described above are also provided.

These and other aspects of the present invention will become apparent upon reference to the following detailed description. All references disclosed herein are hereby incorporated by reference in their entirety as if each was incorporated individually.

### SEQUENCE IDENTIFIERS

SEQ ID NO: 1 is a first determined cDNA sequence for Contig 1, showing homology to Neutrophil Gelatinase Associated Lipocalin.

SEQ ID NO: 2 is the determined cDNA sequence for Contig 2, showing no significant homology to any known genes.

SEQ ID NO: 3 is the determined cDNA sequence for Contig 4, showing homology to Carcinoembryonic antigen.

SEQ ID NO: 4 is the determined cDNA sequence for Contig 5, showing homology to Carcinoembryonic antigen.

SEQ ID NO: 5 is the determined cDNA sequence for Contig 9, showing homology to Carcinoembryonic antigen.

SEQ ID NO: 6 is the determined cDNA sequence for Contig 52, showing homology to Carcinoembryonic antigen.

SEQ ID NO: 7 is the determined cDNA sequence for Contig 6, showing homology toVillin.

SEQ ID NO: 8 is the determined cDNA sequence for Contig 8, showing no significant homology to any known genes.

SEQ ID NO: 9 is the determined cDNA sequence for Contig 10, showing homology to Transforming Growth Factor (BIGH3).

SEQ ID NO: 10 is the determined cDNA sequence for Contig 19, showing homology to Transforming Growth Factor (BIGH3).

SEQ ID NO: 11 is the determined cDNA sequence for Contig 21, showing homology to Transforming Growth Factor (BIGH3).

SEQ ID NO: 12 is the determined cDNA sequence for Contig 11, showing homology to CO-029.

SEQ ID NO: 13 is the determined cDNA sequence for Contig 55, showing homology to CO-029.

SEQ ID NO: 14 is the determined cDNA sequence for Contig 12, showing homology to Chromosome 17, clone hRPC.1171_I_{—}10, also referred to as C798P.

SEQ ID NO: 15 is the determined cDNA sequence for Contig 13, showing no significant homology to any known gene.

SEQ ID NO: 16 is the determined cDNA sequence for Contig 14, also referred to as 14261, showing no significant homology to any known gene.

SEQ ID NO: 17 is the determined eDNA sequence for Contig 15, showing homology to Ets-Related Transcription Factor (ERT).

SEQ ID NO: 18 is the determined cDNA sequence for Contig 16, showing homology to Chromosome 5, PAC clone 228g9 (LBNL H142).

SEQ ID NO: 19 is the determined cDNA sequence for Contig 24, showing homology to Chromosome 5, PAC clone 228g9 (LBNL H142).

SEQ ID NO: 20 is the determined cDNA sequence for Contig 17, showing homology to Cytokeratin.

SEQ ID NO: 21 is the determined cDNA sequence for Contig 18, showing homology to L1-Cadherin.

SEQ ID NO: 22 is the determined cDNA sequence for Contig 20, showing no significant homology to any known gene.

SEQ ID NO: 23 is the determined cDNA sequence for Contig 22, showing homology to Bumetanide-sensitive Na—K—Cl cotransporter (NKCCl).

SEQ ID NO: 24 is the determined cDNA sequence for Contig 23, showing no significant homology to any known gene.

SEQ ID NO: 25 is the determined cDNA sequence for Contig 25, showing homology to Macrophage Inflammatory Protein 3 alpha.

SEQ ID NO: 26 is the determined cDNA sequence for Contig 26, showing homology to Laminin.

SEQ ID NO: 27 is the determined cDNA sequence for Contig 48, showing homology to Laminin.

SEQ ID NO: 28 is the determined cDNA sequence for Contig 27, showing homology to Mytobularin (MTM1).

SEQ ID NO: 29 is the determined cDNA sequence for Contig 28, showing homology to Chromosome 16 BAC clone CIT987SK-A-363E6.

SEQ ID NO: 30 is the determined cDNA sequence for Contig 29, also referred to as C751P and 14247, showing no significant homology to any known gene, but partial homology to Rat GSK-3-interacting protein Axil homolog.

SEQ ID NO: 31 is the determined cDNA sequence for Contig 30, showing homology to Zinc Finger Transcription Factor (ZNF207).

SEQ ID NO: 32 is the determined cDNA sequence for Contig 31, showing no significant homology to any known gene, but partial homology to Mus musculus GOB-4 homolog.

SEQ ID NO: 33 is the determined eDNA sequence for Contig 35, showing no significant homology to any known gene, but partial homology to Mus musculus GOB-4 homolog.

SEQ ID NO: 34 is the determined cDNA sequence for Contig 32, showing no significant homology to any known gene.

SEQ ID NO: 35 is the determined cDNA sequence for Contig 34, showing homology to Desmoglein 2.

SEQ ID NO: 36 is the determined cDNA sequence for Contig 36, showing no significant homology to any known gene.

SEQ ID NO: 37 is the determined cDNA sequence for Contig 37, showing homology to Putative Transmembrane Protein.

SEQ ID NO: 38 is the determined cDNA sequence for Contig 38, also referred to as C796P and 14219, showing no significant homology to any known gene.

SEQ ID NO: 39 is the determined cDNA sequence for Contig 40, showing homology to Nonspecific Cross-reacting Antigen.

SEQ ID NO: 40 is the determined cDNA sequence for Contig 41, also referred to as C799P and 14308, showing no significant homology to any known gene.

SEQ ID NO: 41 is the determined cDNA sequence for Contig 42, also referred to as C794P and 14309, showing no significant homology to any known gene.

SEQ ID NO: 42 is the determined eDNA sequence for Contig 43, showing homology to Chromosome 1 specific transcript KIAA0487.

SEQ ID NO: 43 is the determined cDNA sequence for Contig 45, showing homology to hMCM2.

SEQ ID NO: 44 is the determined cDNA sequence for Contig 46, showing homology to ETS2.

SEQ ID NO: 45 is the determined cDNA sequence for Contig 49, showing homology to Pump-1.

SEQ ID NO: 46 is the determined cDNA sequence for Contig 50, also referred to as C792P and 18323, showing no significant homology to any known gene.

SEQ ID NO: 47 is the determined cDNA sequence for Contig 51, also referred to as C795P and 14317, showing no significant homology to any known gene.

SEQ ID NO: 48 is the determined cDNA sequence for 11092, showing no significant homology to any known gene.

SEQ ID NO: 49 is the determined cDNA sequence for 11093, showing no significant homology to any known gene.

SEQ ID NO: 50 is the determined cDNA sequence for 11094, showing homology Human Putative Enterocyte Differentiation Protein.

SEQ ID NO: 51 is the determined cDNA sequence for 11095, showing homology to Human Transcriptional Corepressor hKAP1/TIF1B mRNA.

SEQ ID NO: 52 is the determined cDNA sequence for 11096, showing no significant homology to any known gene.

SEQ ID NO: 53 is the determined cDNA sequence for 11097, showing homology to Human Nonspecific Antigen.

SEQ ID NO: 54 is the determined cDNA sequence for 11098, showing no significant homology to any known gene.

SEQ ID NO: 55 is the determined cDNA sequence for 11099, showing homology to Human Pancreatic Secretory Inhibitor (PST) mRNA.

SEQ ID NO: 56 is the determined cDNA sequence for 11186, showing homology to Human Pancreatic Secretory Inhibitor (PST) mRNA.

SEQ ID NO: 57 is the determined cDNA sequence for 11101, showing homology to Human Chromosome X.

SEQ ID NO: 58 is the determined cDNA sequence for 11102, showing homology to Human Chromosome X.

SEQ ID NO: 59 is the determined cDNA sequence for 11103, showing no significant homology to any known gene.

SEQ ID NO: 60 is the determined cDNA sequence for 11174, showing no significant homology to any known gene.

SEQ ID NO: 61 is the determined cDNA sequence for 11104, showing homology to Human mRNA for KIAA0154.

SEQ ID NO: 62 is the determined cDNA sequence for 11105, showing homology toHuman Apurinic/Apyrimidinic Endonuclease (hap l)rnRNA.

SEQ ID NO: 63 is the determined cDNA sequence for 11106, showing homology toHuman Chromosome 12p13.

SEQ ID NO: 64 is the determined cDNA sequence for 11107, showing homology to Human 90 kDa Heat Shock Protein.

SEQ ID NO: 65 is the determined cDNA sequence for 11108, showing no significant homology to any known gene.

SEQ ID NO: 66 is the determined cDNA sequence for 11112, showing no significant homology to any known gene.

SEQ ID NO: 67 is the determined cDNA sequence for 11115, showing no significant homology to any known gene.

SEQ ID NO: 68 is the determined cDNA sequence for 11117, showing no significant homology to any known gene.

SEQ ID NO: 69 is the determined cDNA sequence for 11118, showing no significant homology to any known gene.

SEQ ID NO: 70 is the determined cDNA sequence for 11119, showing homology to Human Elongation Factor 1-alpha.

SEQ ID NO: 71 is the determined cDNA sequence for 11121, showing homology to Human Lamin B Receptor (LBR) mRNA.

SEQ ID NO: 72 is the determined cDNA sequence for 11122, showing homology to H. sapiens mRNA for Novel Glucocorticoid.

SEQ ID NO: 73 is the determined cDNA sequence for 11123, showing homology to H. sapiens mRNA for snRNP protein B.

SEQ ID NO: 74 is the determined cDNA sequence for 11124, showing homology to Human Cisplatin Resistance Associated Beta-protein.

SEQ ID NO: 75 is the determined cDNA sequence for 11127, showing homology to M. musculus Calumenin mRNA.

SEQ ID NO: 76 is the determined cDNA sequence for 11128, showing homology to Human ras-related small GTP binding protein.

SEQ ID NO: 77 is the determined cDNA sequence for 11130, showing homology to Human Cosmid U169d2.

SEQ ID NO: 78 is the determined cDNA sequence for 11131, showing homology to H. sapiens mRNA for protein homologous to Elongation 1-g.

SEQ ID NO: 79 is the determined cDNA sequence for 11134, showing no significant homology to any known gene.

SEQ ID NO: 80 is the determined EDNA sequence for 11135, showing homology to H. sapiens Nieman-Pick (NPC1) mRNA.

SEQ ID NO: 81 is the determined cDNA sequence for 11137, showing homology to H. sapiens mRNA for Niecin b-chain.

SEQ ID NO: 82 is the determined cDNA sequence for 11138, showing homology to Human Endogenous Retroviral Protease mRNA.

SEQ ID NO: 83 is the determined cDNA sequence for 11139, showing homology to H. sapiens mRNA for DMBT1 protein.

SEQ ID NO: 84 is the determined cDNA sequence for 11140, showing homology to H. sapiens ras GTPase activating-like protein.

SEQ ID NO: 85 is the determined cDNA sequence for 11143, showing homology to Human Acidic Ribosomal Phosphoprotein PO mRNA.

SEQ ID NO: 86 is the determined cDNA sequence for 11144, showing homology to H. sapiens U21 mRNA.

SEQ ID NO: 87 is the determined cDNA sequence for 11145, showing homology to Human GTP-binding protein.

SEQ ID NO: 88 is the determined cDNA sequence for 11148, showing homology to H. sapiens U21 mRNA.

SEQ ID NO: 89 is the determined cDNA sequence for 11151, showing no significant homology to any known gene.

SEQ ID NO: 90 is the determined cDNA sequence for 11154, showing no significant homology to any known gene.

SEQ ID NO: 91 is the determined cDNA sequence for 11156, showing homology to H. sapiens Ribosomal Protein L27.

SEQ ID NO: 92 is the determined cDNA sequence for 11157, showing homology to H. sapiens Ribosomal Protein L27.

SEQ ID NO: 93 is the determined cDNA sequence for 11158, showing no significant homology to any known gene.

SEQ ID NO: 94 is the determined cDNA sequence for 11162, showing homology to Ag-X antigen.

SEQ ID NO: 95 is the determined cDNA sequence for 11164, showing homology to H. sapiens mRNA for Signal Recognition Protein sub 14.

SEQ ID NO: 96 is the determined cDNA sequence for 11165, showing homology to Human PAC 204e5/127h14.

SEQ ID NO: 97 is the determined cDNA sequence for 11166, showing homology to Human mRNA for KIAA0108.

SEQ ID NO: 98 is the determined cDNA sequence for 11167, showing homology to H. sapiens mRNA for Neutrophil Gelatinase asset. Lipocalin.

SEQ ID NO: 99 is the determined cDNA sequence for 11168, showing no significant homology to any known gene.

SEQ ID NO: 100 is the determined cDNA sequence for 11172, showing no significant homology to any known gene.

SEQ ID NO: 101 is the determined cDNA sequence for 11175, showing no significant homology to any known gene.

SEQ ID NO: 102 is the determined cDNA sequence for 11176, showing homology to Human maspin mRNA.

SEQ ID NO: 103 is the determined cDNA sequence for 11177, showing homology to Human Carcinoembryonic Antigen.

SEQ ID NO: 104 is the determined cDNA sequence for 11178, showing homology to Human A-Tubulin mRNA.

SEQ ID NO: 105 is the determined cDNA sequence for 11179, showing homology to Human mRNA for proton-ATPase-like protein.

SEQ ID NO: 106 is the determined cDNA sequence for 11180, showing homology to Human HepG2 3′ region cDNA clone hmd.

SEQ ID NO: 107 is the determined cDNA sequence for 11182, showing homology to Human MHC homologous to Chicken B-Complex Protein.

SEQ ID NO: 108 is the determined cDNA sequence for 11183, showing homology to Human High Mobility Group Box (SSRP1) mRNA.

SEQ ID NO: 109 is the determined cDNA sequence for 11184, showing no significant homology to any known gene.

SEQ ID NO: 110 is the determined cDNA sequence for 11185, showing no significant homology to any known gene.

SEQ ID NO: 111 is the determined cDNA sequence for 11187, showing no significant homology to any known gene.

SEQ ID NO: 112 is the determined cDNA sequence for 11190, showing homology to Human Replication Protein A 70 kDa.

SEQ ID NO: 113 is the determined cDNA sequence for Contig 47, also referred to as C797P, showing homology to Human Chromosome X clone bWXD342.

SEQ ID NO: 114 is the determined cDNA sequence for Contig 7, showing homology to Equilibrative Nucleoside Transporter 2 (ent2).

SEQ ID NO: 115 is the determined cDNA sequence for 14235.1, also referred to as C791P, showing homology to H. sapiens chromosome 21 derived BAC containing ets-2 gene.

SEQ ID NO: 116 is the determined cDNA sequence for 14287.2, showing no significant homology to any known gene, but some degree of homology to Putative Transmembrane Protein.

SEQ ID NO: 117 is the determined cDNA sequence for 14233.1, also referred to as Contig 48, showing no significant homology to any known gene.

SEQ ID NO: 118 is the determined cDNA sequence for 14298.2, also referred to as C793P, showing no significant homology to any known gene.

SEQ ID NO: 119 is the determined cDNA sequence for 14372, also referred to as Contig 44, showing no significant homology to any known gene.

SEQ ID NO: 120 is the determined cDNA sequence for 14295, showing homology to secreted cement gland protein XAG-2 homolog.

SEQ ID NO: 121 is the determined full-length cDNA sequence for a clone showing homology to Beta IG-H3.

SEQ ID NO: 122 is the predicted amino acid sequence for the clone of SEQ ID NO: 121.

SEQ ID NO: 123 is a longer determined cDNA sequence for C751P.

SEQ ID NO: 124 is a longer determined cDNA sequence for C791P.

SEQ ID NO: 125 is a longer determined cDNA sequence for C792P.

SEQ ID NO: 126 is a longer determined cDNA sequence for C793P.

SEQ ID NO: 127 is a longer determined cDNA sequence for C794P.

SEQ ID NO: 128 is a longer determined cDNA sequence for C795P.

SEQ ID NO: 129 is a longer determined cDNA sequence for C796P.

SEQ ID NO: 130 is a longer determined cDNA sequence for C797P.

SEQ ID NO: 131 is a longer determined cDNA sequence for C798P.

SEQ ID NO: 132 is a longer determined cDNA sequence for C799P.

SEQ ID NO: 133 is a first partial determined cDNA sequence for CoSub-3 (also known as 23569).

SEQ ID NO: 134 is a second partial determined cDNA sequence for CoSub-3 (also known as 23569).

SEQ ID NO: 135 is a first partial determined cDNA sequence for CoSub-13 (also known as 23579).

SEQ ID NO: 136 is a second partial determined cDNA sequence for CoSub-13 (also known as 23579).

SEQ ID NO: 137 is the determined cDNA sequence for CoSub-17 (also known as 23583).

SEQ ID NO: 138 is the determined cDNA sequence for CoSub-19 (also known as 23585).

SEQ ID NO: 139 is the determined cDNA sequence for CoSub-22 (also known as 23714).

SEQ ID NO: 140 is the determined cDNA sequence for CoSub-23 (also known as 23715).

SEQ ID NO: 141 is the determined cDNA sequence for CoSub-26 (also known as 23717).

SEQ ID NO: 142 is the determined cDNA sequence for CoSub-33 (also known as 23724).

SEQ ID NO: 143 is the determined cDNA sequence for CoSub-34 (also known as 23725).

SEQ ID NO: 144 is the determined cDNA sequence for CoSub-35 (also known as 23726).

SEQ ID NO: 145 is the determined cDNA sequence for CoSub-37 (also known as 23728).

SEQ ID NO: 146 is the determined cDNA sequence for CoSub-39 (also known as 23730).

SEQ ID NO: 147 is the determined cDNA sequence for CoSub-42 (also known as 23766).

SEQ ID NO: 148 is the determined cDNA sequence for CoSub-44 (also known as 23768).

SEQ ID NO: 149 is the determined cDNA sequence for CoSub-47 (also known as 23771).

SEQ ID NO: 150 is the determined cDNA sequence for CoSub-54 (also known as 23778).

SEQ ID NO: 151 is the determined cDNA sequence for CoSub-55 (also known as 23779).

SEQ ID NO: 152 is the determined cDNA sequence for CT1 (also known as 24099).

SEQ ID NO: 153 is the determined cDNA sequence for CT2 (also known as 24100).

SEQ ID NO: 154 is the determined cDNA sequence for CT3 (also known as 24101).

SEQ ID NO: 155 is the determined cDNA sequence for CT6 (also known as 24104).

SEQ ID NO: 156 is the determined cDNA sequence for CT7 (also known as 24105).

SEQ ID NO: 157 is the determined cDNA sequence for CT12 (also known as 24110).

SEQ ID NO: 158 is the determined cDNA sequence for CT13 (also known as 20 24111).

SEQ ID NO: 159 is the determined cDNA sequence for CT14 (also known as 24112).

SEQ ID NO: 160 is the determined cDNA sequence for CT15 (also known as 24113).

SEQ ID NO: 161 is the determined cDNA sequence for CT17 (also known as 24115).

SEQ ID NO: 162 is the determined cDNA sequence for CT18 (also known as 24116).

SEQ ID NO: 163 is the determined cDNA sequence for CT22 (also known as 30 23848).

SEQ ID NO: 164 is the determined cDNA sequence for CT24 (also known as 23849).

SEQ ID NO: 165 is the determined cDNA sequence for CT31 (also known as 23854).

SEQ ID NO: 166 is the determined cDNA sequence for CT34 (also known as 23856).

SEQ ID NO: 167 is the determined cDNA sequence for CT37 (also known as 23859).

SEQ ID NO: 168 is the determined cDNA sequence for CT39 (also known as 23860).

SEQ ID NO: 169 is the determined cDNA sequence for CT40 (also known as 23861).

SEQ ID NO: 170 is the determined cDNA sequence for CT51 (also known as 24130).

SEQ ID NO: 171 is the determined cDNA sequence for CT53 (also known as 24132).

SEQ ID NO: 172 is the determined cDNA sequence for CT63 (also known as 24595).

SEQ ID NO: 173 is the determined cDNA sequence for CT88 (also known as 24608).

SEQ ID NO: 174 is the determined cDNA sequence for CT92 (also known as 24800).

SEQ ID NO: 175 is the determined cDNA sequence for CT94 (also known as 24802).

SEQ ID NO: 176 is the determined cDNA sequence for CT102 (also known as 24805).

SEQ ID NO: 177 is the determined cDNA sequence for CT103 (also known as 24806).

SEQ ID NO: 178 is the determined cDNA sequence for CT111 (also known as 25520).

SEQ ID NO: 179 is the determined cDNA sequence for CT118 (also known as 25522).

SEQ ID NO: 180 is the determined cDNA sequence for CT121 (also known as 25523).

SEQ ID NO: 181 is the determined cDNA sequence for CT126 (also known as 25527).

SEQ ID NO: 182 is the determined cDNA sequence for CT135 (also known as 25534).

SEQ ID NO: 183 is the determined cDNA sequence for CT140 (also known as 25537).

SEQ ID NO: 184 is the determined cDNA sequence for CT145 (also known as 25542).

SEQ ID NO: 185 is the determined cDNA sequence for CT147 (also known as 25543).

SEQ ID NO: 186 is the determined cDNA sequence for CT148 (also known as 25544).

SEQ ID NO: 187 is the determined cDNA sequence for CT502 (also known as 26420).

SEQ ID NO: 188 is the determined cDNA sequence for CT507 (also known as 26425).

SEQ ID NO: 189 is the determined cDNA sequence for CT521 (also known as 27366).

SEQ ID NO: 190 is the determined cDNA sequence for CT544 (also known as 27375).

SEQ ID NO: 191 is the determined cDNA sequence for CT577 (also known as 27385).

SEQ ID NO: 192 is the determined cDNA sequence for CT580 (also known as 27387).

SEQ ID NO: 193 is the determined cDNA sequence for CT594 (also known as 27540).

SEQ ID NO: 194 is the determined cDNA sequence for CT606 (also known as 27547).

SEQ ID NO: 195 is the determined cDNA sequence for CT607 (also known as 27548).

SEQ ID NO: 196 is the determined cDNA sequence for CT599 (also known as 27903).

SEQ ID NO: 197 is the determined cDNA sequence for CT632 (also known as 27922).

SEQ ID NO: 198 is the predicted amino acid sequence for CT502 (SEQ ID NO: 187).

SEQ ID NO: 199 is the predicted amino acid sequence for CT507 (SEQ ID NO: 188).

SEQ ID NO: 200 is the predicted amino acid sequence for CT521 (SEQ ID NO: 189).

SEQ ID NO: 201 is the predicted amino acid sequence for CT544 (SEQ ID NO: 190).

SEQ ID NO: 202 is the predicted amino acid sequence for CT606 (SEQ ID NO: 194).

SEQ ID NO: 203 is the predicted amino acid sequence for CT607 (SEQ ID NO: 195).

SEQ ID NO: 204 is the predicted amino acid sequence for CT632 (SEQ ID NO: 197).

SEQ ID NO: 205 is the determined cDNA sequence for clone 25244.

SEQ ID NO: 206 is the determined cDNA sequence for clone 25245.

SEQ ID NO: 207 is the determined cDNA sequence for clone 25246.

SEQ ID NO: 208 is the determined cDNA sequence for clone 25248.

SEQ ID NO: 209 is the determined cDNA sequence for clone 25249.

SEQ ID NO: 210 is the determined cDNA sequence for clone 25250.

SEQ ID NO: 211 is the determined cDNA sequence for clone 25251.

SEQ ID NO: 212 is the determined cDNA sequence for clone 25252.

SEQ ID NO: 213 is the determined cDNA sequence for clone 25253.

SEQ ID NO: 214 is the determined cDNA sequence for clone 25254.

SEQ ID NO: 215 is the determined cDNA sequence for clone 25255.

SEQ ID NO: 216 is the determined cDNA sequence for clone 25256.

SEQ ID NO: 217 is the determined cDNA sequence for clone 25257.

SEQ ID NO: 218 is the determined cDNA sequence for clone 25259.

SEQ ID NO: 219 is the determined cDNA sequence for clone 25260.

SEQ ID NO: 220 is the determined cDNA sequence for clone 25261.

SEQ ID NO: 221 is the determined cDNA sequence for clone 25262.

SEQ ID NO: 222 is the determined cDNA sequence for clone 25263.

SEQ ID NO: 223 is the determined cDNA sequence for clone 25264.

SEQ ID NO: 224 is the determined cDNA sequence for clone 25265.

SEQ ID NO: 225 is the determined cDNA sequence for clone 25266.

SEQ ID NO: 226 is the determined cDNA sequence for clone 25267.

SEQ ID NO: 227 is the determined cDNA sequence for clone 25268.

SEQ ID NO: 228 is the determined cDNA sequence for clone 25269.

SEQ ID NO: 229 is the determined cDNA sequence for clone 25271.

SEQ ID NO: 230 is the determined cDNA sequence for clone 25272.

SEQ ID NO: 231 is the determined cDNA sequence for clone 25273.

SEQ ID NO: 232 is the determined cDNA sequence for clone 25274.

SEQ ID NO: 233 is the determined cDNA sequence for clone 25275.

SEQ ID NO: 234 is the determined cDNA sequence for clone 25276.

SEQ ID NO: 235 is the determined cDNA sequence for clone 25277.

SEQ ID NO: 236 is the determined cDNA sequence for clone 25278.

SEQ ID NO: 237 is the determined cDNA sequence for clone 25280.

SEQ ID NO: 238 is the determined cDNA sequence for clone 25281.

SEQ ID NO: 239 is the determined cDNA sequence for clone 25282.

SEQ ID NO: 240 is the determined cDNA sequence for clone 25283.

SEQ ID NO: 241 is the determined cDNA sequence for clone 25284.

SEQ ID NO: 242 is the determined cDNA sequence for clone 25285.

SEQ ID NO: 243 is the determined cDNA sequence for clone 25286.

SEQ ID NO: 244 is the determined cDNA sequence for clone 25287.

SEQ ID NO: 245 is the determined cDNA sequence for clone 25288.

SEQ ID NO: 246 is the determined cDNA sequence for clone 25289.

SEQ ID NO: 247 is the determined cDNA sequence for clone 25290.

SEQ ID NO: 248 is the determined cDNA sequence for clone 25291.

SEQ ID NO: 249 is the determined cDNA sequence for clone 25292.

SEQ ID NO: 250 is the determined cDNA sequence for clone 25293.

SEQ ID NO: 251 is the determined cDNA sequence for clone 25294.

SEQ ID NO: 252 is the determined cDNA sequence for clone 25295.

SEQ ID NO: 253 is the determined cDNA sequence for clone 25296.

SEQ ID NO: 254 is the determined cDNA sequence for clone 25297.

SEQ ID NO: 255 is the determined cDNA sequence for clone 25418.

SEQ ID NO: 256 is the determined cDNA sequence for clone 25419.

SEQ ID NO: 257 is the determined cDNA sequence for clone 25420.

SEQ ID NO: 258 is the determined cDNA sequence for clone 25421.

SEQ ID NO: 259 is the determined cDNA sequence for clone 25422.

SEQ ID NO: 260 is the determined cDNA sequence for clone 25423.

SEQ ID NO: 261 is the determined cDNA sequence for clone 25424.

SEQ ID NO: 262 is the determined cDNA sequence for clone 25426.

SEQ ID NO: 263 is the determined cDNA sequence for clone 25427.

SEQ ID NO: 264 is the determined cDNA sequence for clone 25428.

SEQ ID NO: 265 is the determined cDNA sequence for clone 25429.

SEQ ID NO: 266 is the determined cDNA sequence for clone 25430.

SEQ ID NO: 267 is the determined cDNA sequence for clone 25431.

SEQ ID NO: 268 is the determined cDNA sequence for clone 25432.

SEQ ID NO: 269 is the determined cDNA sequence for clone 25433.

SEQ ID NO: 270 is the determined cDNA sequence for clone 25434.

SEQ ID NO: 271 is the determined cDNA sequence for clone 25435.

SEQ ID NO: 272 is the determined cDNA sequence for clone 25436.

SEQ ID NO: 273 is the determined cDNA sequence for clone 25437.

SEQ ID NO: 274 is the determined cDNA sequence for clone 25438.

SEQ ID NO: 275 is the determined cDNA sequence for clone 25439.

SEQ ID NO: 276 is the determined cDNA sequence for clone 25440.

SEQ ID NO: 277 is the determined cDNA sequence for clone 25441.

SEQ ID NO: 278 is the determined cDNA sequence for clone 25442.

SEQ ID NO: 279 is the determined cDNA sequence for clone 25443.

SEQ ID NO: 280 is the determined cDNA sequence for clone 25444.

SEQ ID NO: 281 is the determined cDNA sequence for clone 25445.

SEQ ID NO: 282 is the determined cDNA sequence for clone 25446.

SEQ ID NO: 283 is the determined cDNA sequence for clone 25447.

SEQ ID NO: 284 is the determined cDNA sequence for clone 25448.

SEQ ID NO: 285 is the determined cDNA sequence for clone 25844.

SEQ ID NO: 286 is the determined cDNA sequence for clone 25845.

SEQ ID NO: 287 is the determined cDNA sequence for clone 25846.

SEQ ID NO: 288 is the determined cDNA sequence for clone 25847.

SEQ ID NO: 289 is the determined cDNA sequence for clone 25848.

SEQ ID NO: 290 is the determined cDNA sequence for clone 25850.

SEQ ID NO: 291 is the determined cDNA sequence for clone 25851.

SEQ ID NO: 292 is the determined cDNA sequence for clone 25852.

SEQ ID NO: 293 is the determined cDNA sequence for clone 25853.

SEQ ID NO: 294 is the determined cDNA sequence for clone 25854.

SEQ ID NO: 295 is the determined cDNA sequence for clone 25855.

SEQ ID NO: 296 is the determined cDNA sequence for clone 25856.

SEQ ID NO: 297 is the determined cDNA sequence for clone 25857.

SEQ ID NO: 298 is the determined cDNA sequence for clone 25858.

SEQ ID NO: 299 is the determined cDNA sequence for clone 25859.

SEQ ID NO: 300 is the determined cDNA sequence for clone 25860.

SEQ ID NO: 301 is the determined cDNA sequence for clone 25861.

SEQ ID NO: 302 is the determined cDNA sequence for clone 25862.

SEQ ID NO: 303 is the determined cDNA sequence for clone 25863.

SEQ ID NO: 304 is the determined cDNA sequence for clone 25864.

SEQ ID NO: 305 is the determined cDNA sequence for clone 25865.

SEQ ID NO: 306 is the determined cDNA sequence for clone 25866.

SEQ ID NO: 307 is the determined cDNA sequence for clone 25867.

SEQ ID NO: 308 is the determined cDNA sequence for clone 25868.

SEQ ID NO: 309 is the determined cDNA sequence for clone 25869.

SEQ ID NO: 310 is the determined cDNA sequence for clone 25870.

SEQ ID NO: 311 is the determined cDNA sequence for clone 25871.

SEQ ID NO: 312 is the determined cDNA sequence for clone 25872.

SEQ ID NO: 313 is the determined cDNA sequence for clone 25873.

SEQ ID NO: 314 is the determined cDNA sequence for clone 25875.

SEQ ID NO: 315 is the determined cDNA sequence for clone 25876.

SEQ ID NO: 316 is the determined cDNA sequence for clone 25877.

SEQ ID NO: 317 is the determined cDNA sequence for clone 25878.

SEQ ID NO: 318 is the determined cDNA sequence for clone 25879.

SEQ ID NO: 319 is the determined cDNA sequence for clone 25880.

SEQ ID NO: 320 is the determined cDNA sequence for clone 25881.

SEQ ID NO: 321 is the determined cDNA sequence for clone 25882.

SEQ ID NO: 322 is the determined cDNA sequence for clone 25883.

SEQ ID NO: 323 is the determined cDNA sequence for clone 25884.

SEQ ID NO: 324 is the determined cDNA sequence for clone 25885.

SEQ ID NO: 325 is the determined cDNA sequence for clone 25886.

SEQ ID NO: 326 is the determined cDNA sequence for clone 25887.

SEQ ID NO: 327 is the determined cDNA sequence for clone 25888.

SEQ ID NO: 328 is the determined cDNA sequence for clone 25889.

SEQ ID NO: 329 is the determined cDNA sequence for clone 25890.

SEQ ID NO: 330 is the determined cDNA sequence for clone 25892.

SEQ ID NO: 331 is the determined cDNA sequence for clone 25894.

SEQ ID NO: 332 is the determined cDNA sequence for clone 25895.

SEQ ID NO: 333 is the determined cDNA sequence for clone 25896.

SEQ ID NO: 334 is the determined cDNA sequence for clone 25897.

SEQ ID NO: 335 is the determined cDNA sequence for clone 25899.

SEQ ID NO: 336 is the determined cDNA sequence for clone 25900.

SEQ ID NO: 337 is the determined cDNA sequence for clone 25901.

SEQ ID NO: 338 is the determined cDNA sequence for clone 25902.

SEQ ID NO: 339 is the determined cDNA sequence for clone 25903.

SEQ ID NO: 340 is the determined cDNA sequence for clone 25904.

SEQ ID NO: 341 is the determined cDNA sequence for clone 25906.

SEQ ID NO: 342 is the determined cDNA sequence for clone 25907.

SEQ ID NO: 343 is the determined cDNA sequence for clone 25908.

SEQ ID NO: 344 is the determined cDNA sequence for clone 25909.

SEQ ID NO: 345 is the determined cDNA sequence for clone 25910.

SEQ ID NO: 346 is the determined cDNA sequence for clone 25911.

SEQ ID NO: 347 is the determined cDNA sequence for clone 25912.

SEQ ID NO: 348 is the determined cDNA sequence for clone 25913.

SEQ ID NO: 349 is the determined cDNA sequence for clone 25914.

SEQ ID NO: 350 is the determined cDNA sequence for clone 25915.

SEQ ID NO: 351 is the determined cDNA sequence for clone 25916.

SEQ ID NO: 352 is the determined cDNA sequence for clone 25917.

SEQ ID NO: 353 is the determined cDNA sequence for clone 25918.

SEQ ID NO: 354 is the determined cDNA sequence for clone 25919.

SEQ ID NO: 355 is the determined cDNA sequence for clone 25920.

SEQ ID NO: 356 is the determined cDNA sequence for clone 25921.

SEQ ID NO: 357 is the determined cDNA sequence for clone 25922.

SEQ ID NO: 358 is the determined cDNA sequence for clone 25924.

SEQ ID NO: 359 is the determined cDNA sequence for clone 25925.

SEQ ID NO: 360 is the determined cDNA sequence for clone 25926.

SEQ ID NO: 361 is the determined cDNA sequence for clone 25927.

SEQ ID NO: 362 is the determined cDNA sequence for clone 25928.

SEQ ID NO: 363 is the determined cDNA sequence for clone 25929.

SEQ ID NO: 364 is the determined cDNA sequence for clone 25930.

SEQ ID NO: 365 is the determined cDNA sequence for clone 25931.

SEQ ID NO: 366 is the determined cDNA sequence for clone 25932.

SEQ ID NO: 367 is the determined cDNA sequence for clone 25933.

SEQ ID NO: 368 is the determined cDNA sequence for clone 25934.

SEQ ID NO: 369 is the determined cDNA sequence for clone 25935.

SEQ ID NO: 370 is the determined cDNA sequence for clone 25936.

SEQ ID NO: 371 is the determined cDNA sequence for clone 25939.

SEQ ID NO: 372 is the determined cDNA sequence for clone 32016.

SEQ ID NO: 373 is the determined cDNA sequence for clone 32021.

SEQ ID NO: 374 is the determined cDNA sequence for clone 31993.

SEQ ID NO: 375 is the determined cDNA sequence for clone 31997.

SEQ ID NO: 376 is the determined cDNA sequence for clone 31942.

SEQ ID NO: 377 is the determined cDNA sequence for clone 31937.

SEQ ID NO: 378 is the determined cDNA sequence for clone 31952.

SEQ ID NO: 379 is the determined cDNA sequence for clone 31992.

SEQ ID NO: 380 is the determined cDNA sequence for clone 31961.

SEQ ID NO: 381 is the determined cDNA sequence for clone 31964.

SEQ ID NO: 382 is the determined cDNA sequence for clone 32005.

SEQ ID NO: 383 is the determined cDNA sequence for clone 31980.

SEQ ID NO: 384 is the determined cDNA sequence for clone 31940.

SEQ ID NO: 385 is the determined cDNA sequence for clone 32004.

SEQ ID NO: 386 is the determined cDNA sequence for clone 31956.

SEQ ID NO: 387 is the determined cDNA sequence for clone 31934.

SEQ ID NO: 388 is the determined cDNA sequence for clone 31998.

SEQ ID NO: 389 is the determined cDNA sequence for clone 31973.

SEQ ID NO: 390 is the determined cDNA sequence for clone 31976.

SEQ ID NO: 391 is the determined cDNA sequence for clone 31988.

SEQ ID NO: 392 is the determined cDNA sequence for clone 31948.

SEQ ID NO: 393 is the determined cDNA sequence for clone 32013.

SEQ ID NO: 394 is the determined cDNA sequence for clone 31986.

SEQ ID NO: 395 is the determined cDNA sequence for clone 31954.

SEQ ID NO: 396 is the determined cDNA sequence for clone 31987.

SEQ ID NO: 397 is the determined cDNA sequence for clone 32029.

SEQ ID NO: 398 is the determined cDNA sequence for clone 32028.

SEQ ID NO: 399 is the determined cDNA sequence for clone 32012.

SEQ ID NO: 400 is the determined cDNA sequence for clone 31959.

SEQ ID NO: 401 is the determined cDNA sequence for clone 32027.

SEQ ID NO: 402 is the determined cDNA sequence for clone 31957.

SEQ ID NO: 403 is the determined cDNA sequence for clone 31950.

SEQ ID NO: 404 is the determined cDNA sequence for clone 32011.

SEQ ID NO: 405 is the determined cDNA sequence for clone 32022.

SEQ ID NO: 406 is the determined cDNA sequence for clone 32014.

SEQ ID NO: 407 is the determined cDNA sequence for clone 31963.

SEQ ID NO: 408 is the determined cDNA sequence for clone 31989.

SEQ ID NO: 409 is the determined cDNA sequence for clone 32015.

SEQ ID NO: 410 is the determined cDNA sequence for clone 32002.

SEQ ID NO: 411 is the determined cDNA sequence for clone 31939.

SEQ ID NO: 412 is the determined cDNA sequence for clone 32003.

SEQ ID NO: 413 is the determined cDNA sequence for clone 31936.

SEQ ID NO: 414 is the determined cDNA sequence for clone 32007.

SEQ ID NO: 415 is the determined cDNA sequence for clone 31965.

SEQ ID NO: 416 is the determined cDNA sequence for clone 31935.

SEQ ID NO: 417 is the determined cDNA sequence for clone 32008.

SEQ ID NO: 418 is the determined cDNA sequence for clone 31966.

SEQ ID NO: 419 is the determined cDNA sequence for clone 32020.

SEQ ID NO: 420 is the determined cDNA sequence for clone 31971.

SEQ ID NO: 421 is the determined cDNA sequence for clone 31977.

SEQ ID NO: 422 is the determined cDNA sequence for clone 31985.

SEQ ID NO: 423 is the determined cDNA sequence for clone 32023.

SEQ ID NO: 424 is the determined cDNA sequence for clone 31981.

SEQ ID NO: 425 is the determined cDNA sequence for clone 32006.

SEQ ID NO: 426 is the determined cDNA sequence for clone 31991.

SEQ ID NO: 427 is the determined cDNA sequence for clone 31995.

SEQ ID NO: 428 is the determined cDNA sequence for clone 32000.

SEQ ID NO: 429 is the determined cDNA sequence for clone 31990.

SEQ ID NO: 430 is the determined cDNA sequence for clone 31946.

SEQ ID NO: 431 is the determined cDNA sequence for clone 31938.

SEQ ID NO: 432 is the determined cDNA sequence for clone 31941.

SEQ ID NO: 433 is the determined cDNA sequence for clone 31982.

SEQ ID NO: 434 is the determined cDNA sequence for clone 31996.

SEQ ID NO: 435 is the determined cDNA sequence for clone 32010.

SEQ ID NO: 436 is the determined cDNA sequence for clone 31974.

SEQ ID NO: 437 is the determined cDNA sequence for clone 31983.

SEQ ID NO: 438 is the determined cDNA sequence for clone 31999.

SEQ ID NO: 439 is the determined cDNA sequence for clone 31949.

SEQ ID NO: 440 is the determined cDNA sequence for clone 31947.

SEQ ID NO: 441 is the determined cDNA sequence for clone 31994.

SEQ ID NO: 442 is the determined cDNA sequence for clone 31958.

SEQ ID NO: 443 is the determined cDNA sequence for clone 31975.

SEQ ID NO: 444 is the determined cDNA sequence for clone 31984.

SEQ ID NO: 445 is the determined cDNA sequence for clone 32024.

SEQ ID NO: 446 is the determined cDNA sequence for clone 31972.

SEQ ID NO: 447 is the determined cDNA sequence for clone 31943.

SEQ ID NO: 448 is the determined cDNA sequence for clone 32018.

SEQ ID NO: 449 is the determined cDNA sequence for clone 32026.

SEQ ID NO: 450 is the determined cDNA sequence for clone 32009.

SEQ ID NO: 451 is the determined cDNA sequence for clone 32019.

SEQ ID NO: 452 is the determined cDNA sequence for clone 32025.

SEQ ID NO: 453 is the determined cDNA sequence for clone 31967.

SEQ ID NO: 454 is the determined cDNA sequence for clone 31968.

SEQ ID NO: 455 is the determined cDNA sequence for clone 31955.

SEQ ID NO: 456 is the determined cDNA sequence for clone 31951.

SEQ ID NO: 457 is the determined cDNA sequence for clone 31970.

SEQ ID NO: 458 is the determined cDNA sequence for clone 31962.

SEQ ID NO: 459 is the determined cDNA sequence for clone 32001.

SEQ ID NO: 460 is the determined cDNA sequence for clone 31953.

SEQ ID NO: 461 is the determined cDNA sequence for clone 31944.

SEQ ID NO: 462 is the determined cDNA sequence for clone 31825.

SEQ ID NO: 463 is the determined cDNA sequence for clone 31828.

SEQ ID NO: 464 is the determined cDNA sequence for clone 31830.

SEQ ID NO: 465 is the determined cDNA sequence for clone 31841.

SEQ ID NO: 466 is the determined cDNA sequence for clone 31847.

SEQ ID NO: 467 is the determined cDNA sequence for clone 31850.

SEQ ID NO: 468 is the determined cDNA sequence for clone 31852.

SEQ ID NO: 469 is the determined cDNA sequence for clone 31855.

SEQ ID NO: 470 is the determined cDNA sequence for clone 31858.

SEQ ID NO: 471 is the determined cDNA sequence for clone 31861.

SEQ ID NO: 472 is the determined cDNA sequence for clone 31868.

SEQ ID NO: 473 is the determined cDNA sequence for clone 31870.

SEQ ID NO: 474 is the determined cDNA sequence for clone 31872.

SEQ ID NO: 475 is the determined cDNA sequence for clone 31873.

SEQ ID NO: 476 is the determined cDNA sequence for clone 31877.

SEQ ID NO: 477 is the determined cDNA sequence for clone 31878.

SEQ ID NO. 478 is the determined cDNA sequence for clone 31885.

SEQ ID NO: 479 is the determined cDNA sequence for clone 31888.

SEQ ID NO: 480 is the determined cDNA sequence for clone 31890.

SEQ ID NO: 481 is the determined cDNA sequence for clone 31893.

SEQ ID NO: 482 is the determined cDNA sequence for clone 31898.

SEQ ID NO: 483 is the determined cDNA sequence for clone 31901.

SEQ ID NO: 484 is the determined cDNA sequence for clone 31909.

SEQ ID NO: 485 is the determined cDNA sequence for clone 31910.

SEQ ID NO: 486 is the determined cDNA sequence for clone 31914.

SEQ ID NO: 487 is the determined cDNA sequence for contig 1.

SEQ ID NO: 488 is the determined cDNA sequence for contig 2.

SEQ ID NO: 489 is the determined cDNA sequence for contig 3.

SEQ ID NO: 490 is the determined cDNA sequence for contig 4.

SEQ ID NO: 491 is the determined cDNA sequence for contig 5.

SEQ ID NO: 492 is the determined cDNA sequence for contig 6.

SEQ ID NO: 493 is the determined cDNA sequence for contig 7.

SEQ ID NO: 494 is the determined cDNA sequence for contig 8.

SEQ ID NO: 495 is the determined cDNA sequence for contig 9.

SEQ ID NO: 496 is the determined cDNA sequence for contig 10.

SEQ ID NO: 497 is the determined cDNA sequence for contig 11.

SEQ ID NO: 498 is the determined cDNA sequence for contig 12.

SEQ ID NO: 499 is the determined cDNA sequence for contig 13.

SEQ ID NO: 500 is the determined cDNA sequence for contig 14.

SEQ ID NO: 501 is the determined cDNA sequence for contig 15.

SEQ ID NO: 502 is the determined cDNA sequence for contig 16.

SEQ ID NO: 503 is the determined cDNA sequence for contig 17.

SEQ ID NO: 504 is the determined cDNA sequence for contig 18.

SEQ ID NO: 505 is the determined cDNA sequence for contig 19.

SEQ ID NO: 506 is the determined cDNA sequence for contig 20.

SEQ ID NO: 507 is the determined cDNA sequence for contig 21.

SEQ ID NO: 508 is the determined cDNA sequence for contig 22.

SEQ ID NO: 509 is the determined cDNA sequence for contig 23.

SEQ ID NO: 510 is the determined cDNA sequence for contig 24.

SEQ ID NO: 511 is the determined cDNA sequence for contig 25.

SEQ ID NO: 512 is the determined cDNA sequence for contig 26.

SEQ ID NO: 513 is the determined cDNA sequence for contig 27.

SEQ ID NO: 514 is the determined cDNA sequence for contig 28.

SEQ ID NO: 515 is the determined cDNA sequence for contig 29.

SEQ ID NO: 516 is the determined cDNA sequence for contig 30.

SEQ ID NO: 517 is the determined cDNA sequence for contig 31.

SEQ ID NO: 518 is the determined cDNA sequence for contig 32.

SEQ ID NO: 519 is the determined cDNA sequence for contig 33.

SEQ ID NO: 520 is the determined cDNA sequence for contig 34.

SEQ ID NO: 521 is the determined cDNA sequence for contig 35.

SEQ ID NO: 522 is the determined cDNA sequence for contig 36.

SEQ ID NO: 523 is the determined cDNA sequence for contig 37.

SEQ ID NO: 524 is the determined cDNA sequence for contig 38.

SEQ ID NO: 525 is the determined cDNA sequence for contig 39.

SEQ ID NO: 526 is the determined cDNA sequence for contig 40.

SEQ ID NO: 527 is the determined cDNA sequence for contig 41.

SEQ ID NO: 528 is the determined cDNA sequence for contig 42.

SEQ ID NO: 529 is the determined cDNA sequence for contig 43.

SEQ ID NO: 530 is the determined cDNA sequence for contig 44.

SEQ ID NO: 531 is the determined cDNA sequence for contig 45.

SEQ ID NO: 532 is the determined cDNA sequence for contig 46.

SEQ ID NO: 533 is the determined cDNA sequence for contig 47.

SEQ ID NO: 534 is the determined cDNA sequence for contig 48.

SEQ ID NO: 535 is the determined cDNA sequence for contig 49.

SEQ ID NO: 536 is the determined cDNA sequence for contig 50.

SEQ ID NO: 537 is the determined cDNA sequence for contig 51.

SEQ ID NO: 538 is the determined cDNA sequence for contig 52.

SEQ ID NO: 539 is the determined cDNA sequence for contig 53.

SEQ ID NO: 540 is the determined cDNA sequence for contig 54.

SEQ ID NO: 541 is the determined cDNA sequence for contig 55.

SEQ ID NO: 542 is the determined cDNA sequence for contig 56.

SEQ ID NO: 543 is the determined cDNA sequence for contig 58.

SEQ ID NO: 544 is the determined cDNA sequence for contig 59.

SEQ ID NO: 545 is the determined cDNA sequence for contig 60.

SEQ ID NO: 546 is the determined cDNA sequence for contig 61.

SEQ ID NO: 547 is the determined cDNA sequence for contig 62.

SEQ ID NO: 548 is the determined cDNA sequence for contig 63.

SEQ ID NO: 549 is the determined cDNA sequence for contig 64.

SEQ ID NO: 550 is the determined cDNA sequence for contig 65.

SEQ ID NO: 551 is the determined cDNA sequence for contig 66.

SEQ ID NO: 552 is the determined cDNA sequence for contig 67.

SEQ ID NO: 553 is the determined cDNA sequence for contig 68.

SEQ ID NO: 554 is the determined cDNA sequence for contig 69.

SEQ ID NO: 555 is the determined cDNA sequence for contig 70.

SEQ ID NO: 556 is the determined cDNA sequence for contig 71.

SEQ ID NO: 557 is the determined cDNA sequence for contig 72.

SEQ ID NO: 558 is the determined cDNA sequence for contig 73.

SEQ ID NO: 559 is the determined cDNA sequence for contig 74.

SEQ ID NO: 560 is the determined cDNA sequence for contig 75.

SEQ ID NO: 561 is the determined cDNA sequence for contig 76.

SEQ ID NO: 562 is the determined cDNA sequence for contig 77.

SEQ ID NO: 563 is the determined cDNA sequence for contig 78.

SEQ ID NO: 564 is the determined cDNA sequence for contig 79.

SEQ ID NO: 565 is the determined cDNA sequence for contig 80.

SEQ ID NO: 566 is the determined cDNA sequence for contig 81.

SEQ ID NO: 567 is the determined cDNA sequence for contig 82.

SEQ ID NO: 568 is the determined cDNA sequence for contig 83.

SEQ ID NO: 569 is the determined cDNA sequence for clone CS1-101.

SEQ ID NO: 570 is the determined cDNA sequence for clone CS1-102.

SEQ ID NO: 571 is the determined cDNA sequence for clone CS1-104.

SEQ ID NO: 572 is the determined cDNA sequence for clone CS1-105.

SEQ ID NO: 573 is the determined 3′ cDNA sequence for clone CS1-106.

SEQ ID NO: 574 is the determined 5′ cDNA sequence for clone CS 1-106.

SEQ ID NO: 575 is the determined cDNA sequence for clone CS1-114.

SEQ ID NO: 576 is the determined cDNA sequence for clone CS1-1 18.

SEQ ID NO: 577 is the determined cDNA sequence for clone CS1-120.

SEQ ID NO: 578 is the determined cDNA sequence for clone CS1-123.

SEQ ID NO: 579 is the determined 3′ cDNA sequence for clone CS1-124.

SEQ ID NO: 580 is the determined 5′ cDNA sequence for clone CS1-124.

SEQ ID NO: 581 is the determined cDNA sequence for clone CS1-128.

SEQ ID NO: 582 is the determined cDNA sequence for clone CS1-132.

SEQ ID NO: 583 is the determined cDNA sequence for clone CS1-136.

SEQ ID NO: 584 is the determined cDNA sequence for clone CS1-137.

SEQ ID NO: 585 is the determined cDNA sequence for clone CS1-139.

SEQ ID NO: 586 is the determined cDNA sequence for clone CS1-141.

SEQ ID NO: 587 is the determined cDNA sequence for clone CS1-152.

SEQ ID NO: 588 is the determined cDNA sequence for clone CS1-154.

SEQ ID NO: 589 is the determined cDNA sequence for clone CS1-156.

SEQ ID NO: 590 is the determined cDNA sequence for clone CS1-158.

SEQ ID NO: 591 is the determined cDNA sequence for clone CS1-160.

SEQ ID NO: 592 is the determined cDNA sequence for clone CS1-168.

SEQ ID NO: 593 is the determined cDNA sequence for clone CS1-169.

SEQ ID NO: 594 is the determined cDNA sequence for clone CS1-171.

SEQ ID NO: 595 is the determined cDNA sequence for clone CS1-176.

SEQ ID NO: 596 is the determined cDNA sequence for clone CS1-178.

SEQ ID NO: 597 is the determined cDNA sequence for clone CS1-180.

SEQ ID NO: 598 is the determined cDNA sequence for clone CS1-183.

SEQ ID NO: 599 is the determined cDNA sequence for clone CS1-184.

SEQ ID NO: 600 is the determined cDNA sequence for clone CS1-187.

SEQ ID NO: 601 is the determined cDNA sequence for clone CS1-190.

SEQ ID NO: 602 is the determined cDNA sequence for clone CS1-194.

SEQ ID NO: 603 is the determined cDNA sequence for clone CS1-195.

SEQ ID NO: 604 is the determined cDNA sequence for clone CS1-196.

SEQ ID NO: 605 is the determined cDNA sequence for clone CS1-197.

SEQ ID NO: 606 is the determined cDNA sequence for clone CS1-200.

SEQ ID NO: 607 is the determined cDNA sequence for clone CS1-206.

SEQ ID NO: 608 is the determined cDNA sequence for clone CS1-207.

SEQ ID NO: 609 is the determined cDNA sequence for clone CS1-234.

SEQ ID NO: 610 is the determined cDNA sequence for clone CS1-238.

SEQ ID NO: 611 is the determined cDNA sequence for clone CS1-239.

SEQ ID NO: 612 is the determined cDNA sequence for clone CS1-243.

SEQ ID NO: 613 is the determined cDNA sequence for clone CS1-246.

SEQ ID NO: 614 is the determined cDNA sequence for clone CS1-249.

SEQ ID NO: 615 is the determined cDNA sequence for clone CS1-250.

SEQ ID NO: 616 is the determined cDNA sequence for clone CS1-252.

SEQ ID NO: 617 is the determined cDNA sequence for clone CT502.

SEQ ID NO: 618 is the determined cDNA sequence for clone CT507.

SEQ ID NO: 619 is the determined cDNA sequence for clone CT521.

SEQ ID NO: 620 is the determined cDNA sequence for clone CT544.

SEQ ID NO: 621 is the determined cDNA sequence for clone CT577.

SEQ ID NO: 622 is the determined cDNA sequence for clone CT580.

SEQ ID NO: 623 is the determined cDNA sequence for clone CT594 (also referred to by clone ID 27540).

SEQ ID NO: 624 is the determined cDNA sequence for clone CT606.

SEQ ID NO: 625 is the determined cDNA sequence for clone CT607.

SEQ ID NO: 626 is the determined cDNA sequence for clone CT599.

SEQ ID NO: 627 is the determined cDNA sequence for clone CT632.

SEQ ID NO: 628 is the determined cDNA sequence for clone 35691.

SEQ ID NO: 629 is the determined cDNA sequence for clone 35707.

SEQ ID NO: 630 is the determined cDNA sequence for clone CSE-2.

SEQ ID NO: 631 is the amino acid sequence for clone CSE-2.

SEQ ID NO: 632 is the determined cDNA sequence for clone CT2-3 .

SEQ ID NO: 633 is the determined cDNA sequence for clone CT2-6.

SEQ ID NO: 634 is the determined cDNA sequence for clone CT2-8 which shows similarity to SEQ ID NOs: 1112and 1116.

SEQ ID NO: 635 is the determined cDNA sequence for clone CT2-9.

SEQ ID NO: 636 is the determined cDNA sequence for clone CT2-12.

SEQ ID NO: 637 is the determined cDNA sequence for clone CT2-15.

SEQ ID NO: 638 is the determined cDNA sequence for clone CT2-16.

SEQ ID NO: 639 is the determined cDNA sequence for clone CT2-17.

SEQ ID NO: 640 is the determined cDNA sequence for clone CT2-19.

SEQ ID NO: 641 is the determined cDNA sequence for clone CT2-23.

SEQ ID NO: 642 is the determined cDNA sequence for clone CT2-25.

SEQ ID NO: 643 is the determined cDNA sequence for clone CT2-27.

SEQ ID NO: 644 is the determined cDNA sequence for clone CT2-35.

SEQ ID NO: 645 is the determined cDNA sequence for clone CT2-39.

SEQ ID NO: 646 is the determined cDNA sequence for clone CT2-41.

SEQ ID NO: 647 is the determined cDNA sequence for clone CT2-43.

SEQ ID NO: 648 is the determined cDNA sequence for clone CT2-44.

SEQ ID NO: 649 is the determined cDNA sequence for clone CT2-53.

SEQ ID NO: 650 is the determined cDNA sequence for clone CT2-54.

SEQ ID NO: 651 is the determined cDNA sequence for clone CT2-55.

SEQ ID NO: 652 is the determined cDNA sequence for clone CT2-57.

SEQ ID NO: 653 is the determined cDNA sequence for clone CT2-60.

SEQ ID NO: 654 is the determined cDNA sequence for clone CT2-64.

SEQ ID NO: 655 is the determined cDNA sequence for clone CT2-67.

SEQ ID NO: 656 is the determined cDNA sequence for clone CT2-68.

SEQ ID NO: 657 is the determined cDNA sequence for clone CT2-75.

SEQ ID NO: 658 is the determined cDNA sequence for clone CT2-79.

SEQ ID NO: 659 is the determined cDNA sequence for clone CT2-109.

SEQ ID NO, 660 is the determined cDNA sequence for clone CT2-112.

SEQ ID NO: 661 is the determined cDNA sequence for clone CT2-127.

SEQ ID NO: 662 is the determined cDNA sequence for clone CT2-129.

SEQ ID NO: 663 is the determined cDNA sequence for clone CT2-156.

SEQ ID NO: 664 is the determined cDNA sequence for clone CT2-162.

SEQ ID NO: 665 is the determined cDNA sequence for clone CT2-167.

SEQ ID NO: 666 is the determined cDNA sequence for clone CT2-169.

SEQ ID NO: 667 is the determined cDNA sequence for clone CT2-172.

SEQ ID NO: 668 is the determined cDNA sequence for clone CT2-173.

SEQ ID NO: 669 is the determined cDNA sequence for clone CT2-174.

SEQ ID NO: 670 is the determined cDNA sequence for clone CT2-177.

SEQ ID NO: 671 is the determined cDNA sequence for clone CT2-181.

SEQ ID NO: 672 is the determined cDNA sequence for clone CT2-191.

SEQ ID NO: 673 is the determined cDNA sequence for clone CT2-192.

SEQ ID NO: 674 is the determined cDNA sequence for clone CT2-207.

SEQ ID NO: 675 is the determined cDNA sequence for clone CT2-222.

SEQ ID NO: 676 is the determined cDNA sequence for clone CT2-223.

SEQ ID NO: 677 is the determined cDNA sequence for clone CT2-233.

SEQ ID NO: 678 is the determined cDNA sequence for clone CT2-244.

SEQ ID NO: 679 is the determined cDNA sequence for clone CT2-257.

SEQ ID NO: 680 is the determined cDNA sequence for clone CT2-279.

SEQ ID NO: 681 is the determined cDNA sequence for clone CT2-288.

SEQ ID NO: 682 is the determined cDNA sequence for clone CT2-291.

SEQ ID NO: 683 is the full-length cDNA sequence for human PAC (SEQ ID NOs: 18 and 19).

SEQ ID NO: 684 is the full-length cDNA sequence for murine homologue of human PAC (SEQ ID NO: 683).

SEQ ID NO: 685 is the predicted amino acid sequence for the clone of SEQ ID NO: 683.

SEQ ID NO: 686 is a longer determined cDNA sequence for clone CoSub-19 (SEQ ID NO: 138).

SEQ ID NO: 687 is the predicted amino acid sequence for the clone of SEQ ID NO: 686.

SEQ ID NO: 688 is the nucleotide sequence of the M13 forward primer.

SEQ ID NO: 689 is the nucleotide sequence of the M13 reverse primer.

SEQ ID NO: 690 is a longer determined cDNA sequence for C799P (SEQ ID NO: 40), showing homology to homo sapiens NADH/NADPH thyroid oxidase p138-tox mRNA.

SEQ ID NO: 691 is a longer determined cDNA sequence for C794P (SEQ ID NO: 41).

SEQ ID NO: 692 is the predicted amino acid sequence for the clone of SEQ ID NO: 690.

SEQ ID NO: 693 is the predicted amino acid sequence for the clone of SEQ ID NO: 691.

SEQ ID NO: 694 is the determined cDNA sequence for clone R0093:A03.

SEQ ID NO: 695 is the determined cDNA sequence for clone R0093:A10.

SEQ ID NO: 696 is the determined cDNA sequence for clone R0093:A11.

SEQ ID NO: 697 is the determined cDNA sequence for clone R0093:A12.

SEQ ID NO: 698 is the determined cDNA sequence for clone R0093:B03.

SEQ ID NO: 699 is the determined cDNA sequence for clone R0093:B04.

SEQ ID NO: 700 is the determined cDNA sequence for clone R0093:B09.

SEQ ID NO: 701 is the determined cDNA sequence for clone R0093:B10.

SEQ ID NO: 702 is the determined cDNA sequence for clone R0093:B11.

SEQ ID NO: 703 is the determined cDNA sequence for clone R0093:B12.

SEQ ID NO: 704 is the determined cDNA sequence for clone R0093:C01.

SEQ ID NO: 705 is the determined cDNA sequence for clone R0093:C03.

SEQ ID NO: 706 is the determined cDNA sequence for clone R0093:C04.

SEQ ID NO: 707 is the determined cDNA sequence for clone R0093:C06.

SEQ ID NO: 708 is the determined cDNA sequence for clone R0093:C08.

SEQ ID NO: 709 is the determined cDNA sequence for clone R0093:C09.

SEQ ID NO: 710 is the determined cDNA sequence for clone R0093:C10.

SEQ ID NO: 711 is the determined cDNA sequence for clone R0093:C11.

SEQ ID NO: 712 is the determined cDNA sequence for clone R0093:C12.

SEQ ID NO: 713 is the determined cDNA sequence for clone R0093:D01.

SEQ ID NO: 714 is the determined cDNA sequence for clone R0093:D02.

SEQ ID NO: 715 is the determined cDNA sequence for clone R0093:D03.

SEQ ID NO: 716 is the determined cDNA sequence for clone R0093:D04.

SEQ ID NO: 717 is the determined cDNA sequence for clone R0093:D05.

SEQ ID NO: 718 is the determined cDNA sequence for clone R0093:D06.

SEQ ID NO: 719 is the determined cDNA sequence for clone R0093:D07.

SEQ ID NO: 720 is the determined cDNA sequence for clone R0093:D08.

SEQ ID NO: 721 is the determined cDNA sequence for clone R0093:D10.

SEQ ID NO: 722 is the determined cDNA sequence for clone R0093:D11.

SEQ ID NO: 723 is the determined cDNA sequence for clone R0093:E02.

SEQ ID NO: 724 is the determined cDNA sequence for clone R0093:E03.

SEQ ID NO: 725 is the determined cDNA sequence for clone R0093:E04.

SEQ ID NO: 726 is the determined cDNA sequence for clone R0093:E06.

SEQ ID NO: 727 is the determined cDNA sequence for clone R0093:E07.

SEQ ID NO: 728 is the determined cDNA sequence for clone R0093:E08.

SEQ ID NO: 729 is the determined cDNA sequence for clone R0093:E09.

SEQ ID NO: 730 is the determined cDNA sequence for clone R0093:E10.

SEQ ID NO: 731 is the determined cDNA sequence for clone R0093:E11.

SEQ ID NO: 732 is the determined cDNA sequence for clone R0093:F02.

SEQ ID NO: 733 is the determined cDNA sequence for clone R0093:F03.

SEQ ID NO: 734 is the determined cDNA sequence for clone R0093:F04.

SEQ ID NO: 735 is the determined cDNA sequence for clone R0093:F05.

SEQ ID NO: 736 is the determined cDNA sequence for clone R0093:F06.

SEQ ID NO: 737 is the determined cDNA sequence for clone R0093:F08.

SEQ ID NO: 738 is the determined cDNA sequence for clone R0093:F09.

SEQ ID NO: 739 is the determined cDNA sequence for clone R0093:F10.

SEQ ID NO: 740 is the determined cDNA sequence for clone R0093:F12.

SEQ ID NO: 741 is the determined cDNA sequence for clone R0093:G01.

SEQ ID NO: 742 is the determined cDNA sequence for clone R0093:G03.

SEQ ID NO: 743 is the determined cDNA sequence for clone R0093:G04.

SEQ ID NO: 744 is the determined cDNA sequence for clone R0093:G06.

SEQ ID NO: 745 is the determined cDNA sequence for clone R0093:G07.

SEQ ID NO: 746 is the determined cDNA sequence for clone R0093:G08.

SEQ ID NO: 747 is the determined cDNA sequence for clone R0093:G09.

SEQ ID NO: 748 is the determined cDNA sequence for clone R0093:G10.

SEQ ID NO: 749 is the determined cDNA sequence for clone R0093:G11.

SEQ ID NO: 750 is the determined cDNA sequence for clone R0093:G12.

SEQ ID NO: 751 is the determined cDNA sequence for clone R0093:H02.

SEQ ID NO: 752 is the determined cDNA sequence for clone R0093:H03.

SEQ ID NO: 753 is the determined cDNA sequence for clone R0093:H04.

SEQ ID NO: 754 is the determined cDNA sequence for clone R0093:H05.

SEQ ID NO: 755 is the determined cDNA sequence for clone R0093:H07.

SEQ ID NO: 756 is the determined cDNA sequence for clone R0093:H08.

SEQ ID NO: 757 is the determined cDNA sequence for clone R0093:H09.

SEQ ID NO: 758 is the determined cDNA sequence for clone R0093:H10.

SEQ ID NO: 759 is the determined cDNA sequence for clone R0093:H11.

SEQ ID NO: 760 is the determined cDNA sequence for clone R0094:A03.

SEQ ID NO: 761 is the determined cDNA sequence for clone R0094:A05.

SEQ ID NO: 762 is the determined cDNA sequence for clone R0094:A06.

SEQ ID NO: 763 is the determined cDNA sequence for clone R0094:A07.

SEQ ID NO: 764 is the determined cDNA sequence for clone R0094:A09.

SEQ ID NO: 765 is the determined cDNA sequence for clone R0094:A10.

SEQ ID NO: 766 is the determined cDNA sequence for clone R0094:A12.

SEQ ID NO: 767 is the determined cDNA sequence for clone R0094:B03.

SEQ ID NO: 768 is the determined cDNA sequence for clone R0094:B06.

SEQ ID NO: 769 is the determined cDNA sequence for clone R0094:B08.

SEQ ID NO: 770 is the determined cDNA sequence for clone R0094:B11.

SEQ ID NO: 771 is the determined cDNA sequence for clone R0094:B12.

SEQ ID NO: 772 is the determined cDNA sequence for clone R0094:C01.

SEQ ID NO: 773 is the determined cDNA sequence for clone R0094:C02.

SEQ ID NO: 774 is the determined cDNA sequence for clone R0094:C03.

SEQ ID NO: 775 is the determined cDNA sequence for clone R0094:C05.

SEQ ID NO: 776 is the determined cDNA sequence for clone R0094:C06.

SEQ ID NO: 777 is the determined cDNA sequence for clone R0094:C08.

SEQ ID NO: 778 is the determined cDNA sequence for clone R0094:C09.

SEQ ID NO: 779 is the determined cDNA sequence for clone R0094:C10.

SEQ ID NO: 780 is the determined cDNA sequence for clone R0094:C11.

SEQ ID NO: 781 is the determined cDNA sequence for clone R0094:C12.

SEQ ID NO: 782 is the determined cDNA sequence for clone R0094:C01.

SEQ ID NO: 783 is the determined cDNA sequence for clone R0094:D02.

SEQ ID NO: 784 is the determined cDNA sequence for clone R0094:D03.

SEQ ID NO: 785 is the determined cDNA sequence for clone R0094:D04.

SEQ ID NO: 786 is the determined cDNA sequence for clone R0094:D05.

SEQ ID NO: 787 is the determined cDNA sequence for clone R0094:D07.

SEQ ID NO: 788 is the determined cDNA sequence for clone R0094:D08.

SEQ ID NO: 789 is the determined cDNA sequence for clone R0094:D09.

SEQ ID NO: 790 is the determined cDNA sequence for clone R0094:D10.

SEQ ID NO: 791 is the determined cDNA sequence for clone R0094:D12.

SEQ ID NO: 792 is the determined cDNA sequence for clone R0094:E01.

SEQ ID NO: 793 is the determined cDNA sequence for clone R0094:E02.

SEQ ID NO: 794 is the determined cDNA sequence for clone R0094:E03.

SEQ ID NO: 795 is the determined cDNA sequence for clone R0094:E05.

SEQ ID NO: 796 is the determined cDNA sequence for clone R0094:E06.

SEQ ID NO: 797 is the determined cDNA sequence for clone R0094:E07.

SEQ ID NO: 798 is the determined cDNA sequence for clone R0094:E08.

SEQ ID NO: 799 is the determined cDNA sequence for clone R0094:E09.

SEQ ID NO: 800 is the determined cDNA sequence for clone R0094:E10.

SEQ ID NO: 801 is the determined cDNA sequence for clone R0094:E11.

SEQ ID NO: 802 is the determined cDNA sequence for clone R0094:E12.

SEQ ID NO: 803 is the determined cDNA sequence for clone R0094:F01.

SEQ ID NO: 804 is the determined cDNA sequence for clone R0094:F03.

SEQ ID NO: 805 is the determined cDNA sequence for clone R0094:F05.

SEQ ID NO: 806 is the determined cDNA sequence for clone R0094:F06.

SEQ ID NO: 807 is the determined cDNA sequence for clone R0094:F07.

SEQ ID NO: 808 is the determined cDNA sequence for clone R0094:F08.

SEQ ID NO: 809 is the determined cDNA sequence for clone R0094:F09.

SEQ ID NO: 810 is the determined cDNA sequence for clone R0094:F10.

SEQ ID NO: 811 is the determined cDNA sequence for clone R0094:F11.

SEQ ID NO: 812 is the determined cDNA sequence for clone R0094:F12.

SEQ ID NO: 813 is the determined cDNA sequence for clone R0094:G02.

SEQ ID NO: 814 is the determined cDNA sequence for clone R0094:G03.

SEQ ID NO: 815 is the determined cDNA sequence for clone R0094:G04.

SEQ ID NO: 816 is the determined cDNA sequence for clone R0094:G06.

SEQ ID NO: 817 is the determined cDNA sequence for clone R0094:G07.

SEQ ID NO: 818 is the determined cDNA sequence for clone R0094:G08.

SEQ ID NO: 819 is the determined cDNA sequence for clone R0094:G10.

SEQ ID NO: 820 is the determined cDNA sequence for clone R0094:G11.

SEQ ID NO: 821 is the determined cDNA sequence for clone R0094:G12.

SEQ ID NO: 822 is the determined cDNA sequence for clone R0094:H01.

SEQ ID NO: 823 is the determined cDNA sequence for clone R0094:H03.

SEQ ID NO: 824 is the determined cDNA sequence for clone R0094:H04.

SEQ ID NO: 825 is the determined cDNA sequence for clone R0094:H05.

SEQ ID NO: 826 is the determined cDNA sequence for clone R0094:H06.

SEQ ID NO: 827 is the determined cDNA sequence for clone R0094:H08.

SEQ ID NO: 828 is the determined cDNA sequence for clone R0094:H09.

SEQ ID NO: 829 is the determined cDNA sequence for clone R0094:H10.

SEQ ID NO: 830 is the determined cDNA sequence for clone R0094:H11.

SEQ ID NO: 831 is the determined cDNA sequence for clone R0095:A03.

SEQ ID NO: 832 is the determined cDNA sequence for clone R0095:A06.

SEQ ID NO: 833 is the determined cDNA sequence for clone R0095:A07.

SEQ ID NO: 834 is the determined cDNA sequence for clone R0095:B01.

SEQ ID NO: 835 is the determined cDNA sequence for clone R0095:B02.

SEQ ID NO: 836 is the determined cDNA sequence for clone R0095:B03.

SEQ ID NO: 837 is the determined cDNA sequence for clone R0095:B04.

SEQ ID NO: 838 is the determined cDNA sequence for clone R0095:B05.

SEQ ID NO: 839 is the determined cDNA sequence for clone R0095:B06.

SEQ ID NO: 840 is the determined cDNA sequence for clone R0095:B10.

SEQ ID NO: 841 is the determined cDNA sequence for clone R0095:B11.

SEQ ID NO: 842 is the determined cDNA sequence for clone R0095:B12.

SEQ ID NO: 843 is the determined cDNA sequence for clone R0095:C01.

SEQ ID NO: 844 is the determined cDNA sequence for clone R0095:C03.

SEQ ID NO: 845 is the determined cDNA sequence for clone R0095:C04.

SEQ ID NO: 846 is the determined cDNA sequence for clone R0095:C05.

SEQ ID NO: 847 is the determined cDNA sequence for clone R0095:C06.

SEQ ID NO: 848 is the determined cDNA sequence for clone R0095:C07.

SEQ ID NO: 849 is the determined cDNA sequence for clone R0095:C08.

SEQ ID NO: 850 is the determined cDNA sequence for clone R0095:C10.

SEQ ID NO: 851 is the determined cDNA sequence for clone R0095:C12.

SEQ ID NO: 852 is the determined cDNA sequence for clone R0095:D01.

SEQ ID NO: 853 is the determined cDNA sequence for clone R0095:D03.

SEQ ID NO: 854 is the determined cDNA sequence for clone R0095:D04.

SEQ ID NO: 855 is the determined cDNA sequence for clone R0095:D06.

SEQ ID NO: 856 is the determined cDNA sequence for clone R0095:D07.

SEQ ID NO: 857 is the determined cDNA sequence for clone R0095:D08.

SEQ ID NO: 858 is the determined cDNA sequence for clone R0095:D09.

SEQ ID NO: 859 is the determined cDNA sequence for clone R0095:D11.

SEQ ID NO: 860 is the determined cDNA sequence for clone R0095:D12.

SEQ ID NO: 861 is the determined cDNA sequence for clone R0095:E01.

SEQ ID NO: 862 is the determined cDNA sequence for clone R0095:E02.

SEQ ID NO: 863 is the determined cDNA sequence for clone R0095:E04.

SEQ ID NO: 864 is the determined cDNA sequence for clone R0095:E05.

SEQ ID NO: 865 is the determined cDNA sequence for clone R0095:E06.

SEQ ID NO: 866 is the determined cDNA sequence for clone R0095:E07.

SEQ ID NO: 867 is the determined cDNA sequence for clone R0095:E08.

SEQ ID NO: 868 is the determined cDNA sequence for clone R0095:E11.

SEQ ID NO: 869 is the determined cDNA sequence for clone R0095:E12.

SEQ ID NO: 870 is the determined cDNA sequence for clone R0095:F01.

SEQ ID NO: 871 is the determined cDNA sequence for clone R0095:F03.

SEQ ID NO: 872 is the determined cDNA sequence for clone R0095:F06.

SEQ ID NO: 873 is the determined cDNA sequence for clone R0095:F10.

SEQ ID NO: 874 is the determined cDNA sequence for clone R0095:F11.

SEQ ID NO: 875 is the determined cDNA sequence for clone R0095:G02.

SEQ ID NO: 876 is the determined cDNA sequence for clone R0095:G03.

SEQ ID NO: 877 is the determined cDNA sequence for clone R0095:G04.

SEQ ID NO: 878 is the determined cDNA sequence for clone R0095:G08.

SEQ ID NO: 879 is the determined cDNA sequence for clone R0095:G09.

SEQ ID NO: 880 is the determined cDNA sequence for clone R0095:G10.

SEQ ID NO: 881 is the determined cDNA sequence for clone R0095:H01.

SEQ ID NO: 882 is the determined cDNA sequence for clone R0095:H02.

SEQ ID NO: 883 is the determined cDNA sequence for clone R0095:H14.

SEQ ID NO: 884 is the determined cDNA sequence for clone R0095:H06.

SEQ ID NO: 885 is the determined cDNA sequence for clone R0095:H07.

SEQ ID NO: 886 is the determined cDNA sequence for clone R0095:H09.

SEQ ID NO: 887 is the determined cDNA sequence for clone R0096:A02.

SEQ ID NO: 888 is the determined cDNA sequence for clone R0096:A08.

SEQ ID NO: 889 is the determined cDNA sequence for clone R0096:A09.

SEQ ID NO: 890 is the determined cDNA sequence for clone R0096:A10.

SEQ ID NO: 891 is the determined cDNA sequence for clone R0096:A11.

SEQ ID NO: 892 is the determined cDNA sequence for clone R0096:A12.

SEQ ID NO: 893 is the determined cDNA sequence for clone R0096:B02.

SEQ ID NO: 894 is the determined cDNA sequence for clone R0096:B03.

SEQ ID NO: 895 is the determined cDNA sequence for clone R0096:B04.

SEQ ID NO: 896 is the determined cDNA sequence for clone R0096:B05.

SEQ ID NO: 897 is the determined cDNA sequence for clone R0096:B06.

SEQ ID NO: 898 is the determined cDNA sequence for clone R0096:B07.

SEQ ID NO: 899 is the determined cDNA sequence for clone R0096:B08.

SEQ ID NO: 900 is the determined cDNA sequence for clone R0096:B09.

SEQ ID NO: 901 is the determined cDNA sequence for clone R0096:B10.

SEQ ID NO: 902 is the determined cDNA sequence for clone R0096:B11.

SEQ ID NO: 903 is the determined cDNA sequence for clone R0096:B12.

SEQ ID NO: 904 is the determined cDNA sequence for clone R0096:C01.

SEQ ID NO: 905 is the determined cDNA sequence for clone R0096:C03.

SEQ ID NO: 906 is the determined cDNA sequence for clone R0096:C04.

SEQ ID NO: 907 is the determined cDNA sequence for clone R0096:C05.

SEQ ID NO: 908 is the determined cDNA sequence for clone R0096:C06.

SEQ ID NO: 909 is the determined cDNA sequence for clone R0096:C07.

SEQ ID NO: 910 is the determined cDNA sequence for clone R0096:C08.

SEQ ID NO: 911 is the determined cDNA sequence for clone R0096:C09.

SEQ ID NO: 912 is the determined cDNA sequence for clone R0096:C10.

SEQ ID NO: 913 is the determined cDNA sequence for clone R0096:C11.

SEQ ID NO: 914 is the determined cDNA sequence for clone R0096:C12.

SEQ ID NO: 915 is the determined cDNA sequence for clone R0096:D01.

SEQ ID NO: 916 is the determined cDNA sequence for clone R0096:D02.

SEQ ID NO: 917 is the determined cDNA sequence for clone R0096:D03.

SEQ ID NO: 918 is the determined cDNA sequence for clone R0096:D04.

SEQ ID NO: 919 is the determined cDNA sequence for clone R0096:D05.

SEQ ID NO: 920 is the determined cDNA sequence for clone R0096:D08.

SEQ ID NO: 921 is the determined cDNA sequence for clone R0096:D09.

SEQ ID NO: 922 is the determined cDNA sequence for clone R0096:D10.

SEQ ID NO: 923 is the determined cDNA sequence for clone R0096:D12.

SEQ ID NO: 924 is the determined cDNA sequence for clone R0096:E01.

SEQ ID NO: 925 is the determined cDNA sequence for clone R0096:E02.

SEQ ID NO: 926 is the determined cDNA sequence for clone R0096:E03.

SEQ ID NO: 927 is the determined cDNA sequence for clone R0096:E04.

SEQ ID NO: 928 is the determined cDNA sequence for clone R0096:E05.

SEQ ID NO: 929 is the determined cDNA sequence for clone R0096:E06.

SEQ ID NO: 930 is the determined cDNA sequence for clone R0096:E08.

SEQ ID NO: 931 is the determined cDNA sequence for clone R0096:E09.

SEQ ID NO: 932 is the determined cDNA sequence for clone R0096:E10.

SEQ ID NO: 933 is the determined cDNA sequence for clone R0096:E11.

SEQ ID NO: 934 is the determined cDNA sequence for clone R0096:F12.

SEQ ID NO: 935 is the determined cDNA sequence for clone R0096:F01.

SEQ ID NO: 936 is the determined cDNA sequence for clone R0096:F02.

SEQ ID NO: 937 is the determined cDNA sequence for clone R0096:F03.

SEQ ID NO: 938 is the determined cDNA sequence for clone R0096:F04.

SEQ ID NO: 939 is the determined cDNA sequence for clone R0096:F05.

SEQ ID NO: 940 is the determined cDNA sequence for clone R0096:F07.

SEQ ID NO: 941 is the determined cDNA sequence for clone R0096:F10.

SEQ ID NO: 942 is the determined cDNA sequence for clone R0096:F11.

SEQ ID NO: 943 is the determined cDNA sequence for clone R0096:G01.

SEQ ID NO: 944 is the determined cDNA sequence for clone R0096:G03.

SEQ ID NO: 945 is the determined cDNA sequence for clone R0096:G04.

SEQ ID NO: 946 is the determined cDNA sequence for clone R0096:G05.

SEQ ID NO: 947 is the determined cDNA sequence for clone R0096:G06.

SEQ ID NO: 948 is the determined cDNA sequence for clone R0096:G07.

SEQ ID NO: 949 is the determined cDNA sequence for clone R0096:G09.

SEQ ID NO: 950 is the determined cDNA sequence for clone R0096:G10.

SEQ ID NO: 951 is the determined cDNA sequence for clone R0096:G12.

SEQ ID NO: 952 is the determined cDNA sequence for clone R0096:H01.

SEQ ID NO: 953 is the determined cDNA sequence for clone R0096:H02.

SEQ ID NO: 954 is the determined cDNA sequence for clone R0096:H03.

SEQ ID NO: 955 is the determined cDNA sequence for clone R0096:H07.

SEQ ID NO: 956 is the determined cDNA sequence for clone R0096:H08.

SEQ ID NO: 957 is the determined cDNA sequence for clone R0097:A05

SEQ ID NO: 958 is the determined cDNA sequence for clone R0097:A06.

SEQ ID NO: 959 is the determined cDNA sequence for clone R0097:A10.

SEQ ID NO: 960 is the determined cDNA sequence for clone R0097:A11.

SEQ ID NO: 961 is the determined cDNA sequence for clone R0097:B01.

SEQ ID NO: 962 is the determined cDNA sequence for clone R0097:B03.

SEQ ID NO: 963 is the determined cDNA sequence for clone R0097:B04.

SEQ ID NO: 964 is the determined cDNA sequence for clone R0097:B05.

SEQ ID NO: 965 is the determined cDNA sequence for clone R0097:B06.

SEQ ID NO: 966 is the determined cDNA sequence for clone R0097:B07.

SEQ ID NO: 967 is the determined cDNA sequence for clone R0097:B11.

SEQ ID NO: 968 is the determined cDNA sequence for clone R0097:C01.

SEQ ID NO: 969 is the determined cDNA sequence for clone R0097:C02.

SEQ ID NO: 970 is the determined cDNA sequence for clone R0097:C03.

SEQ ID NO: 971 is the determined cDNA sequence for clone R0097:C04.

SEQ ID NO: 972 is the determined cDNA sequence for clone R0097:C05.

SEQ ID NO: 973 is the determined cDNA sequence for clone R0097:C07.

SEQ ID NO: 974 is the determined cDNA sequence for clone R0097:C08.

SEQ ID NO: 975 is the determined cDNA sequence for clone R0097:C09.

SEQ ID NO: 976 is the determined cDNA sequence for clone R0097:C10.

SEQ ID NO: 977 is the determined cDNA sequence for clone R0097:D10.

SEQ ID NO: 978 is the determined cDNA sequence for clone R0097:D08.

SEQ ID NO: 979 is the determined cDNA sequence for clone R0097:E02.

SEQ ID NO: 980 is the determined cDNA sequence for clone R0097:E09.

SEQ ID NO: 981 is the determined cDNA sequence for clone R0097:E11.

SEQ ID NO: 982 is the determined cDNA sequence for clone R0097:F01.

SEQ ID NO: 983 is the determined cDNA sequence for clone R0097:F11.

SEQ ID NO: 984 is the determined cDNA sequence for clone R0097:G01.

SEQ ID NO: 985 is the determined cDNA sequence for clone R0097:G11.

SEQ ID NO: 986 is the determined cDNA sequence for clone R0097:G12.

SEQ ID NO: 987 is the determined cDNA sequence for clone R0097:H01.

SEQ ID NO: 988 is the determined cDNA sequence for clone R0097:H02.

SEQ ID NO: 989 is the determined cDNA sequence for clone R0097:H04.

SEQ ID NO: 990 is the determined cDNA sequence for clone R0097:H06.

SEQ ID NO: 991 is the determined cDNA sequence for clone R0097:H07.

SEQ ID NO: 992 is the determined cDNA sequence for clone R0097:H09.

SEQ ID NO: 993 is the determined cDNA sequence for clone R0097:H11.

SEQ ID NO: 994 is the determined cDNA sequence for clone R0098:A03.

SEQ ID NO: 995 is the determined cDNA sequence for clone R0098:A05.

SEQ ID NO: 996 is the determined cDNA sequence for clone R0098:A06.

SEQ ID NO: 997 is the determined cDNA sequence for clone R0098:A10.

SEQ ID NO: 998 is the determined cDNA sequence for clone R0098:A12.

SEQ ID NO: 999 is the determined cDNA sequence for clone R0098:B01.

SEQ ID NO: 1000 is the determined cDNA sequence for clone R0098:B02.

SEQ ID NO: 1001 is the determined cDNA sequence for clone R0098:B05.

SEQ ID NO: 1002 is the determined cDNA sequence for clone R0098:B06.

SEQ ID NO: 1003 is the determined cDNA sequence for clone R0098:B10.

SEQ ID NO: 1004 is the determined cDNA sequence for clone R0098:C03.

SEQ ID NO: 1005 is the determined cDNA sequence for clone R0098:C04.

SEQ ID NO: 1006 is the determined cDNA sequence for clone R0098:C05.

SEQ ID NO: 1007 is the determined cDNA sequence for clone R0098:C10.

SEQ ID NO: 1008 is the determined cDNA sequence for clone R0098:C11.

SEQ ID NO: 1009 is the determined cDNA sequence for clone R0098:D01.

SEQ ID NO: 1010 is the determined cDNA sequence for clone R0098:D02.

SEQ ID NO: 1011 is the determined cDNA sequence for clone R0098:D07.

SEQ ID NO: 1012 is the determined cDNA sequence for clone R0098:D08.

SEQ ID NO: 1013 is the determined cDNA sequence for clone R0098:D09.

SEQ ID NO: 1014 is the determined cDNA sequence for clone R0098:D10.

SEQ ID NO: 1015 is the determined cDNA sequence for clone R0098:D11.

SEQ ID NO: 1016 is the determined cDNA sequence for clone R0098:D12.

SEQ ID NO: 1017 is the determined cDNA sequence for clone R0098:E01.

SEQ ID NO: 1018 is the determined cDNA sequence for clone R0098:E04.

SEQ ID NO: 1019 is the determined cDNA sequence for clone R0098:E05.

SEQ ID NO: 1020 is the determined cDNA sequence for clone R0098:E06.

SEQ ID NO: 1021 is the determined cDNA sequence for clone R0098:E07.

SEQ ID NO: 1022 is the determined cDNA sequence for clone R0098:E11.

SEQ ID NO: 1023 is the determined cDNA sequence for clone R0098:F04.

SEQ ID NO: 1024 is the determined cDNA sequence for clone R0098:F05.

SEQ ID NO: 1025 is the determined cDNA sequence for clone R0098:F06.

SEQ ID NO: 1026 is the determined cDNA sequence for clone R0098:F07.

SEQ ID NO: 1027 is the determined cDNA sequence for clone R0098:F08.

SEQ ID NO: 1028 is the determined cDNA sequence for clone R0098:F09.

SEQ ID NO: 1029 is the determined cDNA sequence for clone R0098:F10.

SEQ ID NO: 1030 is the determined cDNA sequence for clone R0098:F11.

SEQ ID NO: 1031 is the determined cDNA sequence for clone R0098:F12.

SEQ ID NO: 1032 is the determined cDNA sequence for clone R0098:G02.

SEQ ID NO: 1033 is the determined cDNA sequence for clone R0098:G03.

SEQ ID NO: 1034 is the determined cDNA sequence for clone R0098:G05.

SEQ ID NO: 1035 is the determined cDNA sequence for clone R0098:G06.

SEQ ID NO: 1036 is the determined cDNA sequence for clone R0098:G07.

SEQ ID NO: 1037 is the determined cDNA sequence for clone R0098:G08.

SEQ ID NO: 1038 is the determined cDNA sequence for clone R0098:G09.

SEQ ID NO: 1039 is the determined cDNA sequence for clone R0098:G10.

SEQ ID NO: 1040 is the determined cDNA sequence for clone R0098:G11.

SEQ ID NO: 1041 is the determined cDNA sequence for clone R0098:G12.

SEQ ID NO: 1042 is the determined cDNA sequence for clone R0098:H02.

SEQ ID NO: 1043 is the determined cDNA sequence for clone R0098:H03.

SEQ ID NO: 1044 is the determined cDNA sequence for clone R0098:H04.

SEQ ID NO: 1045 is the determined cDNA sequence for clone R0098:H05.

SEQ ID NO: 1046 is the determined cDNA sequence for clone R0098:H07.

SEQ ID NO: 1047 is the determined cDNA sequence for clone R0098:H08.

SEQ ID NO: 1048 is the determined cDNA sequence for clone R0098:H11.

SEQ ID NO: 1049 is the determined cDNA sequence for clone C878P which shows sequence similarity to homo sapiens cDNA FLJ10884 fis, clone NT2RP4001950 and homo sapiens cDNA FLJ11111 fis, clone PLACE1005923.

SEQ ID NO: 1050 is the determined cDNA sequence for clone C882P which shows sequence similarity to homo sapiens cDNA FLJ20116 fis, clone COLO 5655 and homo sapiens cDNA FLJ20740 fis, clone HEP07118.

SEQ ID NO: 1051 is the determined cDNA sequence for clone C883P which shows sequence similarity to human homeobox protein Cdx2 mRNA.

SEQ ID NO: 1052 is the determined cDNA sequence for clone C884P which shows sequence similarity to human TM4SF3 (aka, CO-029).

SEQ ID NO: 1053 is the determined cDNA sequence for clone C886P which shows sequence similarity to human secretory protein (P1.B) mRNA and homo sapiens trefoil factor 3 (intestinal) (TFF3) mRNA.

SEQ ID NO: 1054 is the determined cDNA sequence for clone C892P which shows sequence similarity to human galectin-4 mRNA.

SEQ ID NO: 1055 is the determined cDNA sequence for clone C900P which shows sequence similarity to homo sapiens mucin 11 (MUC11) mRNA.

SEQ ID NO: 1056 is the determined cDNA sequence for clone C902P which shows sequence similarity to homo sapiens calcium-dependent chloride channel-1 (hCLCA1) mRNA.

SEQ ID NO: 1057 is the determined cDNA sequence for clone C903P which shows sequence similarity to homo sapiens transmembrane mucin 12 (MUC12) mRNA.

SEQ ID NO: 1058 is the determined cDNA sequence for clone C899P which shows sequence similarity to homo sapiens intestinal mucin (MUC2) mRNA.

SEQ ID NO: 1059 is the predicted amino acid sequence for the clone of SEQ ID NO: 1049.

SEQ ID NO: 1060 is the predicted amino acid sequence for the clone of SEQ ID NO: 1050.

SEQ ID NO: 1061 is the predicted amino acid sequence for the clone of SEQ ID NO: 1051.

SEQ ID NO: 1062 is the predicted amino acid sequence for the clone of SEQ ID NO: 1052.

SEQ ID NO: 1063 is the predicted amino acid sequence for the clone of SEQ ID NO: 1053.

SEQ ID NO: 1064 is the predicted amino acid sequence for the clone of SEQ ID NO: 1054.

SEQ ID NO: 1065 is the predicted amino acid sequence for the clone of SEQ ID NO: 1055.

SEQ ID NO: 1066 is the predicted amino acid sequence for the clone of SEQ ID NO: 1056.

SEQ ID NO: 1067 is the predicted amino acid sequence for the clone of SEQ ID NO: 1057.

SEQ ID NO: 1068 is the predicted amino acid sequence for the clone of SEQ ID NO: 1058.

SEQ ID NO: 1069 is the full length nucleotide sequence for clone CS1-152 (C880P, C887P).

SEQ ID NO: 1070 is the predicted amino acid sequence for the clone of SEQ ID NO: 1069.

SEQ ID NO: 1071 is the cDNA sequence for human colon specific gene (geneseq X03195) identified from a computer search of the public geneseq database and which shows similarity to clone C880P.

SEQ ID NO: 1072 is the cDNA sequence for human protein comprising secretory signal nucleotide sequence 3 (geneseq V29035) identified from a computer search of the public geneseq database and which shows similarity to clone C880P.

SEQ ID NO: 1073 is the cDNA sequence for open reading frame human protein comprising secretory signal 3 (geneseq V29036) identified from a computer search of the public geneseq database and which shows similarity to clone C880P.

SEQ ID NO: 1074 is the cDNA sequence for human colon specific protein cDNA (geneseq T51784) identified from a computer search of the public geneseq database and which shows similarity to clone C880P.

SEQ ID NO: 1075 is the cDNA sequence for human Reg 1-gamma protein (geneseq V29156) identified from a computer search of the public geneseq database and which shows similarity to clone C880P.

SEQ ID NO: 1076 is the cDNA sequence for human intestinal peptide-associated transporter HPT-1 mRNA, complete cds and homo sapiens mRNA for L1-cadherin (geneseq X18166) identified from a computer search of the public geneseq database and which shows similarity to clone C888P.

SEQ ID NO: 1077 is the amino acid sequence of geneseq record W12691 which shows sequence similarity to clone C880P.

SEQ ID NO: 1078 is the amino acid sequence of geneseq record W37866 which shows sequence similarity to clone C880P.

SEQ ID NO: 1079 is the amino acid sequence of geneseq record W37929 which shows sequence similarity to clone C880P.

SEQ ID NO: 1080 is the amino acid sequence of geneseq record W84274 which shows sequence similarity to clone C880P.

SEQ ID NO: 1081 is the amino acid sequence of geneseq record W740898 which shows sequence similarity to clone C888P.

SEQ ID NO: 1082 is the determined cDNA sequence for clone 27540

SEQ ID NO: 1083 is the predicted amino acid sequence of clone 27540 (SEQ ID NO: 1082).

SEQ ID NO: 1084 is the determined cDNA sequence for Ra12-C884P-PCRX2.

SEQ ID NO: 1085 the predicted amino acid sequence for Ra12-C884P (SEQ ID NO: 1084).

SEQ ID NO: 1086 the determined cDNA sequence for R.C888P.

SEQ ID NO: 1087 the predicted amino acid sequence for R.C888P (SEQ ID NO: 1086).

SEQ ID NO: 1088 is the PCR primer 080300-A (Primer Identifier 7839) for amplification of C884.

SEQ ID NO: 1089 is the PCR primer 080300-B (Primer Identifier 7840) for amplification of C884.

SEQ ID NO: 1090 is the sense PCR Primer -080300-C (Primer ID7841) for amplification of C888P.

SEQ ID NO: 1091 is the antisense PCR Primer -080300-D (Primer ID7842) for amplification of C888P.

SEQ ID NO: 1092 is the determined cDNA sequence for the RECC gene.

SEQ ID NO: 1093 is the predicted protein sequence encoded by SEQ ID NO: 1092.

SEQ ID NO. 1094 is the full length C799P/THOX2 cDNA sequence.

SEQ ID NO: 1095 is the full length C799P/THOX2 amino acid sequence that is the amino acid sequence predicted by the ORF contained in SEQ ID NO: 1094.

SEQ ID NO: 1096 is the full length cDNA sequence for Galectin-4, which shows similarity to clone CT-53 (C892P).

SEQ ID NO: 1097 is the full length cDNA sequence for Human SurfaceMarker 1 GPI-anchored, which shows similarity to clone CT-126 (25527).

SEQ ID NO: 1098 is the full length cDNA sequence for hu.XAG homolog Anterior Gradient Protein, which shows similarity to clone CT-140 (25537).

SEQ ID NO: 1099 is the full length cDNA sequence for hu glucose phosphate isomerase (GPI), which shows similarity to clone CT-148 (25544).

SEQ ID NO: 1100 is the determined cDNA sequence for clone CT2-283 (41103).

SEQ ID NO: 1101 is the full length cDNA sequence for APG-2, which shows similarity to clones CT2-136 (41099) and CT-17 (24115).

SEQ ID NO: 1102 is the predicted full length protein sequence for APG-2, which shows similarity to clones CT2-136 (41099) and CT-17 (24115).

SEQ ID NO: 1103 is the full length cDNA sequence for human squalene epoxidase, which shows similarity to clone CS1-104 (31409).

SEQ ID NO: 1104 is the full length cDNA sequence for human Mad2, which shows similarity to clone CS1-106 (31364).

SEQ ID NO: 1105 is the full length cDNA sequence for epithelial-specific transcription factor, which shows similarity to clone CS1-123 (31396).

SEQ ID NO: 1106 is the full length cDNA sequence for KSA, Adenocarcinoma-associated antigen, which shows similarity to clone CS1-160 (32222).

SEQ ID NO: 1107 is the predicted protein sequence encoded by SEQ ID NO: 1103.

SEQ ID NO: 1108 is the predicted protein sequence encoded by SEQ ID NO: 1104.

SEQ ID NO: 1109 is the predicted protein sequence encoded by SEQ ID NO: 1105.

SEQ ID NO: 1109 is the predicted protein sequence encoded by SEQ ID NO: 1106.

SEQ ID NO: 1111 is the full length cDNA sequence for Hu.Keratin19, which shows similarity to clone CT2-167.

SEQ ID NO: 1112 is the full length cDNA sequence for Hu.IgG Fc-binding protein (FC(GAMMA)BP), which shows similarity to clone CT2-147 (41100).

SEQ ID NO: 1113 is the full length cDNA sequence for murine Valosin-containing protein, which shows similarity to clone CT2-222 (39848).

SEQ ID NO: 1114 is the full length cDNA sequence for human Valosin-containing protein, which shows similarity to clone CT2-222 (39848).

SEQ ID NO: 1115 is the predicted protein sequence encoded by SEQ ID NO: 1111.

SEQ ID NO: 1116 is the predicted protein sequence encoded by SEQ ID NO: 1112.

SEQ ID NO: 1117 is the predicted protein sequence encoded by SEQ ID NO: 1113.

SEQ ID NO: 1118 is the predicted protein sequence encoded by SEQ ID NO: 1114.

SEQ ID NO: 1119 is the determined cDNA sequence for MAPS-C884P.

SEQ ID NO: 1120 is the determined cDNA sequence C884P-HisTag.

SEQ ID NO: 1121 is the predicted protein sequence encoded by SEQ ID NO: 1119.

SEQ ID NO: 1122 is the predicted protein sequence encoded by SEQ ID NO: 1120.

### DETAILED DESCRIPTION OF THE INVENTION

As noted above, the present invention is generally directed to compositions and methods for the therapy and diagnosis of cancer, such as colon cancer. The compositions described herein may include colon tumor polypeptides, polynucleotides encoding such polypeptides, binding agents such as antibodies, antigen presenting cells (APCs) and/or immune system cells (e.g., T cells). Polypeptides of the present invention generally comprise at least a portion (such as an immunogenic portion) of a colon tumor protein or a variant thereof. A “colon tumor protein” is a protein that is expressed in colon tumor cells at a level that is at least two fold, and preferably at least five fold, greater than the level of expression in a normal tissue, as determined using a representative assay provided herein. Certain colon tumor proteins are tumor proteins that react detectably (within an immunoassay, such as an ELISA or Western blot) with antisera of a patient afflicted with colon cancer. Polynucleotides of the subject invention generally comprise a DNA or RNA sequence that encodes all or a portion of such a polypeptide, or that is complementary to such a sequence. Antibodies are generally immune system proteins, or antigen-binding fragments thereof, that are capable of binding to a polypeptide as described above. Antigen presenting cells include dendritic cells, macrophages, monocytes, fibroblasts and B-cells that express a polypeptide as described above. T cells that may be employed within such compositions are generally T cells that are specific for a polypeptide as described above.

The present invention is based on the discovery of human colon tumor proteins. Sequences of polynucleotides encoding specific tumor proteins are provided in SEQ ID NO: 1-121, 123-197, 205-630, 632-684, 686, 690-691, 694-1058, 1069, 1071-1076, 1082, 1084, 1086, 1092, 1094, 1096-1101, 1103-1106, 1111-1114, 1119, and 1120.

Colon Tumor Protein Polynucleotides

Any polynucleotide that encodes a colon tumor protein or a portion or other variant thereof as described herein is encompassed by the present invention. Preferred polynucleotides comprise at least 15 consecutive nucleotides, preferably at least 30 consecutive nucleotides and more preferably at least 45 consecutive nucleotides, that encode a portion of a colon tumor protein. More preferably, a polynucleotide encodes an immunogenic portion of a colon tumor protein. Polynucleotides complementary to any such sequences are also encompassed by the present invention. Polynucleotides may be single-stranded (coding or antisense) or double-stranded, and may be DNA (genomic, cDNA or synthetic) or RNA molecules. RNA molecules include HnRNA molecules, which contain introns and correspond to a DNA molecule in a one-to-one manner, and mRNA molecules, which do not contain introns. Additional coding or non-coding sequences may, but need not, be present within a polynucleotide of the present invention, and a polynucleotide may, but need not, be linked to other molecules and/or support materials.

Polynucleotides may comprise a native sequence (i.e., an endogenous sequence that encodes a colon tumor protein or a portion thereof) or may comprise a variant of such a sequence. Polynucleotide variants may contain one or more substitutions, additions, deletions and/or insertions such that the immunogenicity of the encoded polypeptide is not diminished, relative to a native tumor protein. The effect on the immunogenicity of the encoded polypeptide may generally be assessed as described herein. Variants preferably exhibit at least about 70% identity, more preferably at least about 80% identity and most preferably at least about 90% identity to a polynucleotide sequence that encodes a native colon tumor protein or a portion thereof.

Two polynucleotide or polypeptide sequences are said to be “identical” if the sequence of nucleotides or amino acids in the two sequences is the same when aligned for maximum correspondence as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J. (1990) Unified Approach to Alignment and Phylogenes pp. 626-645 Methods in Enzymology vol. 183, Academic Press, Inc., San Diego, Calif.; Higgins, D. G. and Sharp, P. M. (1989) CABIOS 5:151-153; Myers, E. W. and Muller W. (1988) CABIOS 4:11-17; Robinson, E. D. (1971) Comb. Theor 11:105; Santou, N. Nes, M. (1987) Mol. Biol. Evol. 4:406-425; Sneath, P. H. A. and Sokal, R. R. (1973) Numerical Taxonomy—the Principles and Practice of Numerical Taxonomy, Freeman Press, San Francisco, Calif.; Wilbur, W. J. and Lipman, D. J. (1983) Proc. Natl. Acad., Sci. USA 80:726-730.

Preferably, the “percentage of sequence identity” is determined by comparing two optimally aligned sequences over a window of comparison of at least 20 positions, wherein the portion of the polynucleotide or polypeptide sequence in the comparison window may comprise additions or deletions (i.e. gaps) of 20 percent or less, usually 5 to 15 percent, or 10 to 12 percent, as compared to the reference sequence (which does not comprise additions or deletions) for optimal alignment of the two sequences. The percentage is calculated by determining the number of positions at which the identical nucleic acid bases or amino acid residue occurs in both sequences to yield the number of matched positions, dividing the number of matched positions by the total number of positions in the reference sequence (i.e. the window size) and multiplying the results by 100 to yield the percentage of sequence identity.

Variants may also, or alternatively, be substantially homologous to a native gene, or a portion or complement thereof. Such polynucleotide variants are capable of hybridizing under moderately stringent conditions to a naturally occurring DNA sequence encoding a native colon tumor protein (or a complementary sequence). Suitable moderately stringent conditions include prewashing in a solution of 5×SSC, 0.5% SDS, 1.0 mM EDTA (pH 8.0); hybridizing at 50° C.-65° C., 5×SSC, overnight; followed by washing twice at 65° C. for 20 minutes with each of 2×, 0.5× and 0.2×SSC containing 0.1% SDS.

It will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the present invention. Further, alleles of the genes comprising the polynucleotide sequences provided herein are within the scope of the present invention. Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

Polynucleotides may be prepared using any of a variety of techniques. For example, a polynucleotide may be identified, as described in more detail below, by screening a microarray of cDNAs for tumor-associated expression (i.e., expression that is at least two fold greater in a colon tumor than in normal tissue, as determined using a representative assay provided herein). Such screens may be performed using a Synteni microarray (Palo Alto, Calif.) according to the manufacturer's instructions (and essentially as described by Schena et al., Proc. Natl. Acad. Sci. USA 93:10614-10619, 1996 and Heller et al., Proc. Natl. Acad. Sci. USA 94:2150-2155, 1997). Alternatively, polypeptides may be amplified from cDNA prepared from cells expressing the proteins described herein, such as colon tumor cells. Such polynucleotides may be amplified via polymerase chain reaction (PCR). For this approach, sequence-specific primers may be designed based on the sequences provided herein, and may be purchased or synthesized.

An amplified portion may be used to isolate a full length gene from a suitable library (e.g., a colon tumor cDNA library) using well known techniques. Within such techniques, a library (cDNA or genomic) is screened using one or more polynucleotide probes or primers suitable for amplification. Preferably, a library is size-selected to include larger molecules. Random primed libraries may also be preferred for identifying 5′ and upstream regions of genes. Genomic libraries are preferred for obtaining introns and extending 5′ sequences.

For hybridization techniques, a partial sequence may be labeled (e.g., by nick-translation or end-labeling with ³²p) using well known techniques. A bacterial or bacteriophage library is then screened by hybridizing filters containing denatured bacterial colonies (or lawns containing phage plaques) with the labeled probe (see Sambrook et al., Molecular Cloning: A Laboratory Manual, Cold Spring Harbor Laboratories, Cold Spring Harbor, N.Y., 1989). Hybridizing colonies or plaques are selected and expanded, and the DNA is isolated for further analysis. cDNA clones may be analyzed to determine the amount of additional sequence by, for example, PCR using a primer from the partial sequence and a primer from the vector. Restriction maps and partial sequences may be generated to identify one or more overlapping clones. The complete sequence may then be determined using standard techniques, which may involve generating a series of deletion clones. The resulting overlapping sequences are then assembled into a single contiguous sequence. A full length cDNA molecule can be generated by ligating suitable fragments, using well known techniques.

Alternatively, there are numerous amplification techniques for obtaining a full length coding sequence from a partial cDNA sequence. Within such techniques, amplification is generally performed via PCR. Any of a variety of commercially available kits may be used to perform the amplification step. Primers may be designed using, for example, software well known in the art. Primers are preferably 22-30 nucleotides in length, have a GC content of at least 50% and anneal to the target sequence at temperatures of about 68° C. to 72° C. The amplified region may be sequenced as described above, and overlapping sequences assembled into a contiguous sequence.

One such amplification technique is inverse PCR (see Triglia et al., Nucl. Acids Res. 16:8186, 1988), which uses restriction enzymes to generate a fragment in the known region of the gene. The fragment is then circularized by intramolecular ligation and used as a template for PCR with divergent primers derived from the known region. Within an alternative approach, sequences adjacent to a partial sequence may be retrieved by amplification with a primer to a linker sequence and a primer specific to a known region. The amplified sequences are typically subjected to a second round of amplification with the same linker primer and a second primer specific to the known region. A variation on this procedure, which employs two primers that initiate extension in opposite directions from the known sequence, is described in WO 96/38591. Another such technique is known as “rapid amplification of cDNA ends” or RACE. This technique involves the use of an internal primer and an external primer, which hybridizes to a polyA region or vector sequence, to identify sequences that are 5′ and 3′ of a known sequence. Additional techniques include capture PCR (Lagerstrom et al., PCR Methods Applic. 1:111-19, 1991) and walking PCR (Parker et al., Nucl. Acids Res. 19:3055-60, 1991). Other methods employing amplification may also be employed to obtain a full length cDNA sequence.

In certain instances, it is possible to obtain a full length cDNA sequence by analysis of sequences provided in an expressed sequence tag (EST) database, such as that available from GenBank. Searches for overlapping ESTs may generally be performed using well known programs (e.g., NCBI BLAST searches), and such ESTs may be used to generate a contiguous full length sequence.

Certain nucleic acid sequences of cDNA molecules encoding portions of colon tumor proteins are provided in SEQ ID NO: 1-121, 123-197, 205-630, 632-684, 686, 690-691, 694-1058, 1069, 1071-1076, 1082, 1084, 1086, 1092, 1094, 1096-1101, 1103-1106, 1111-1114, 1119, and 1120. These polynucleotides were isolated from colon tumor cDNA libraries using conventional and/or PCR-based subtraction techniques, as described below.

Polynucleotide variants may generally be prepared by any method known in the art, including chemical synthesis by, for example, solid phase phosphoramidite chemical synthesis. Modifications in a polynucleotide sequence may also be introduced using standard mutagenesis techniques, such as oligonucleotide-directed site-specific mutagenesis (see Adelman et al., DNA 2:183, 1983). Alternatively, RNA molecules may be generated by in vitro or in vivo transcription of DNA sequences encoding a colon tumor protein, or portion thereof, provided that the DNA is incorporated into a vector with a suitable RNA polymerase promoter (such as T7 or SP6). Certain portions may be used to prepare an encoded polypeptide, as described herein. In addition, or alternatively, a portion may be administered to a patient such that the encoded polypeptide is generated in vivo (e.g., by transfecting antigen-presenting cells, such as dendritic cells, with a cDNA construct encoding a colon tumor polypeptide, and administering the transfected cells to the patient).

A portion of a sequence complementary to a coding sequence (i.e., an antisense polynucleotide) may also be used as a probe or to modulate gene expression. cDNA constructs that can be transcribed into antisense RNA may also be introduced into cells of tissues to facilitate the production of antisense RNA. An antisense polynucleotide may be used, as described herein, to inhibit expression of a tumor protein. Antisense technology can be used to control gene expression through triple-helix formation, which compromises the ability of the double helix to open sufficiently for the binding of polymerases, transcription factors or regulatory molecules (see Gee et al., In Huber and Carr, Molecular and Immunologic Approaches, Futura Publishing Co. (Mt. Kisco, N.Y.; 1994)). Alternatively, an antisense molecule may be designed to hybridize with a control region of a gene (e.g., promoter, enhancer or transcription initiation site), and block transcription of the gene; or to block translation by inhibiting binding of a transcript to ribosomes.

A portion of a coding sequence, or of a complementary sequence, may also be designed as a probe or primer to detect gene expression. Probes may be labeled with a variety of reporter groups, such as radionuclides and enzymes, and are preferably at least 10 nucleotides in length, more preferably at least 20 nucleotides in length and still more preferably at least 30 nucleotides in length. Primers, as noted above, are preferably 22-30 nucleotides in length.

Any polynucleotide may be further modified to increase stability in vivo. Possible modifications include, but are not limited to, the addition of flanking sequences at the 5′ and/or 3′ ends; the use of phosphorothioate or 2′ O-methyl rather than phosphodiesterase linkages in the backbone; and/or the inclusion of nontraditional bases such as inosine, queosine and wybutosine, as well as acetyl- methyl-, thio- and other modified forms of adenine, cytidine, guanine, thymine and uridine.

Nucleotide sequences as described herein may be joined to a variety of other nucleotide sequences using established recombinant DNA techniques. For example, a polynucleotide may be cloned into any of a variety of cloning vectors, including plasmids, phagemids, lambda phage derivatives and cosmids. Vectors of particular interest include expression vectors, replication vectors, probe generation vectors and sequencing vectors. In general, a vector will contain an origin of replication functional in at least one organism, convenient restriction endonuclease sites and one or more selectable markers. Other elements will depend upon the desired use, and will be apparent to those of ordinary skill in the art.

Within certain embodiments, polynucleotides may be formulated so as to permit entry into a cell of a mammal, and expression therein. Such formulations are particularly useful for therapeutic purposes, as described below. Those of ordinary skill in the art will appreciate that there are many ways to achieve expression of a polynucleotide in a target cell, and any suitable method may be employed. For example, a polynucleotide may be incorporated into a viral vector such as, but not limited to, adenovirus, adeno associated virus, retrovirus, or vaccinia or other pox virus (e.g., avian pox virus). Techniques for incorporating DNA into such vectors are well known to those of ordinary skill in the art. A retroviral vector may additionally transfer or incorporate a gene for a selectable marker (to aid in the identification or selection of transduced cells) and/or a targeting moiety, such as a gene that encodes a ligand for a receptor on a specific target cell, to render the vector target specific. Targeting may also be accomplished using an antibody, by methods known to those of ordinary skill in the art.

Other formulations for therapeutic purposes include colloidal dispersion systems, such as macromolecule complexes, nanocapsules, microspheres, beads, and lipid-based systems including oil-in-water emulsions, micelles, mixed micelles, and liposomes. A preferred colloidal system for use as a delivery vehicle in vitro and in vivo is a liposome (i.e., an artificial membrane vesicle). The preparation and use of such systems is well known in the art.

Colon Tumor Polypeptides

Within the context of the present invention, polypeptides may comprise at least an immunogenic portion of a colon tumor protein or a variant thereof, as described herein. As noted above, a “colon tumor protein” is a protein that is expressed by colon tumor cells. Proteins that are colon tumor proteins also react detectably within an immunoassay (such as an ELISA) with antisera from a patient with colon cancer. Polypeptides as described herein may be of any length. Additional sequences derived from the native protein and/or heterologous sequences may be present, and such sequences may (but need not) possess further immunogenic or antigenic properties.

An “immunogenic portion,” as used herein is a portion of a protein that is recognized (i.e., specifically bound) by a B-cell and/or T-cell surface antigen receptor. Such immunogenic portions generally comprise at least 5 amino acid residues, more preferably at least 10, and still more preferably at least 20 amino acid residues of a colon tumor protein or a variant thereof. Certain preferred immunogenic portions include peptides in which an N-terminal leader sequence and/or transmembrane domain have been deleted. Other preferred immunogenic portions may contain a small N- and/or C-terminal deletion (e.g., 1-30 amino acids, preferably 5-15 amino acids), relative to the mature protein.

Immunogenic portions may generally be identified using well known techniques, such as those summarized in Paul, Fundamental Immunology, 3rd ed., 243-247 (Raven Press, 1993) and references cited therein. Such techniques include screening polypeptides for the ability to react with antigen-specific antibodies, antisera and/or T-cell lines or clones. As used herein, antisera and antibodies are “antigen-specific” if they specifically bind to an antigen (i.e., they react with the protein in an ELISA or other immunoassay, and do not react detectably with unrelated proteins). Such antisera and antibodies may be prepared as described herein, and using well known techniques. An immunogenic portion of a native colon tumor protein is a portion that reacts with such antisera and/or T-cells at a level that is not substantially less than the reactivity of the fall length polypeptide (e.g., in an ELISA and/or T-cell reactivity assay). Such immunogenic portions may react within such assays at a level that is similar to or greater than the reactivity of the full length polypeptide. Such screens may generally be performed using methods well known to those of ordinary skill in the art, such as those described in Harlow and Lane, Antibodies: A Laboratory Manual, Cold Spring Harbor Laboratory, 1988. For example, a polypeptide may be immobilized on a solid support and contacted with patient sera to allow binding of antibodies within the sera to the immobilized polypeptide. Unbound sera may then be removed and bound antibodies detected using, for example, ¹²⁵I-labeled Protein A.

As noted above, a composition may comprise a variant of a native colon tumor protein. A polypeptide “variant,” as used herein, is a polypeptide that differs from a native colon tumor protein in one or more substitutions, deletions, additions and/or insertions, such that the immunogenicity of the polypeptide is not substantially diminished. In other words, the ability of a variant to react with antigen-specific antisera may be enhanced or unchanged, relative to the native protein, or may be diminished by less than 50%, and preferably less than 20%, relative to the native protein. Such variants may generally be identified by modifying one of the above polypeptide sequences and evaluating the reactivity of the modified polypeptide with antigen-specific antibodies or antisera as described herein. Preferred variants include those in which one or more portions, such as an N-terminal leader sequence or transmembrane domain, have been removed. Other preferred variants include variants in which a small portion (e.g., 1-30 amino acids, preferably 5-15 amino acids) has been removed from the N- and/or C-terminal of the mature protein.

Polypeptide variants preferably exhibit at least about 70%, more preferably at least about 90% and most preferably at least about 95% identity (determined as described above) to the identified polypeptides.

Preferably, a variant contains conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. Amino acid substitutions may generally be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his. A variant may also, or alternatively, contain non-conservative changes. In a preferred embodiment, variant polypeptides differ from a native sequence by substitution, deletion or addition of five amino acids or fewer. Variants may also (or alternatively) be modified by, for example, the deletion or addition of amino acids that have minimal influence on the immunogenicity, secondary structure and hydropathic nature of the polypeptide.

As noted above, polypeptides may comprise a signal (or leader) sequence at the N-terminal end of the protein which co-translationally or post-translationally directs transfer of the protein. The polypeptide may also be conjugated to a linker or other sequence for ease of synthesis, purification or identification of the polypeptide (e.g., poly-His), or to enhance binding of the polypeptide to a solid support. For example, a polypeptide may be conjugated to an immunoglobulin Fc region.

Polypeptides may be prepared using any of a variety of well known techniques. Recombinant polypeptides encoded by DNA sequences as described above may be readily prepared from the DNA sequences using any of a variety of expression vectors known to those of ordinary skill in the art. Expression may be achieved in any appropriate host cell that has been transformed or transfected with an expression vector containing a DNA molecule that encodes a recombinant polypeptide. Suitable host cells include prokaryotes, yeast and higher eukaryotic cells. Preferably, the host cells employed are E. coli, yeast or a mammalian cell line such as COS or CHO. Supernatants from suitable host/vector systems which secrete recombinant protein or polypeptide into culture media may be first concentrated using a commercially available filter. Following concentration, the concentrate may be applied to a suitable purification matrix such as an affinity matrix or an ion exchange resin. Finally, one or more reverse phase HPLC steps can be employed to further purify a recombinant polypeptide.

Portions and other variants having fewer than about 100 amino acids, and generally fewer than about 50 amino acids, may also be generated by synthetic means, using techniques well known to those of ordinary skill in the art. For example, such polypeptides may be synthesized using any of the commercially available solid-phase techniques, such as the Merrifield solid-phase synthesis method, where amino acids are sequentially added to a growing amino acid chain. See Merrifield, J. Am. Chem. Soc. 85:2149-2146, 1963. Equipment for automated synthesis of polypeptides is commercially available from suppliers such as Perkin Elmer/Applied BioSystems Division (Foster City, Calif.), and may be operated according to the manufacturer's instructions.

Within certain specific embodiments, a polypeptide may be a fusion protein that comprises multiple polypeptides as described herein, or that comprises at least one polypeptide as described herein and an unrelated sequence, such as a known tumor protein. A fusion partner may, for example, assist in providing T helper epitopes (an immunological fusion partner), preferably T helper epitopes recognized by humans, or may assist in expressing the protein (an expression enhancer) at higher yields than the native recombinant protein. Certain preferred fusion partners are both immunological and expression enhancing fusion partners. Other fusion partners may be selected so as to increase the solubility of the protein or to enable the protein to be targeted to desired intracellular compartments. Still further fusion partners include affinity tags, which facilitate purification of the protein.

Fusion proteins may generally be prepared using standard techniques, including chemical conjugation. Preferably, a fusion protein is expressed as a recombinant protein, allowing the production of increased levels, relative to a non-fused protein, in an expression system. Briefly, DNA sequences encoding the polypeptide components may be assembled separately, and ligated into an appropriate expression vector. The 3′ end of the DNA sequence encoding one polypeptide component is ligated, with or without a peptide linker, to the 5′ end of a DNA sequence encoding the second polypeptide component so that the reading frames of the sequences are in phase. This permits translation into a single fusion protein that retains the biological activity of both component polypeptides.

A peptide linker sequence may be employed to separate the first and the second polypeptide components by a distance sufficient to ensure that each polypeptide folds into its secondary and tertiary structures. Such a peptide linker sequence is incorporated into the fusion protein using standard techniques well known in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., Gene 40:39-46, 1985; Murphy et al., Proc. Nati. Acad. Sci. USA 83:8258-8262, 1986; U.S. Pat. No. 4,935,233 and U.S. Pat. No. 4,751,180. The linker sequence may generally be from 1 to about 50 amino acids in length. Linker sequences are not required when the first and second polypeptides have non-essential N-terminal amino acid regions that can be used to separate the functional domains and prevent steric interference.

The ligated DNA sequences are operably linked to suitable transcriptional or translational regulatory elements. The regulatory elements responsible for expression of DNA are located only 5′ to the DNA sequence encoding the first polypeptides. Similarly, stop codons required to end translation and transcription termination signals are only present 3′ to the DNA sequence encoding the second polypeptide.

Fusion proteins are also provided that comprise a polypeptide of the present invention together with an unrelated immunogenic protein. Preferably the immunogenic protein is capable of eliciting a recall response. Examples of such proteins include tetanus, tuberculosis and hepatitis proteins (see, for example, Stoute et al. New Engl. J Med., 336:86-91, 1997).

Within preferred embodiments, an immunological fusion partner is derived from protein D, a surface protein of the gram-negative bacterium Haemophilus influenza B (WO 91/18926). Preferably, a protein D derivative comprises approximately the first third of the protein (e.g., the first N-terminal 100-110 amino acids), and a protein D derivative may be lipidated. Within certain preferred embodiments, the first 109 residues of a Lipoprotein D fusion partner is included on the N-terminus to provide the polypeptide with additional exogenous T-cell epitopes and to increase the expression level in E. coli (thus functioning as an expression enhancer). The lipid tail ensures optimal presentation of the antigen to antigen presenting cells. Other fusion partners include the non-structural protein from influenzae virus, NS1 (hemaglutinin). Typically, the N-terminal 81 amino acids are used, although different fragments that include T-helper epitopes may be used.

In another embodiment, the immunological fusion partner is the protein known as LYTA, or a portion thereof (preferably a C-terminal portion). LYTA is derived from Streptococcus pneumoniae, which synthesizes an N-acetyl-L-alanine amidase known as amidase LYTA (encoded by the LytA gene; Gene 43:265-292, 1986). LYTA is an autolysin that specifically degrades certain bonds in the peptidoglycan backbone. The C-terminal domain of the LYTA protein is responsible for the affinity to the choline or to some choline analogues such as DEAE. This property has been exploited for the development of E. coli C-LYTA expressing plasmids useful for expression of fusion proteins. Purification of hybrid proteins containing the C-LYTA fragment at the amino terminus has been described (see Biotechnology 10:795-798, 1992). Within a preferred embodiment, a repeat portion of LYTA may be incorporated into a fusion protein. A repeat portion is found in the C-terminal region starting at residue 178. A particularly preferred repeat portion incorporates residues 188-305.

In another embodiment, a Mycobacterium tuberculosis-derived Ra12 polynucleotide is linked to at least an immunogenic portion of a polynucleotide of this invention. Ra12 compositions and methods for their use in enhancing expression of heterologous polynucleotide sequences is described in U.S. patent application Ser. No. 60/158,585, the disclosure of which is incorporated herein by reference in its entirety. Briefly, Ra12 refers to a polynucleotide region that is a subsequence of a Mycobacterium tuberculosis MTB32A nucleic acid. MTB32A is a serine protease of 32 KD molecular weight encoded by a gene in virulent and avirulent strains of M. tuberculosis. The nucleotide sequence and amino acid sequence of MTB32A have been described (for example, U.S. patent application Ser. No. 60/158,585; see also, Skeiky et al., Infection and Immun. (1999) 67:3998-4007, incorporated herein by reference). Surprisingly, it was discovered that a 14 KD C-terminal fragment of the MTB32A coding sequence expresses at high levels on its own and remains as a soluble protein throughout the purification process. Moreover, Ra12 may enhance the immunogenicity of heterologous antigenic polypeptides with which it is fused. This 14 KD C-terminal fragment of the MTB32A is referred herein as Ra12 and represents a fragment comprising some or all of amino acid residues 192 to 323 of MTB32A.

Recombinant nucleic acids, which encode a fusion polypeptide comprising a Ra12 polypeptide and a heterologous colon tumor polypeptide of interest, can be readily constructed by conventional genetic engineering techniques. Recombinant nucleic acids are constructed so that, preferably, a Ra12 polynucleotide sequence is located 5′ to a selected heterologous colon tumor polynucleotide sequence. It may also be appropriate to place a Ra12 polynucleotide sequence 3′ to a selected heterologous polynucleotide sequence or to insert a heterologous polynucleotide sequence into a site within a Ra12 polynucleotide sequence.

In addition, any suitable polynucleotide that encodes a Ra12 or a portion or other variant thereof can be used in constructing recombinant fusion polynucleotides comprising Ra12 and one or more colon tumor polynucleotides disclosed herein. Preferred Ra12 polynucleotides generally comprise at least about 15 consecutive nucleotides, at least about 30 nucleotides, at least about 60 nucleotides, at least about 100 nucleotides, at least about 200 nucleotides, or at least about 300 nucleotides that encode a portion of a Ra12 polypeptide.

Ra12 polynucleotides may comprise a native sequence (i.e., an endogenous sequence that encodes a Ra12 polypeptide or a portion thereof) or may comprise a variant of such a sequence. Ra12 polynucleotide variants may contain one or more substitutions, additions, deletions and/or insertions such that the biological activity of the encoded fusion polypeptide is not substantially diminished, relative to a fusion polypeptide comprising a native Ra12 polypeptide. Variants preferably exhibit at least about 70% identity, more preferably at least about 80% identity and most preferably at least about 90% identity to a polynucleotide sequence that encodes a native Ra12 polypeptide or a portion thereof.

In general, polypeptides (including fusion proteins) and polynucleotides as described herein are isolated. An “isolated” polypeptide or polynucleotide is one that is removed from its original environment. For example, a naturally-occurring protein is isolated if it is separated from some or all of the coexisting materials in the natural system. Preferably, such polypeptides are at least about 90% pure, more preferably at least about 95% pure and most preferably at least about 99% pure. A polynucleotide is considered to be isolated if, for example, it is cloned into a vector that is not a part of the natural environment.

Binding Agents

The present invention further provides agents, such as antibodies and antigen-binding fragments thereof, that specifically bind to a colon tumor protein. As used herein, an antibody, or antigen-binding fragment thereof, is said to “specifically bind” to a colon tumor protein if it reacts at a detectable level (within, for example, an ELISA) with a colon tumor protein, and does not react detectably with unrelated proteins under similar conditions. As used herein, “binding” refers to a noncovalent association between two separate molecules such that a complex is formed. The ability to bind may be evaluated by, for example, determining a binding constant for the formation of the complex. The binding constant is the value obtained when the concentration of the complex is divided by the product of the component concentrations. In general, two compounds are said to “bind,” in the context of the present invention, when the binding constant for complex formation exceeds about 10³ L/mol. The binding constant may be determined using methods well known in the art.

Binding agents may be further capable of differentiating between patients with and without a cancer, such as colon cancer, using the representative assays provided herein. In other words, antibodies or other binding agents that bind to a colon tumor protein will generate a signal indicating the presence of a cancer in at least about 20% of patients with the disease, and will generate a negative signal indicating the absence of the disease in at least about 90% of individuals without the cancer. To determine whether a binding agent satisfies this requirement, biological samples (e.g., blood, sera, sputum, urine and/or tumor biopsies) from patients with and without a cancer (as determined using standard clinical tests) may be assayed as described herein for the presence of polypeptides that bind to the binding agent. It will be apparent that a statistically significant number of samples with and without the disease should be assayed. Each binding agent should satisfy the above criteria; however, those of ordinary skill in the art will recognize that binding agents may be used in combination to improve sensitivity.

Any agent that satisfies the above requirements may be a binding agent. For example, a binding agent may be a ribosome, with or without a peptide component, an RNA molecule or a polypeptide. In a preferred embodiment, a binding agent is an antibody or an antigen-binding fragment thereof. Antibodies may be prepared by any of a variety of techniques known to those of ordinary skill in the art. See, e.g., Harlow and Lane, Antibodies: A Laboratory Manual, Cold Spring Harbor Laboratory, 1988. In general, antibodies can be produced by cell culture techniques, including the generation of monoclonal antibodies as described herein, or via transfection of antibody genes into suitable bacterial or mammalian cell hosts, in order to allow for the production of recombinant antibodies. In one technique, an immunogen comprising the polypeptide is initially injected into any of a wide variety of mammals (e.g., mice, rats, rabbits, sheep or goats). In this step, the polypeptides of this invention may serve as the immunogen without modification. Alternatively, particularly for relatively short polypeptides, a superior immune response may be elicited if the polypeptide is joined to a carrier protein, such as bovine serum albumin or keyhole limpet hemocyanin. The immunogen is injected into the animal host, preferably according to a predetermined schedule incorporating one or more booster immunizations, and the animals are bled periodically. Polyclonal antibodies specific for the polypeptide may then be purified from such antisera by, for example, affinity chromatography using the polypeptide coupled to a suitable solid support.

Monoclonal antibodies specific for an antigenic polypeptide of interest may be prepared, for example, using the technique of Kohler and Milstein, Eur. J. Immunol. 6:511-519, 1976, and improvements thereto. Briefly, these methods involve the preparation of immortal cell lines capable of producing antibodies having the desired specificity (i.e., reactivity with the polypeptide of interest). Such cell lines may be produced, for example, from spleen cells obtained from an animal immunized as described above. The spleen cells are then immortalized by, for example, fusion with a myeloma cell fusion partner, preferably one that is syngeneic with the immunized animal. A variety of fusion techniques may be employed. For example, the spleen cells and myeloma cells may be combined with a nonionic detergent for a few minutes and then plated at low density on a selective medium that supports the growth of hybrid cells, but not myeloma cells. A preferred selection technique uses HAT (hypoxanthine, aminopterin, thymidine) selection. After a sufficient time, usually about 1 to 2 weeks, colonies of hybrids are observed. Single colonies are selected and their culture supernatants tested for binding activity against the polypeptide. Hybridomas having high reactivity and specificity are preferred.

Monoclonal antibodies may be isolated from the supernatants of growing hybridoma colonies. In addition, various techniques may be employed to enhance the yield, such as injection of the hybridoma cell line into the peritoneal cavity of a suitable vertebrate host, such as a mouse. Monoclonal antibodies may then be harvested from the ascites fluid or the blood. Contaminants may be removed from the antibodies by conventional techniques, such as chromatography, gel filtration, precipitation, and extraction. The polypeptides of this invention may be used in the purification process in, for example, an affinity chromatography step.

Within certain embodiments, the use of antigen-binding fragments of antibodies may be preferred. Such fragments include Fab fragments, which may be prepared using standard techniques. Briefly, immunoglobulins may be purified from rabbit serum by affinity chromatography on Protein A bead columns (Harlow and Lane, Antibodies: A Laboratory Manual, Cold Spring Harbor Laboratory, 1988) and digested by papain to yield Fab and Fc fragments. The Fab and Fc fragments may be separated by affinity chromatography on protein A bead columns.

Monoclonal antibodies of the present invention may be coupled to one or more therapeutic agents. Suitable agents in this regard include radionuclides, differentiation inducers, drugs, toxins, and derivatives thereof. Preferred radionuclides include ⁹⁰Y, ¹²³I, ¹²⁵I, ¹³¹I, ¹⁸⁶Re, ¹⁸⁸Re, ²¹¹At, and ²¹²Bi. Preferred drugs include methotrexate, and pyrimidine and purine analogs. Preferred differentiation inducers include phorbol esters and butyric acid. Preferred toxins include ricin, abrin, diptheria toxin, cholera toxin, gelonin, Pseudomonas exotoxin, Shigella toxin, and pokeweed antiviral protein.

A therapeutic agent may be coupled (e.g., covalently bonded) to a suitable monoclonal antibody either directly or indirectly (e.g., via a linker group). A direct reaction between an agent and an antibody is possible when each possesses a substituent capable of reacting with the other. For example, a nucleophilic group, such as an amino or sulfhydryl group, on one may be capable of reacting with a carbonyl-containing group, such as an anhydride or an acid halide, or with an alkyl group containing a good leaving group (e.g., a halide) on the other.

Alternatively, it may be desirable to couple a therapeutic agent and an antibody via a linker group. A linker group can function as a spacer to distance an antibody from an agent in order to avoid interference with binding capabilities. A linker group can also serve to increase the chemical reactivity of a substituent on an agent or an antibody, and thus increase the coupling efficiency. An increase in chemical reactivity may also facilitate the use of agents, or functional groups on agents, which otherwise would not be possible.

It will be evident to those skilled in the art that a variety of bifunctional or polyfunctional reagents, both homo- and hetero-functional (such as those described in the catalog of the Pierce Chemical Co., Rockford, Ill.), may be employed as the linker group. Coupling may be effected, for example, through amino groups, carboxyl groups, sulfhydryl groups or oxidized carbohydrate residues. There are numerous references describing such methodology, e.g., U.S. Pat. No. 4,671,958, to Rodwell et al.

Where a therapeutic agent is more potent when free from the antibody portion of the immunoconjugates of the present invention, it may be desirable to use a linker group which is cleavable during or upon internalization into a cell. A number of different cleavable linker groups have been described. The mechanisms for the intracellular release of an agent from these linker groups include cleavage by reduction of a disulfide bond (e.g., U.S. Pat. No. 4,489,710, to Spitler), by irradiation of a photolabile bond (e.g., U.S. Pat. No. 4,625,014, to Senter et al.), by hydrolysis of derivatized amino acid side chains (e.g., U.S. Pat. No. 4,638,045, to Kohn et al.), by serum complement-mediated hydrolysis (e.g., U.S. Pat. No. 4,671,958, to Rodwell et al.), and acid-catalyzed hydrolysis (e.g., U.S. Pat. No. 4,569,789, to Blattler et al.).

It may be desirable to couple more than one agent to an antibody. In one embodiment, multiple molecules of an agent are coupled to one antibody molecule. In another embodiment, more than one type of agent may be coupled to one antibody. Regardless of the particular embodiment, immunoconjugates with more than one agent may be prepared in a variety of ways. For example, more than one agent may be coupled directly to an antibody molecule, or linkers which provide multiple sites for attachment can be used. Alternatively, a carrier can be used.

A carrier may bear the agents in a variety of ways, including covalent bonding either directly or via a linker group. Suitable carriers include proteins such as albumins (e.g., U.S. Pat. No. 4,507,234, to Kato et al.), peptides and polysaccharides such as aminodextran (e.g., U.S. Pat. No. 4,699,784, to Shih et al.). A carrier may also bear an agent by noncovalent bonding or by encapsulation, such as within a liposome vesicle (e.g., U.S. Pat. Nos. 4,429,008 and 4,873,088). Carriers specific for radionuclide agents include radiohalogenated small molecules and chelating compounds. For example, U.S. Pat. No. 4,735,792 discloses representative radiohalogenated small molecules and their synthesis. A radionuclide chelate may be formed from chelating compounds that include those containing nitrogen and sulfur atoms as the donor atoms for binding the metal, or metal oxide, radionuclide. For example, U.S. Pat. No. 4,673,562, to Davison et al. discloses representative chelating compounds and their synthesis.

A variety of routes of administration for the antibodies and immunoconjugates may be used. Typically, administration will be intravenous, intramuscular, subcutaneous or in the bed of a resected tumor. It will be evident that the precise dose of the antibody/immunoconjugate will vary depending upon the antibody used, the antigen density on the tumor, and the rate of clearance of the antibody.

T Cells

Immunotherapeutic compositions may also, or alternatively, comprise T cells specific for a colon tumor protein. Such cells may generally be prepared in vitro or ex vivo, using standard procedures. For example, T cells may be isolated from bone marrow, peripheral blood, or a fraction of bone marrow or peripheral blood of a patient, using a commercially available cell separation system, such as the ISOLEX™ system, available from Nexell Therapeutics Inc., Irvine, Calif. Alternatively, T cells may be derived from related or unrelated humans, non-human mammals, cell lines or cultures.

T cells may be stimulated with a colon tumor polypeptide, polynucleotide encoding a colon tumor polypeptide and/or an antigen presenting cell (APC) that expresses such a polypeptide. Such stimulation is performed under conditions and for a time sufficient to permit the generation of T cells that are specific for the polypeptide. Preferably, a colon tumor polypeptide or polynucleotide is present within a delivery vehicle, such as a microsphere, to facilitate the generation of specific T cells.

T cells are considered to be specific for a colon tumor polypeptide if the T cells kill target cells coated with the polypeptide or expressing a gene encoding the polypeptide. T cell specificity may be evaluated using any of a variety of standard techniques. For example, within a chromium release assay or proliferation assay, a stimulation index of more than two fold increase in lysis and/or proliferation, compared to negative controls, indicates T cell specificity. Such assays may be performed, for example, as described in Chen et al., Cancer Res. 54:1065-1070, 1994. Alternatively, detection of the proliferation of T cells may be accomplished by a variety of known techniques. For example, T cell proliferation can be detected by measuring an increased rate of DNA synthesis (e.g., by pulse-labeling cultures of T cells with tritiated thymidine and measuring the amount of tritiated thymidine incorporated into DNA). Contact with a colon tumor polypeptide (100 ng/ml -100 μg/ml, preferably 200 ng/ml -25 μg/ml) for 3-7 days should result in at least a two fold increase in proliferation of the T cells. Contact as described above for 2-3 hours should result in activation of the T cells, as measured using standard cytokine assays in which a two fold increase in the level of cytokine release (e.g., TNF or IFN-γ) is indicative of T cell activation (see Coligan et al., Current Protocols in Immunology, vol. 1, Wiley Interscience (Greene 1998)). T cells that have been activated in response to a colon tumor polypeptide, polynucleotide or polypeptide-expressing APC may be CD4⁺ and/or CD8⁺. Colon tumor protein-specific T cells may be expanded using standard techniques. Within preferred embodiments, the T cells are derived from either a patient or a related, or unrelated, donor and are administered to the patient following stimulation and expansion.

For therapeutic purposes, CD4⁺ or CD8⁺ T cells that proliferate in response to a colon tumor polypeptide, polynucleotide or APC can be expanded in number either in vitro or in vivo. Proliferation of such T cells in vitro may be accomplished in a variety of ways. For example, the T cells can be re-exposed to a colon tumor polypeptide, or a short peptide corresponding to an immunogenic portion of such a polypeptide, with or without the addition of T cell growth factors, such as interleukin-2, and/or stimulator cells that synthesize a colon tumor polypeptide. Alternatively, one or more T cells that proliferate in the presence of a colon tumor protein can be expanded in number by cloning. Methods for cloning cells are well known in the art, and include limiting dilution.

Pharmaceutical Compositions and Vaccines

Within certain aspects, polypeptides, polynucleotides, T cells and/or binding agents disclosed herein may be incorporated into pharmaceutical compositions or immunogenic compositions (i.e., vaccines). Pharmaceutical compositions comprise one or more such compounds and a physiologically acceptable carrier. Vaccines may comprise one or more such compounds and an immunostimulant. An immunostimulant may be any substance that enhances or potentiates an immune response to an exogenous antigen. Examples of immunostimulants include adjuvants, biodegradable microspheres (e.g., polylactic galactide) and liposomes (into which the compound is incorporated; see e.g., Fullerton, U.S. Pat. No. 4,235,877). Vaccine preparation is generally described in, for example, M. F. Powell and M. J. Newman, eds., “Vaccine Design (the subunit and adjuvant approach),” Plenum Press (NY, 1995). Pharmaceutical compositions and vaccines within the scope of the present invention may also contain other compounds, which may be biologically active or inactive. For example, one or more immunogenic portions of other tumor antigens may be present, either incorporated into a fusion polypeptide or as a separate compound, within the composition or vaccine.

A pharmaceutical composition or vaccine may contain DNA encoding one or more of the polypeptides as described above, such that the polypeptide is generated in situ. As noted above, the DNA may be present within any of a variety of delivery systems known to those of ordinary skill in the art, including nucleic acid expression systems, bacteria and viral expression systems. Numerous gene delivery techniques are well known in the art, such as those described by Rolland, Crit. Rev. Therap. Drug Carrier Systems 15:143-198, 1998, and references cited therein. Appropriate nucleic acid expression systems contain the necessary DNA sequences for expression in the patient (such as a suitable promoter and terminating signal). Bacterial delivery systems involve the administration of a bacterium (such as Bacillus-Calmette-Guerrin) that expresses an immunogenic portion of the polypeptide on its cell surface or secretes such an epitope. In a preferred embodiment, the DNA may be introduced using a viral expression system (e.g., vaccinia or other pox virus, retrovirus, or adenovirus), which may involve the use of a non-pathogenic (defective), replication competent virus. Suitable systems are disclosed, for example, in Fisher-Hoch et al., Proc. Natl. Acad. Sci. USA 86:317-321, 1989; Flexner et al., Ann. N.Y. Acad. Sci. 569:86-103, 1989; Flexner et al., Vaccine 8:17-21, 1990; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner, Biotechniques 6:616-627, 1988; Rosenfeld et al., Science 252:431-434, 1991; Kolls et al., Proc. Natl. Acad. Sci. USA 91:215-219, 1994; Kass-Eisler et al., Proc. Natl. Acad. Sci. USA 90:11498-11502, 1993; Guzman et al., Circulation 88:2838-2848, 1993; and Guzman et al., Cir. Res. 73:1202-1207, 1993. Techniques for incorporating DNA into such expression systems are well known to those of ordinary skill in the art. The DNA may also be “naked,” as described, for example, in Ulmer et al., Science 259:1745-1749, 1993 and reviewed by Cohen, Science 259:1691-1692, 1993. The uptake of naked DNA may be increased by coating the DNA onto biodegradable beads, which are efficiently transported into the cells.

While any suitable carrier known to those of ordinary skill in the art may be employed in the pharmaceutical compositions of this invention, the type of carrier will vary depending on the mode of administration. Compositions of the present invention may be formulated for any appropriate manner of administration, including for example, topical, oral, nasal, intravenous, intracranial, intraperitoneal, subcutaneous or intramuscular administration. For parenteral administration, such as subcutaneous injection, the carrier preferably comprises water, saline, alcohol, a fat, a wax or a buffer. For oral administration, any of the above carriers or a solid carrier, such as mannitol, lactose, starch, magnesium stearate, sodium saccharine, talcum, cellulose, glucose, sucrose, and magnesium carbonate, may be employed. Biodegradable microspheres (e.g., polylactate polyglycolate) may also be employed as carriers for the pharmaceutical compositions of this invention. Suitable biodegradable microspheres are disclosed, for example, in U.S. Pat. Nos. 4,897,268 and 5,075,109.

Such compositions may also comprise buffers (e.g., neutral buffered saline or phosphate buffered saline), carbohydrates (e.g., glucose, mannose, sucrose or dextrans), mannitol, proteins, polypeptides or amino acids such as glycine, antioxidants, chelating agents such as EDTA or glutathione, adjuvants (e.g., aluminum hydroxide) and/or preservatives. Alternatively, compositions of the present invention may be formulated as a lyophilizate. Compounds may also be encapsulated within liposomes using well known technology.

Any of a variety of immunostimulants may be employed in the vaccines of this invention. For example, an adjuvant may be included. Most adjuvants contain a substance designed to protect the antigen from rapid catabolism, such as aluminum hydroxide or mineral oil, and a stimulator of immune responses, such as lipid A, Bortadella pertussis or Mycobacterium tuberculosis derived proteins. Suitable adjuvants are commercially available as, for example, Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham, Philadelphia, Pa.); aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF or interleukin-2, -7, or -12, may also be used as adjuvants.

Within the vaccines provided herein, the adjuvant composition is preferably designed to induce an immune response predominantly of the Th1 type. High levels of Th1-type cytokines (e.g., IFN-γ, TNFα, IL-2 and IL-12) tend to favor the induction of cell mediated immune responses to an administered antigen. In contrast, high levels of Th2-type cytokines (e.g., IL-4, IL-5, IL-6 and IL-10) tend to favor the induction of humoral immune responses. Following application of a vaccine as provided herein, a patient will support an immune response that includes Th1- and Th2-type responses. Within a preferred embodiment, in which a response is predominantly Th1-type, the level of Th1-type cytokines will increase to a greater extent than the level of Th2-type cytokines. The levels of these cytokines may be readily assessed using standard assays. For a review of the families of cytokines, see Mosmann and Coffman, Ann. Rev. Immunol. 7:145-173, 1989.

Preferred adjuvants for use in eliciting a predominantly Th1-type response include, for example, a combination of monophosphoryl lipid A, preferably 3-de-O-acylated monophosphoryl lipid A (3D-MPL), together with an aluminum salt. MPL adjuvants are available from Corixa Corp. (Seattle, Wash.) (see U.S. Pat. Nos. 4,436,727; 4,877,611; 4,866,034 and 4,912,094). CpG-containing oligonucleotides (in which the CpG dinucleotide is unmethylated) also induce a predominantly Th1response. Such oligonucleotides are well known and are described, for example, in WO 96/02555 and WO 99/33488. Immunostimulatory DNA sequences are also described, for example, by Sato et al., Science 273:352, 1996. Another preferred adjuvant is a saponin, preferably QS21 (Aquila Biopharmaceuticals Inc., Framingham, Mass.), which may be used alone or in combination with other adjuvants. For example, an enhanced system involves the combination of a monophosphoryl lipid A and saponin derivative, such as the combination of QS21 and 3D-MPL as described in WO 94/00153, or a less reactogenic composition where the QS21 is quenched with cholesterol, as described in WO 96/33739. Other preferred formulations comprises an oil-in-water emulsion and tocopherol. A particularly potent adjuvant formulation involving QS21, 3D-MPL and tocopherol in an oil-in-water emulsion is described in WO 95/17210.

Other preferred adjuvants include Montanide ISA 720 (Seppic, France), SAF (Chiron, Calif., United States), ISCOMS (CSL), MF-59 (Chiron), the SBAS series of adjuvants (e.g., SBAS-2 or SBAS-4, available from SmithKline Beecham, Rixensart, Belgium), Detox (Ribi ImmunoChem Research Inc., Hamilton, Mont.), RC-529 (Corixa, Seattle, Wash.) and Aminoalkyl glucosaminide 4-phosphates (AGPs).

Any vaccine provided herein may be prepared using well known methods that result in a combination of antigen, immune response enhancer and a suitable carrier or excipient. The compositions described herein may be administered as part of a sustained release formulation (i.e., a formulation such as a capsule, sponge or gel (composed of polysaccharides, for example) that effects a slow release of compound following administration). Such formulations may generally be prepared using well known technology (see, e.g. Coombes et al., Vaccine 14:1429-1438, 1996) and administered by, for example, oral, rectal or subcutaneous implantation, or by implantation at the desired target site. Sustained-release formulations may contain a polypeptide, polynucleotide or antibody dispersed in a carrier matrix and/or contained within a reservoir surrounded by a rate controlling membrane.

Carriers for use within such formulations are biocompatible, and may also be biodegradable; preferably the formulation provides a relatively constant level of active component release. Such carriers include microparticles of poly(lactide-co-glycolide), as well as polyacrylate, latex, starch, cellulose and dextran. Other delayed-release carriers include supramolecular biovectors, which comprise a non-liquid hydrophilic core (e.g., a cross-linked polysaccharide or oligosaccharide) and, optionally, an external layer comprising an amphiphilic compound, such as a phospholipid (see e.g., U.S. Pat. No. 5,151,254 and PCT applications WO 94/20078, WO/94/23701 and WO 96/06638). The amount of active compound contained within a sustained release formulation depends upon the site of implantation, the rate and expected duration of release and the nature of the condition to be treated or prevented.

Any of a variety of delivery vehicles may be employed within pharmaceutical compositions and vaccines to facilitate production of an antigen-specific immune response that targets tumor cells. Delivery vehicles include antigen presenting cells (APCs), such as dendritic cells, macrophages, B cells, monocytes and other cells that may be engineered to be efficient APCs. Such cells may, but need not, be genetically modified to increase the capacity for presenting the antigen, to improve activation and/or maintenance of the T cell response, to have anti-tumor effects per se and/or to be immunologically compatible with the receiver (i.e., matched HLA haplotype). APCs may generally be isolated from any of a variety of biological fluids and organs, including tumor and peritumoral tissues, and may be autologous, allogeneic, syngeneic or xenogeneic cells.

Certain preferred embodiments of the present invention use dendritic cells or progenitors thereof as antigen-presenting cells. Dendritic cells are highly potent APCs (Banchereau and Steinman, Nature 392:245-251, 1998) and have been shown to be effective as a physiological adjuvant for eliciting prophylactic or therapeutic antitumor immunity (see Timmerman and Levy, Ann. Rev. Med. 50:507-529, 1999). In general, dendritic cells may be identified based on their typical shape (stellate in situ, with marked cytoplasmic processes (dendrites) visible in vitro), their ability to take up, process and present antigens with high efficiency, and their ability to activate naive T cell responses. Dendritic cells may, of course, be engineered to express specific cell-surface receptors or ligands that are not commonly found on dendritic cells in vivo or ex vivo, and such modified dendritic cells are contemplated by the present invention. As an alternative to dendritic cells, secreted vesicles antigen-loaded dendritic cells (called exosomes) may be used within a vaccine (see Zitvogel et al., Nature Med. 4:594-600, 1998).

Dendritic cells and progenitors may be obtained from peripheral blood, bone marrow, tumor-infiltrating cells, peritumoral tissues-infiltrating cells, lymph nodes, spleen, skin, umbilical cord blood or any other suitable tissue or fluid. For example, dendritic cells may be differentiated ex vivo by adding a combination of cytokines such as GM-CSF, IL-4, IL-13 and/or TNFα to cultures of monocytes harvested from peripheral blood. Alternatively, CD34 positive cells harvested from peripheral blood, umbilical cord blood or bone marrow may be differentiated into dendritic cells by adding to the culture medium combinations of GM-CSF, IL-3, TNFα, CD40 ligand, LPS, flt3 ligand and/or other compound(s) that induce differentiation, maturation and proliferation of dendritic cells.

Dendritic cells are conveniently categorized as “immature” and “mature” cells, which allows a simple way to discriminate between two well characterized phenotypes. However, this nomenclature should not be construed to exclude all possible intermediate stages of differentiation. Immature dendritic cells are characterized as APC with a high capacity for antigen uptake and processing, which correlates with the high expression of Fcγ receptor and mannose receptor. The mature phenotype is typically characterized by a lower expression of these markers, but a high expression of cell surface molecules responsible for T cell activation such as class I and class II MHC, adhesion molecules (e.g., CD54 and CD11) and costimulatory molecules (e.g., CD40, CD80, CD86 and 4-1BB).

APCs may generally be transfected with a polynucleotide encoding a colon tumor protein (or portion or other variant thereof) such that the colon tumor polypeptide, or an immunogenic portion thereof, is expressed on the cell surface. Such transfection may take place ex vivo, and a composition or vaccine comprising such transfected cells may then be used for therapeutic purposes, as described herein. Alternatively, a gene delivery vehicle that targets a dendritic or other antigen presenting cell may be administered to a patient, resulting in transfection that occurs in vivo. In vivo and ex vivo transfection of dendritic cells, for example, may generally be performed using any methods known in the art, such as those described in WO 97/24447, or the gene gun approach described by Mahvi et al., Immunology and cell Biology 75:456-460, 1997. Antigen loading of dendritic cells may be achieved by incubating dendritic cells or progenitor cells with the colon tumor polypeptide, DNA (naked or within a plasmid vector) or RNA; or with antigen-expressing recombinant bacterium or viruses (e.g., vaccinia, fowlpox, adenovirus or lentivirus vectors). Prior to loading, the polypeptide may be covalently conjugated to an immunological partner that provides T cell help (e.g., a carrier molecule). Alternatively, a dendritic cell may be pulsed with a non-conjugated immunological partner, separately or in the presence of the polypeptide.

Vaccines and pharmaceutical compositions may be presented in unit-dose or multi-dose containers, such as sealed ampoules or vials. Such containers are preferably hermetically sealed to preserve sterility of the formulation until use. In general, formulations may be stored as suspensions, solutions or emulsions in oily or aqueous vehicles. Alternatively, a vaccine or pharmaceutical composition may be stored in a freeze-dried condition requiring only the addition of a sterile liquid carrier immediately prior to use.

Cancer Therapy

In further aspects of the present invention, the compositions described herein may be used for immunotherapy of cancer, such as colon cancer. Within such methods, pharmaceutical compositions and vaccines are typically administered to a patient. As used herein, a “patient” refers to any warm-blooded animal, preferably a human. A patient may or may not be afflicted with cancer. Accordingly, the above pharmaceutical compositions and vaccines may be used to prevent the development of a cancer or to treat a patient afflicted with a cancer. A cancer may be diagnosed using criteria generally accepted in the art, including the presence of a malignant tumor. Pharmaceutical compositions and vaccines may be administered either prior to or following surgical removal of primary tumors and/or treatment such as administration of radiotherapy or conventional chemotherapeutic drugs.

Within certain embodiments, immunotherapy may be active immunotherapy, in which treatment relies on the in vivo stimulation of the endogenous host immune system to react against tumors with the administration of immune response-modifying agents (such as polypeptides and polynucleotides disclosed herein).

Within other embodiments, immunotherapy may be passive immunotherapy, in which treatment involves the delivery of agents with established tumor-immune reactivity (such as effector cells or antibodies) that can directly or indirectly mediate antitumor effects and does not necessarily depend on an intact host immune system. Examples of effector cells include T cells as discussed above, T lymphocytes (such as CD8⁺ cytotoxic T lymphocytes and CD4⁺ T-helper tumor-infiltrating lymphocytes), killer cells (such as Natural Killer cells and lymphokine-activated killer cells), B cells and antigen-presenting cells (such as dendritic cells and macrophages) expressing a polypeptide provided herein. T cell receptors and antibody receptors specific for the polypeptides recited herein may be cloned, expressed and transferred into other vectors or effector cells for adoptive immunotherapy. The polypeptides provided herein may also be used to generate antibodies or anti-idiotypic antibodies (as described above and in U.S. Pat. No. 4,918,164) for passive immunotherapy.

Effector cells may generally be obtained in sufficient quantities for adoptive immunotherapy by growth in vitro, as described herein. Culture conditions for expanding single antigen-specific effector cells to several billion in number with retention of antigen recognition in vivo are well known in the art. Such in vitro culture conditions typically use intermittent stimulation with antigen, often in the presence of cytokines (such as IL-2) and non-dividing feeder cells. As noted above, immunoreactive polypeptides as provided herein may be used to rapidly expand antigen-specific T cell cultures in order to generate a sufficient number of cells for immunotherapy. In particular, antigen-presenting cells, such as dendritic, macrophage, monocyte, fibroblast and/or B cells, may be pulsed with immunoreactive polypeptides or transfected with one or more polynucleotides using standard techniques well known in the art. For example, antigen-presenting cells can be transfected with a polynucleotide having a promoter appropriate for increasing expression in a recombinant virus or other expression system. Cultured effector cells for use in therapy must be able to grow and distribute widely, and to survive long term in vivo. Studies have shown that cultured effector cells can be induced to grow in vivo and to survive long term in substantial numbers by repeated stimulation with antigen supplemented with IL-2 (see, for example, Cheever et al., Immunological Reviews 157:177, 1997).

Alternatively, a vector expressing a polypeptide recited herein may be introduced into antigen presenting cells taken from a patient and clonally propagated ex vivo for transplant back into the same patient. Transfected cells may be reintroduced into the patient using any means known in the art, preferably in sterile form by intravenous, intracavitary, intraperitoneal or intratumor administration.

Routes and frequency of administration of the therapeutic compositions disclosed herein, as well as dosage, will vary from individual to individual, and may be readily established using standard techniques. In general, the pharmaceutical compositions and vaccines may be administered by injection (e.g., intracutaneous, intramuscular, intravenous or subcutaneous), intranasally (e.g., by aspiration) or orally. Preferably, between 1 and 10 doses may be administered over a 52 week period. Preferably, 6 doses are administered, at intervals of 1 month, and booster vaccinations may be given periodically thereafter. Alternate protocols may be appropriate for individual patients. A suitable dose is an amount of a compound that, when administered as described above, is capable of promoting an anti-tumor immune response, and is at least 10-50% above the basal (i.e., untreated) level. Such response can be monitored by measuring the anti-tumor antibodies in a patient or by vaccine-dependent generation of cytolytic effector cells capable of killing the patient's tumor cells in vitro. Such vaccines should also be capable of causing an immune response that leads to an improved clinical outcome (e.g., more frequent remissions, complete or partial or longer disease-free survival) in vaccinated patients as compared to non-vaccinated patients. In general, for pharmaceutical compositions and vaccines comprising one or more polypeptides, the amount of each polypeptide present in a dose ranges from about 25 μg to 5 mg per kg of host. Suitable dose sizes will vary with the size of the patient, but will typically range from about 0.1 mL to about 5 mL.

In general, an appropriate dosage and treatment regimen provides the active compound(s) in an amount sufficient to provide therapeutic and/or prophylactic benefit. Such a response can be monitored by establishing an improved clinical outcome (e.g., more frequent remissions, complete or partial, or longer disease-free survival) in treated patients as compared to non-treated patients. Increases in preexisting immune responses to a colon tumor protein generally correlate with an improved clinical outcome. Such immune responses may generally be evaluated using standard proliferation, cytotoxicity or cytokine assays, which may be performed using samples obtained from a patient before and after treatment.

Methods for Detecting Cancer

In general, a cancer may be detected in a patient based on the presence of one or more colon tumor proteins and/or polynucleotides encoding such proteins in a biological sample (for example, blood, sera, sputum, urine and/or tumor biopsies) obtained from the patient. In other words, such proteins may be used as markers to indicate the presence or absence of a cancer such as colon cancer. In addition, such proteins may be useful for the detection of other cancers. The binding agents provided herein generally permit detection of the level of antigen that binds to the agent in the biological sample. Polynucleotide primers and probes may be used to detect the level of mRNA encoding a tumor protein, which is also indicative of the presence or absence of a cancer. In general, a colon tumor sequence should be present at a level that is at least three fold higher in tumor tissue than in normal tissue

There are a variety of assay formats known to those of ordinary skill in the art for using a binding agent to detect polypeptide markers in a sample. See, e.g., Harlow and Lane, Antibodies: A Laboratory Manual, Cold Spring Harbor Laboratory, 1988. In general, the presence or absence of a cancer in a patient may be determined by (a) contacting a biological sample obtained from a patient with a binding agent; (b) detecting in the sample a level of polypeptide that binds to the binding agent; and (c) comparing the level of polypeptide with a predetermined cut-off value.

In a preferred embodiment, the assay involves the use of binding agent immobilized on a solid support to bind to and remove the polypeptide from the remainder of the sample. The bound polypeptide may then be detected using a detection reagent that contains a reporter group and specifically binds to the binding agent/polypeptide complex. Such detection reagents may comprise, for example, a binding agent that specifically binds to the polypeptide or an antibody or other agent that specifically binds to the binding agent, such as an anti-immunoglobulin, protein G, protein A or a lectin. Alternatively, a competitive assay may be utilized, in which a polypeptide is labeled with a reporter group and allowed to bind to the immobilized binding agent after incubation of the binding agent with the sample. The extent to which components of the sample inhibit the binding of the labeled polypeptide to the binding agent is indicative of the reactivity of the sample with the immobilized binding agent. Suitable polypeptides for use within such assays include full length colon tumor proteins and portions thereof to which the binding agent binds, as described above.

The solid support may be any material known to those of ordinary skill in the art to which the tumor protein may be attached. For example, the solid support may be a test well in a microtiter plate or a nitrocellulose or other suitable membrane. Alternatively, the support may be a bead or disc, such as glass, fiberglass, latex or a plastic material such as polystyrene or polyvinylchloride. The support may also be a magnetic particle or a fiber optic sensor, such as those disclosed, for example, in U.S. Pat. No. 5,359,681. The binding agent may be immobilized on the solid support using a variety of techniques known to those of skill in the art, which are amply described in the patent and scientific literature. In the context of the present invention, the term “immobilization” refers to both noncovalent association, such as adsorption, and covalent attachment (which may be a direct linkage between the agent and functional groups on the support or may be a linkage by way of a cross-linking agent). Immobilization by adsorption to a well in a microtiter plate or to a membrane is preferred. In such cases, adsorption may be achieved by contacting the binding agent, in a suitable buffer, with the solid support for a suitable amount of time. The contact time varies with temperature, but is typically between about 1 hour and about 1 day. In general, contacting a well of a plastic microtiter plate (such as polystyrene or polyvinylchloride) with an amount of binding agent ranging from about 10 ng to about 10 μg, and preferably about 100 ng to about 1 μg, is sufficient to immobilize an adequate amount of binding agent.

Covalent attachment of binding agent to a solid support may generally be achieved by first reacting the support with a bifunctional reagent that will react with both the support and a functional group, such as a hydroxyl or amino group, on the binding agent. For example, the binding agent may be covalently attached to supports having an appropriate polymer coating using benzoquinone or by condensation of an aldehyde group on the support with an amine and an active hydrogen on the binding partner (see, e.g., Pierce Immunotechnology Catalog and Handbook, 1991, at A12-A13).

In certain embodiments, the assay is a two-antibody sandwich assay. This assay may be performed by first contacting an antibody that has been immobilized on a solid support, commonly the well of a microtiter plate, with the sample, such that polypeptides within the sample are allowed to bind to the immobilized antibody. Unbound sample is then removed from the immobilized polypeptide-antibody complexes and a detection reagent (preferably a second antibody capable of binding to a different site on the polypeptide) containing a reporter group is added. The amount of detection reagent that remains bound to the solid support is then determined using a method appropriate for the specific reporter group.

More specifically, once the antibody is immobilized on the support as described above, the remaining protein binding sites on the support are typically blocked. Any suitable blocking agent known to those of ordinary skill in the art, such as bovine serum albumin or Tween 20™ (Sigma Chemical Co., St. Louis, Mo.). The immobilized antibody is then incubated with the sample, and polypeptide is allowed to bind to the antibody. The sample may be diluted with a suitable diluent, such as phosphate-buffered saline (PBS) prior to incubation. In general, an appropriate contact time (i.e., incubation time) is a period of time that is sufficient to detect the presence of polypeptide within a sample obtained from an individual with colon cancer. Preferably, the contact time is sufficient to achieve a level of binding that is at least about 95% of that achieved at equilibrium between bound and unbound polypeptide. Those of ordinary skill in the art will recognize that the time necessary to achieve equilibrium may be readily determined by assaying the level of binding that occurs over a period of time. At room temperature, an incubation time of about 30 minutes is generally sufficient.

Unbound sample may then be removed by washing the solid support with an appropriate buffer, such as PBS containing 0.1% Tween 20™. The second antibody, which contains a reporter group, may then be added to the solid support. Preferred reporter groups include those groups recited above.

The detection reagent is then incubated with the immobilized antibody-polypeptide complex for an amount of time sufficient to detect the bound polypeptide. An appropriate amount of time may generally be determined by assaying the level of binding that occurs over a period of time. Unbound detection reagent is then removed and bound detection reagent is detected using the reporter group. The method employed for detecting the reporter group depends upon the nature of the reporter group. For radioactive groups, scintillation counting or autoradiographic methods are generally appropriate. Spectroscopic methods may be used to detect dyes, luminescent groups and fluorescent groups. Biotin may be detected using avidin, coupled to a different reporter group (commonly a radioactive or fluorescent group or an enzyme). Enzyme reporter groups may generally be detected by the addition of substrate (generally for a specific period of time), followed by spectroscopic or other analysis of the reaction products.

To determine the presence or absence of a cancer, such as colon cancer, the signal detected from the reporter group that remains bound to the solid support is generally compared to a signal that corresponds to a predetermined cut-off value. In one preferred embodiment, the cut-off value for the detection of a cancer is the average mean signal obtained when the immobilized antibody is incubated with samples from patients without the cancer. In general, a sample generating a signal that is three standard deviations above the predetermined cut-off value is considered positive for the cancer. In an alternate preferred embodiment, the cut-off value is determined using a Receiver Operator Curve, according to the method of Sackett et al., Clinical Epidemiology: A Basic Science for Clinical Medicine, Little Brown and Co., 1985, p. 106-7. Briefly, in this embodiment, the cut-off value may be determined from a plot of pairs of true positive rates (i.e., sensitivity) and false positive rates (100%-specificity) that correspond to each possible cut-off value for the diagnostic test result. The cut-off value on the plot that is the closest to the upper left-hand corner (i.e., the value that encloses the largest area) is the most accurate cut-off value, and a sample generating a signal that is higher than the cut-off value determined by this method may be considered positive. Alternatively, the cut-off value may be shifted to the left along the plot, to minimize the false positive rate, or to the right, to minimize the false negative rate. In general, a sample generating a signal that is higher than the cut-off value determined by this method is considered positive for a cancer.

In a related embodiment, the assay is performed in a flow-through or strip test format, wherein the binding agent is immobilized on a membrane, such as nitrocellulose. In the flow-through test, polypeptides within the sample bind to the immobilized binding agent as the sample passes through the membrane. A second, labeled binding agent then binds to the binding agent-polypeptide complex as a solution containing the second binding agent flows through the membrane. The detection of bound second binding agent may then be performed as described above. In the strip test format, one end of the membrane to which binding agent is bound is immersed in a solution containing the sample. The sample migrates along the membrane through a region containing second binding agent and to the area of immobilized binding agent. Concentration of second binding agent at the area of immobilized antibody indicates the presence of a cancer. Typically, the concentration of second binding agent at that site generates a pattern, such as a line, that can be read visually. The absence of such a pattern indicates a negative result. In general, the amount of binding agent immobilized on the membrane is selected to generate a visually discernible pattern when the biological sample contains a level of polypeptide that would be sufficient to generate a positive signal in the two-antibody sandwich assay, in the format discussed above. Preferred binding agents for use in such assays are antibodies and antigen-binding fragments thereof. Preferably, the amount of antibody immobilized on the membrane ranges from about 25 ng to about 1 μg, and more preferably from about 50 ng to about 500 ng. Such tests can typically be performed with a very small amount of biological sample.

Of course, numerous other assay protocols exist that are suitable for use with the tumor proteins or binding agents of the present invention. The above descriptions are intended to be exemplary only. For example, it will be apparent to those of ordinary skill in the art that the above protocols may be readily modified to use colon tumor polypeptides to detect antibodies that bind to such polypeptides in a biological sample. The detection of such colon tumor protein specific antibodies may correlate with the presence of a cancer.

A cancer may also, or alternatively, be detected based on the presence of T cells that specifically react with a colon tumor protein in a biological sample. Within certain methods, a biological sample comprising CD4⁺ and/or CD8⁺ T cells isolated from a patient is incubated with a colon tumor polypeptide, a polynucleotide encoding such a polypeptide and/or an APC that expresses at least an immunogenic portion of such a polypeptide, and the presence or absence of specific activation of the T cells is detected. Suitable biological samples include, but are not limited to, isolated T cells. For example, T cells may be isolated from a patient by routine techniques (such as by Ficoll/Hypaque density gradient centrifugation of peripheral blood lymphocytes). T cells may be incubated in vitro for 2-9 days (typically 4 days) at 37° C. with one or more representative polypeptides (e.g., 5-25 μg/ml). It may be desirable to incubate another aliquot of a T cell sample in the absence of colon tumor polypeptide to serve as a control. For CD4⁺ T cells, activation is preferably detected by evaluating proliferation of the T cells. For CD8⁺ T cells, activation is preferably detected by evaluating cytolytic activity. A level of proliferation that is at least two fold greater and/or a level of cytolytic activity that is at least 20% greater than in disease-free patients indicates the presence of a cancer in the patient.

As noted above, a cancer may also, or alternatively, be detected based on the level of mRNA encoding a colon tumor protein in a biological sample. For example, at least two oligonucleotide primers may be employed in a polymerase chain reaction (PCR) based assay to amplify a portion of a colon tumor cDNA derived from a biological sample, wherein at least one of the oligonucleotide primers is specific for (i.e., hybridizes to) a polynucleotide encoding the colon tumor protein. The amplified cDNA is then separated and detected using techniques well known in the art, such as gel electrophoresis. Similarly, oligonucleotide probes that specifically hybridize to a polynucleotide encoding a colon tumor protein may be used in a hybridization assay to detect the presence of polynucleotide encoding the tumor protein in a biological sample.

To permit hybridization under assay conditions, oligonucleotide primers and probes should comprise an oligonucleotide sequence that has at least about 60%, preferably at least about 75% and more preferably at least about 90%, identity to a portion of a polynucleotide encoding a colon tumor protein that is at least 10 nucleotides, and preferably at least 20 nucleotides, in length. Preferably, oligonucleotide primers and/or probes will hybridize to a polynucleotide encoding a polypeptide disclosed herein under moderately stringent conditions, as defined above. Oligonucleotide primers and/or probes which may be usefully employed in the diagnostic methods described herein preferably are at least 10-40 nucleotides in length. In a preferred embodiment, the oligonucleotide primers comprise at least 10 contiguous nucleotides, more preferably at least 15 contiguous nucleotides, of a DNA molecule having a sequence recited in SEQ ID NO: 1-121, 123-197, 205-630, 632-684, 686, 690-691, 694-1058, 1069, 1071-1076, 1082, 1084, 1086, 1092, 1094, 1096-1101, 1103-1106, 1111-1114, 1119, and 1120. Techniques for both PCR based assays and hybridization assays are well known in the art (see, for example, Mullis et al., Cold Spring Harbor Symp. Quant. Biol., 51:263, 1987; Erlich ed., PCR Technology, Stockton Press, NY, 1989).

One preferred assay employs RT-PCR, in which PCR is applied in conjunction with reverse transcription. Typically, RNA is extracted from a biological sample, such as biopsy tissue, and is reverse transcribed to produce cDNA molecules. PCR amplification using at least one specific primer generates a cDNA molecule, which may be separated and visualized using, for example, gel electrophoresis. Amplification may be performed on biological samples taken from a test patient and from an individual who is not afflicted with a cancer. The amplification reaction may be performed on several dilutions of cDNA spanning two orders of magnitude. A two-fold or greater increase in expression in several dilutions of the test patient sample as compared to the same dilutions of the non-cancerous sample is typically considered positive.

In another embodiment, the disclosed compositions may be used as markers for the progression of cancer. In this embodiment, assays as described above for the diagnosis of a cancer may be performed over time, and the change in the level of reactive polypeptide(s) or polynucleotide evaluated. For example, the assays may be performed every 24-72 hours for a period of 6 months to 1 year, and thereafter performed as needed. In general, a cancer is progressing in those patients in whom the level of polypeptide or polynucleotide detected increases over time. In contrast, the cancer is not progressing when the level of reactive polypeptide or polynucleotide either remains constant or decreases with time.

Certain in vivo diagnostic assays may be performed directly on a tumor. One such assay involves contacting tumor cells with a binding agent. The bound binding agent may then be detected directly or indirectly via a reporter group. Such binding agents may also be used in histological applications. Alternatively, polynucleotide probes may be used within such applications.

As noted above, to improve sensitivity, multiple colon tumor protein markers may be assayed within a given sample. It will be apparent that binding agents specific for different proteins provided herein may be combined within a single assay. Further, multiple primers or probes may be used concurrently. The selection of tumor protein markers may be based on routine experiments to determine combinations that results in optimal sensitivity. In addition, or alternatively, assays for tumor proteins provided herein may be combined with assays for other known tumor antigens.

Diagnostic Kits

The present invention further provides kits for use within any of the above diagnostic methods. Such kits typically comprise two or more components necessary for performing a diagnostic assay. Components may be compounds, reagents, containers and/or equipment. For example, one container within a kit may contain a monoclonal antibody or fragment thereof that specifically binds to a colon tumor protein. Such antibodies or fragments may be provided attached to a support material, as described above. One or more additional containers may enclose elements, such as reagents or buffers, to be used in the assay. Such kits may also, or alternatively, contain a detection reagent as described above that contains a reporter group suitable for direct or indirect detection of antibody binding.

Alternatively, a kit may be designed to detect the level of mRNA encoding a colon tumor protein in a biological sample. Such kits generally comprise at least one oligonucleotide probe or primer, as described above, that hybridizes to a polynucleotide encoding a colon tumor protein. Such an oligonucleotide may be used, for example, within a PCR or hybridization assay. Additional components that may be present within such kits include a second oligonucleotide and/or a diagnostic reagent or container to facilitate the detection of a polynucleotide encoding a colon tumor protein.

The following Examples are offered by way of illustration and not by way of limitation.

### EXAMPLES

### Example 1

### Isolation and Characterization 0f Colon Tumor Polypeptides by PCR-Based Subtraction and Microarray Analysis

A cDNA library was constructed in the PCR2. 1 vector (Invitrogen, Carlsbad, Calif.) by subtracting a pool of three colon tumors with a pool of normal colon, spleen, brain, liver, kidney, lung, stomach and small intestine using PCR subtraction methodologies (Clontech, Palo Alto, Calif.). The subtraction was performed using a PCR-based protocol, which was modified to generate larger fragments. Within this protocol, tester and driver double stranded cDNA were separately digested with five restriction enzymes that recognize six-nucleotide restriction sites (MluI, MscI, PvuII, SalI and Stul). This digestion resulted in an average cDNA size of 600 bp, rather than the average size of 300 bp that results from digestion with RsaI according to the Clontech protocol. This modification did not affect the subtraction efficiency. Two tester populations were then created with different adapters, and the driver library remained without adapters.

The tester and driver libraries were then hybridized using excess driver cDNA. In the first hybridization step, driver was separately hybridized with each of the two tester cDNA populations. This resulted in populations of (a) unhybridized tester cDNAs, (b) tester cDNAs hybridized to other tester cDNAs, (c) tester cDNAs hybridized to driver cDNAs, and (d) unhybridized driver cDNAs. The two separate hybridization reactions were then combined, and rehybridized in the presence of additional denatured driver cDNA. Following this second hybridization, in addition to populations (a) through (d), a fifth population (e) was generated in which tester cDNA with one adapter hybridized to tester cDNA with the second adapter. Accordingly, the second hybridization step resulted in enrichment of differentially expressed sequences which could be used as templates for PCR amplification with adaptor-specific primers.

The ends were then filled in, and PCR amplification was performed using adaptor-specific primers. Only population (e), which contained tester cDNA that did not hybridize to driver cDNA, was amplified exponentially. A second PCR amplification step was then performed, to reduce background and further enrich differentially expressed sequences.

This PCR-based subtraction technique normalizes differentially expressed cDNAs so that rare transcripts that are over-expressed in colon tumor tissue may be recoverable. Such transcripts would be difficult to recover by traditional subtraction methods.

To characterize the complexity and redundancy of the subtracted library, 96 clones were randomly picked and 65 were sequenced, as previously described. These sequences were further characterized by comparison with the most recent Genbank database (April, 1998) to determine their degree of novelty. No significant homologies were found to 21 of these clones, hereinafter referred to as 11092, 11093, 11096, 11098, 11103, 11174, 11108, 11112, 11115, 11117, 11118, 11134, 11151, 11154, 11158, 11168, 11172, 11175, 11184, 11185 and 11187. The determined cDNA sequences for these clones are provided in SEQ ID NO: 48, 49, 52, 54, 59, 60, 65-69, 79, 89, 90, 93, 99-101 and 109-111, respectively.

Two-thousand clones from the above mentioned cDNA subtraction library were randomly picked and submitted to a round of PCR amplification. Briefly, 0.5 μl of glycerol stock solution was added to 99.5 μl of pcr MIX (80 μl H₂O, 10 μl 10×PCR Buffer, 6 μl 25 mM MgCl₂, 1 μl 10 mM dNTPs, 1 μl 100 mM M13 forward primer (CACGACGTTGTAAAACGACGG), 1 μl 100 mM M13 reverse primer (CACAGGAAACAGCTATGACC)), and 0.5 μl 5 μ/ml Taq polymerase (primers provided by (Operon Technologies, Alameda, Calif.). The PCR amplification was run for thirty cycles under the following conditions: 95° C. for 5 min., 92° C. for 30 sec., 57° C. for 40 sec., 75° C. for 2 min. and 75° C. for 5 minutes.

mRNA expression levels for representative clones were determined using microarray technology (Synteni, Palo Alto, Calif.) in colon tumor tissues (n=25), normal colon tissues (n=6), kidney, lung, liver, brain, heart, esophagus, small intestine, stomach, pancreas, adrenal gland, salivary gland, resting PBMC, activated PBMC, bone marrow, dendritic cells, spinal cord, blood vessels, skeletal muscle, skin, breast and fetal tissues. The number of tissue samples tested in each case was one (n=1), except where specifically noted above; additionally, all the above-mentioned tissues were derived from humans. The PCR amplification products were dotted onto slides in an array format, with each product occupying a unique location in the array. mRNA was extracted from the tissue sample to be tested, and fluorescent-labeled cDNA probes were generated by reverse transcription according to the protocol provided by Synteni. The microarrays were probed with the labeled cDNA probes, the slides scanned, and fluorescence intensity was measured. This intensity correlates with the hybridization intensity.

One hundred and forty nine clones showed two or more fold over-expression in the colon tumor probe group as compared to the normal tissue probe group. These cDNA clones were further characterized by DNA sequencing with a Perkin Elmer/Applied Biosystems Division Automated Sequencer Model 373A and/or Model 377 (Foster City, Calif.). These sequences were compared to known sequences in the most recent GenBank database. No significant homologies to human gene sequences were found in forty nine of these clones, represented by the following sixteen cDNA consensus sequences: SEQ ID NO: 2, 8, 15, 16, 22, 24, 30, 32-34, 36, 38, 40, 41, 46 and 47, hereinafter referred to as Contig 2, 8, 13, 14, 20, 23, 29, 31, 35, 32, 36, 38, 41, 42, 50 and 51, respectively). Contig 29 (SEQ ID NO: 30) was found to be a Rat GSK-3-β-interacting protein Axil homolog. Also, Contigs 31 and 35 (SEQ ID NO: 32 and 33, respectively) were found to be a Mus musculus GOB-4 homolog. The determined cDNA sequences of SEQ ID NO: 1, 3-7, 9-14, 17-21, 23, 25-29, 31, 35, 37, 39, 42-45, 50, 51, 53, 55-58, 61-64, 70-78, 80-88, 91, 92, 94-98, 102-108 and 112 were found to show some homology to previously identified genes sequences.

Microarray analysis demonstrated Contig 2 (SEQ ID NO: 2) showed over-expression in 34% of colon tumors tested, as well as increased expression in normal pancreatic tissue, with no over-expression in normal colon tissues. Upon further analysis, Contigs 2, 8 and 23 were found to share homology to the known gene GW112. Contigs 4, 5, 9 and 52 showed homology to carcinoembryonic antigen (SEQ ID NO: 3, 4, 5 and 6, respectively). A representative sampling of these fragments showed over-expression in 85% of colon tumors, with over-expression in normal bone marrow and 3/6 normal colon tissues. Contig 6 (SEQ ID NO: 7), showing homology to the known gene sequence for villin, and was over-expressed in about half of all colon tumors tested, with a limited degree of low level over-expression in normal colon. Contig 12 (SEQ ID NO: 14), showing homology to Chromosome 17, clone hRPC.1171_I_{—}10, also referred to as C798P, was over-expressed in approximately 70% of colon tumors tested, with low over-expression in 1/6 normal colon samples. Contig 14, also referred to as 14261 (SEQ ID NO: 16), showing no significant homology to any known gene, showed over-expression in 44% of colon tumors tested, with low level expression in half of normal colon tissues, as well as small intestine and pancreatic tissue. Contig 18 (SEQ ID NO: 21), showing homology to the known gene for L1-cadherin, showed over-expression in approximately half of colon tumors and low level over-expression in 3/6 normal colon tissues tested. Contig 22 (SEQ ID NO: 23), showing homology to Bumetanide-sensitive Na—K—Cl cotransporter was over-expressed in 70% of colon tumors and no over-expression in all normal tissues tested. Contig 25 (SEQ ID NO: 25), showing homology to macrophage inflammatory protein-3α, was over-expressed in over 40% of colon tumors and in activated PBMC. Contigs 26 and 48 (SEQ ID NOS: 25 and 26), showing homology to the sequence for laminin, was over-expressed in 48% of colon tumors and with low over-expression in stomach tissue. Contig 28 (SEQ ID NO: 29), showing homology to the known gene sequence for Chromosome 16 BAC clone CIT987SK-A-363E6, was over-expressed in 33% of colon tumors tested with normal stomach and 2/6 normal colon tissues showing low level over-expression. Contigs 29, 31 and 35 (SEQ ID NOS: 30, 32 and 33, respectively), also referred to as C751P, an unknown sequence showing limited and partial homology to Rat GSK-3p-interacting protein Axil homolog and Mus musculus GOB-4 homolog, was over-expressed in 74% of colon tumors and no over-expression in all normal tissues tested. Contig 34 (SEQ ID NO: 35), showing homology to the known sequence for desmoglein 2, was over-expressed in 56% of colon tumors and showed low level over-expression in 1/6 normal colon tissues. Contig 36 (SEQ ID NO: 36), an unknown sequence also referred to as C793P, showed over-expression in 30% of colon tumor tissues tested. Contig 37 and 14287.2 (SEQ ID NOS: 37 and 116), an unknown sequence, but with limited (89%) homology to the known sequence for putative transmembrane protein was over-expressed in 70% of colon tumors, as well as in normal lung tissue and 3/6 normal colon tissues tested. Contig 38, also referred to as C796P and 14219 (SEQ ID NO: 38), showing no significant homology to any known gene, was over-expressed in 38% in colon tumors and no elevated over-expression in any normal tissues. Contig 41 (SEQ ID NO: 40), also referred to as C799P and 14308, an unknown sequence showing no significant homology to any known gene, was over-expressed in 22% of colon tumors. Contig 42, (SEQ ID NO: 41), also referred to as C794P and 14309, an unknown sequence with no significant homology to any known gene, was over-expressed in 63% of colon tumors tested, as well as in 3/6 normal colon tissues. Contig 43 (SEQ ID NO: 42), showing homology to the known sequence for Chromosome 1 specific transcript KIAA0487 was over-expressed in 85% of colon tumors tested and in normal lung and 4/6 normal colon tissues. Contig 49 (SEQ ID NO: 45), showing homology to the known sequence for pump-1, was over-expressed in 44% of colon tumors and no over-expression in all normal tissues tested. Contig 50 (SEQ ID NO: 46), also referred to as C792P and 18323, showing no significant homology to any known gene, was over-expressed in 33% of colon tumors with no detectable over-expression in any normal tissues tested. Contig 51 (SEQ ID NO: 47), also referred to as C795P and 14317 was over-expressed in 11% of colon tumors.

Additional microarray analysis yielded seven clones showing two or more fold over-expression in the colon tumor probe group as compared to the normal tissue probe group. Three of these clones demonstrated particularly good colon tumor specificity, and are represented by SEQ ID NO: 115, 116 and 120. Specifically, SEQ ID NO: 115, referred to as C791P or 14235, which shows homology to the known gene sequence for H. sapiens chromosome 21 derived BAC containing ets-2 gene, was over-expressed in 89% of colon tumors tested and in 5/6 normal colon tissues, as well as over-expressed at low levels in normal lung and activated PBMC. Microarray analysis for SEQ ID NO: 116 is discussed above. SEQ ID NO: 120, referred to as 14295, showing homology to the known gene sequence for secreted cement gland protein XAG-2 homolog, was over-expressed in 70% of colon tumors and in 5/6 normal colon tissues, as well as low level over-expression in normal small intestine, stomach and lung. All clones showing over-expression in colon tumor were sequenced and these sequences compared to the most recent Genbank database (Feb. 12, 1999). Of the seven clones, three contained sequences that did not share significant homology to any known gene sequences, represented by SEQ ID NO: 116, 117 and 119. To the best of the inventors' knowledge, none of these sequences have been previously shown to be present in colon. The determined cDNA sequences of the remaining clones (SEQ ID NO: 113-115 and 120) were found to show some homology to previously identified genes.

Further analysis identified a clone which was recovered several times by PCR subtraction and by expression screening using a mouse anti-scid antiserum. The determined full length cDNA sequence for this clone is provided in SEQ ID NO: 121, with the corresponding predicted amino acid sequence being provided in SEQ ID NO: 122. This clone is homologous with the known gene Beta IG-H3, as disclosed in U.S. Pat. No. 5,444,164. Microarray analysis demonstrated this clone to be over-expressed in 75 to 80% of colon tumors tested (n=27), with no over-expression in normal colon samples (n=6), but with some low level over-expression in other normal tissues tested.

Further analysis of the PCR-subtraction library described above led to the isolation of longer cDNA sequences for the clones of SEQ ID NO: 30, 115, 46, 118, 41, 47, 38, 113, 14 and 40 (known as C751P, C791P, C792P, C793P, C794P, C795P, C796P, C797P, C798P and C799P, respectively). These determined cDNA sequences are provided in SEQ ID NO: 123-132, respectively. Additional sequences for the clones C794P and C799P are shown in SEQ ID NO: 683 and 684, respectively, and the predicted amino acid sequences are shown in SEQ ID NO: 685 and 686, respectively. Still further sequences for the clones C794P and C799P are shown in SEQ ID NO: 691 and 690, respectively, and to the predicted amino acid sequence as shown in SEQ ID NO: 693 and 692, respectively.

Using PCR subtraction methodology described above with minor modifications, transcripts from a pool of three moderately differentiated colon adenocarcinoma samples were subtracted with a set of transcripts from normal brain, pancreas, bone marrow, liver, heart, lung, stomach and small intestine. Modifications of the above protocol were included at the cDNA digestion steps and in the tester to drive hybridization ratios. In a first subtraction, the restriction enzymes PvuII, DraI, MscI and StuI were used to digest cDNAs, and the tester to driver ratio was 1:40, as suggested by Clontech. In a second subtraction, DraI, MscI and StuI were used for cDNA digestion and a tester to driver ratio of 1:76 was used. Following the PCR amplification steps, the cDNAs were clones into pCR2.1 plasmid vector. The determined cDNA sequences of 167 isolated clones are provided in SEQ ID NO: 205-371. These sequences were compared to sequences in the public databases as described above. The sequences of SEQ ID NO: 205, 207, 210-212, 214, 215, 218, 224-226, 228, 233, 234, 236, 238, 241, 242, 245, 246, 248, 250, 253, 254, 256, 259, 260, 262, 263, 266, 267, 270-273, 279, 282, 291, 293, 294, 298, 300, 302, 303, 310-313, 315, 317, 320, 322, 324, 332-335, 345, 347, 356, 358, 361, 362, 366, 369 and 371 were found to show some homology to previously identified ESTs. The remaining sequences were found to show some homology to previously identified genes.

Using the PCR subtraction technology described above, a cDNA library from a pool of primary colon tumors was subtracted with a cDNA library prepared from normal tissues, including brain, bone marrow, kidney, heart, lung, liver, pancreas, small intestine, stomach and trachea. The determined cDNA sequences for 90 clones isolated in this subtraction are provided in SEQ ID NO: 372-461. Comparison of these sequences with those in the public databases as described above, revealed no homologies to the sequences of SEQ ID NO: 426, 445 and 453. The sequences of SEQ ID NO: 372-378, 380-404, 406, 409-417, 419-423, 425, 427-429, 433-436, 438-441, 443, 446-451, 454, 455 and 457-461 showed some homology to previously identified genes, while the sequences of SEQ ID NO: 379, 405, 407, 408, 418, 424, 430-432, 437, 442, 444, 452 and 456 showed some homology to previously isolated ESTs.

Using the PCR subtraction methodology described above, a cDNA library prepared from a pool of metastatic colon tumors was subtracted with cDNA from a pool of normal tissues, namely brain, heart, lung, lymph nodes, PBMC, pancreas, small intestine and stomach. The determined cDNA sequences for 82 clones isolated from the subtracted library are provided in SEQ ID NO: 487-568 (referred to as contigs 1-56 and 58-83, respectively). The sequences of SEQ ID NO: 487, 489, 490, 493-496, 499, 501-509, 511-518, 520-526, 529-542, 544, 546, 548-552, 554, 555, 557, 558, 560, 562, 563, 566 and 567 showed some homology to previously identified gene sequences. The sequences of SEQ ID NO: 488, 491, 492, 497, 498, 500, 510, 519, 527, 528, 543, 545, 547, 553, 559, 564, 564 and 568 showed some homology to previously isolated ESTs.

### Example 2

### Isolation of Tumor Polypeptides Using SCID Mouse-Passaged Tumor RNA

This Example discloses the preparation of antisera against shed/secreted antigens from SCID mice bearing human colon tumors. These antisera may be useful, for example, in the screening of cDNA libraries made from the original human colon tumors for secreted antigens that may, in turn, be useful for identification of therapeutic and/or diagnostic candidates.

Human colon tumor antigens were obtained using SCID mouse passaged colon tumor RNA as follows. Human colon tumor was implanted in SCID mice and harvested, as described in patent application Ser. No. 08/556,659 filed Nov. 13, 1995 U.S. Pat. No. 5,986,170. First strand cDNA was synthesized from poly A+ RNA from three SCID mouse-passaged colon tumors using a Lambda ZAP Express cDNA synthesis kit (Stratagene). The reactions were pooled and digested with RNase A, T1 and H to cleave the RNA and then treated with NaOH to degrade the RNA. The resulting cDNA was annealed with biotinylated (Vector Labs, Inc., Burlingame, Calif.) cDNA from a normal resting PBMC plasmid library (constructed from Superscript plasmid System, Gibco BRL), and subtracted with streptavidin by phenol/chloroform extraction. Second strand cDNA was synthesized from the subtracted first strand cDNA and digested with S1 nuclease (Gibco BRL). The cDNA was blunted with Pfu polymerase and EcoRI adaptors (Stratagene) were ligated to the ends. The cDNA was phosphorylated with T4 polynucleotide kinase, digested with restriction endonuclease XhoI, and size selected with Sephacryl S-400 (Sigma). Fractions were pooled, ligated to Lambda ZAP Express arms (Stratagene) and packaged with Gigapack Gold III extract (Stratagene). Random plaques were picked, phagemid was excised, transformed into XLOLR cells (Stratagene) and resulting plasmid DNA (Qiagen Inc., Valencia, Calif.) was sequenced as described above.

The determined cDNA sequences for 17 clones isolated as described above are provided in SEQ ID NO: 133-151, wherein 133 and 134 represent partial sequences of a clone referred to as CoSub-3 and SEQ ID NO: 135 and 136 represent partial sequences of a clone referred to as CoSub-13. These sequences were compared with those in the public databases as described above. The sequences of SEQ ID NO: 139 and 149 showed no significant homologies to any previously identified sequences. The sequences of SEQ ID NO: 138, 140, 141, 142, 143, 148 and 149 showed some homology to previously isolated expressed sequence tags (ESTs). The sequences of SEQ ID NO: 133-137, 144-147, 150 and 151 showed some homology to previously isolated gene sequences.

The determined cDNA sequences for an additional 46 clones isolated as described above, are provided in SEQ ID NO: 569-616, wherein SEQ ID NO: 573 and 574 represent the 3′ and 5′ determined cDNA sequences, respectively, for clone CS1-106, and SEQ ID NO: 579 and 580 represent the determined 3′ and 5′ cDNA sequences, respectively, for clone CS1-124. Comparison of the isolated sequences with those in the public databases revealed no significant homologies to the sequences of SEQ ID NO: 580, 585, 610 and 613. The sequences of SEQ ID NO: 569, 574-577, 584, 587, 592, 595, 598, 603 and 608 showed some homology to previously isolated ESTs, while the sequences of SEQ ID NO: 570-573, 578, 581-583, 586, 588-591, 593, 594, 596, 597, 599-602, 604-607, 609, 611, 612 and 514-616 showed some homology to previously isolated gene sequences.

### Example 3

### Use of Mouse Antisera to Identify DNA Sequences Encoding Colon Tumor Antigens

This example illustrates the isolation of cDNA sequences encoding colon tumor antigens by screening of colon tumor cDNA libraries with mouse anti-tumor sera.

A cDNA expression library was prepared from SCID mouse-passaged human colon tumor poly A+ RNA using a Stratagene (La Jolla, Calif.) Lambda ZAP Express kit, following the manufacturer's instructions. Sera was obtained from the colon tumor-bearing SCID mouse. This serum was injected into normal mice to produce anti-colon tumor serum. Approximately 600,000 PFUs were screened from the unamplified library using this antiserum. Using a goat anti-mouse IgG-A-M (H+L) alkaline phosphatase second antibody developed with NBT/BCIP (BRL Labs.), positive plaques were identified. Phage was purified and phagemid excised for several clones with inserts in a pBK-CMV vector for expression in prokaryotic or eukaryotic cells.

The determined cDNA sequences for 46 of the isolated clones are provided in SEQ ID NO: 152-197. The predicted amino acid sequences for the cDNA sequences of SEQ ID NO: 187, 188, 189, 190, 194, 195 and 197 are provided in SEQ ID NO: 198-204, respectively. The determined cDNA sequences were compared with those in the public database as described above. The sequences of SEQ ID NO: 156, 168, 184, 189, 192 and 196 showed some homology to previously isolated ESTs. The sequences of SEQ ID NO: 152-155, 157-167, 169-182, 183, 185-188, 190, 194, 195 and 197 showed some homology to previously identified genes.

The determined cDNA sequences for an additional eleven clones isolated as described above, are provided in SEQ ID NO: 617-627. Comparison of these sequences with those in the public database as described above revealed no known homologies to SEQ ID NO: 621 and 623. The sequences of SEQ ID NO: 622 and 626 were found to show some homology to previously isolated ESTs, while the sequences of SEQ ID NO: 617-620, 624, 625 and 627 showed some homology to previously identified genes.

In further studies, a cDNA library was prepared from SCID-mouse grown colon tumors and screened with mouse anti-SCID serum as described above. Briefly first strand cDNA was synthesized from poly A+ RNA from three SCID mouse-grown human colon tumors using a Lambda ZAP Express cDNA synthesis kit (Stratagene). The reactions were pooled and digested with RNase A, T1 and H to cleave the RNA and then treated with NaOH to degrade the RNA. The cDNA was annealed with biotinylated cDNA from a normal resting PBMC plasmid library (constructed from Superscript plasmid system; Gibco BRL) and subtracted with streptavidin by phenol/chloroform extraction. Second strand cDNA was synthesized from the subtracted first strand cDNA and digested with S1 nuclease. The cDNA was blunted with Pfu polymerase and EcoRI adaptors were ligated to the ends. The cDNA was phosphorylated with T4 polynucleotide kinase, digested with restriction endonuclease XhoI, and size selected with Sephacryl S-400 (Sigma). Fractions were pooled, ligated to Lambda ZAP Express arms (Stratagene) and packaged with Gigapack Gold III extract (Stratagene). The resulting library was screened with a mouse antiserum raised against serum from SCID mice containing human colon tumors, including the three tumors used to prepare the cDNA libraries.

The determined cDNA for one clone isolated using this procedure is provided in SEQ ID NO: 630. This clone was found to show homology to a previously identified gene. The amino acid sequence encoded by the clone of SEQ ID NO: 630 is provided in SEQ ID NO: 631.

In subsequent studies, an additional cDNA library was prepared from a SCID-passaged human colon tumor and screened with a mouse antiserum raised against serum from the SCID mouse containing the colon tumor. The determined cDNA sequences for 51 clones isolated in these studies are provided in SEQ ID NO: 632-682. Comparison of these sequences with those in the public databases revealed no significant homologies to the sequences of SEQ ID NO: 648 and 668. The sequence of SEQ ID NO: 642 showed some homology to previously isolated ESTs. The sequences of SEQ ID NO: 632-641, 643-647, 649-667 and 669-682 were found to show some homology to previously identified genes. SEQ ID NO: 684 and SEQ ID NO: 690 showed homology to human NADH/NADPH thyroid oxidase p138-tox mRNA.

### Example 4

### Isolation and Characterization of Colon Tumor Polypeptides by Conventional Subtraction

wo cDNA libraries were constructed and used to create a subtracted cDNA library as follows.

Using the GibcoBRL Superscript Plasmid System with minor modifications, two cDNA libraries were created. The first library, referred to as CTCL, was prepared from a pool of mRNA samples from three colon adenocarcinoma tissue samples. Two of the samples were described as Duke's stage C. and one as Duke's stage B. All three samples were grade III in histological status. A second library (referred to as DriverLibpcDNA3.1+) was prepared from a pool of normal tissues, namely liver, pancreas, skin, bone marrow, resting PBMC, stomach and brain. Both libraries were prepared using the manufacturer's instructions with the following modifications: an EcoRI-NotI 5′ cDNA adapter was used instead of the provided reagent; the vector pCDNA3.1 (+) (Invitrogen) was substituted for the pSPORT vector; and the ligated DNA molecules were transformed into ElectroMaxDH10B electrocompetent cells. Clones from the libraries were analyzed by restriction digest and sequencing to determine average insert size, quality of the library and complexity of the library. DNA was prepared from each library and digested.

The driver DNA was biotinylated and hybridized with the colon library tester DNA at a ratio of 10:1. After two rounds of hybridizations, streptavidin incubations and extractions, the remaining colon cDNAs were size-selected by column chromatography and cloned into the pCMV-Script vector from Stratagene. Clones from this subtracted library (referred to as CTCL-S1) were characterized as described above for the unsubtracted libraries.

The determined cDNA sequences for 20 clones isolated from the CTCL-S 1 library are provided in SEQ ID NO: 462-479, 628 and 629. Comparison of these sequences with those in the public databases, as described above, revealed no significant homologies to the sequences of SEQ ID NO: 476, 477 and 479. The remaining sequences showed some homology to previously identified genes.

In further studies, a cDNA library was prepared from a pool of mRNA from three metastatic colon adenocarcinomas derived from liver tissue samples. All samples were described as Duke's stage D. Conventional subtraction was performed as described above, using the DriverLibpcDNA3.1+ library described above as the driver. The resulting subtracted library (referred to as CMCL-S1) was characterized by isolating a set of clones for restriction analysis and sequencing.

The determined cDNA sequences for 7 clones isolated from the CMCL-S1 library are provided in SEQ ID NO: 480-486. Comparison of these sequences with those in the public databases revealed no significant homologies to the sequence of SEQ ID NO: 483. The sequences of SEQ ID NO: 480-482 and 484-486 were found to show some homology to previously identified genes.

### Example 5

### Expression of RA12-C884P Fusion Protein in E. COLI

PCR was used to amplify the C884P cDNA (SEQ ID NO: 1052) using the primers below and a colon cDNA library (648A). The PCR product was cloned to pCRX2 at EcoR I and Xho I site, downstream of 130 amino acid segment of the Ra12 protein. Three clones (Clone Identifier NO: 61698, 61699 and 61700) were all confirmed by DNA sequence. The cDNA encoding the Ra12-C884P fusion is set forth in SEQ ID NO: 1084 and the corresponding predicted amino acid sequence is disclosed in SEQ ID NO: 1085.

The following primers were used for PCR amplification of C884P:

080300-A (Primer Identifier 7839) (SEQ ID NO: 1088):

5′-cgagcgaattcatatgggtacgagtaagcaatg-3′ (5′ Eco RI site).

080300-B (Primer ID 7840) (SEQ ID NO: 1089):

5′-gcgatgcctcgagttatttgttcccgatctggc-3′ (3′ Xho I site).

The predicted protein has the following features: Protein sequence information: Molecular Weight 36466.18 Daltons; 344 Amino Acids; 21 Strongly Basic (+) Amino Acids (K,R); 27 Strongly Acidic (−) Amino Acids (D,E); 144 Hydrophobic Amino Acids (A,I,L,F,W,V); 88 Polar Amino Acids (N,C,Q,S,T,Y); 6.165 Isolectric Point; −4.709 Charge at PH 7.0.

Mini-induction screening of Ra12-C884P in numerous E. coli hosts, using various temperature, culture media and concentrations of IPTG, showed a protein band at expected MW of 36.5 kDa, visible by western blot but not by SDS-PAGE/Coomassie staining. The best expression condition was in BLR(DE3)pLysS host and recombinant protein induced by IPTG at 30° C. for 2 hours.

### Example 6

### Expression of RA12-C888P Fusion Protein in E. COLI

PCR was used to amplify the ORF of C888P (SEQ ID NO: 1076; Clone Identifier No: 37983) from a colon cDNA library (648A) using the following primers:

Sense Primer -080300-C (Primer ID7841) (SEQ ID 1090):

5′-gcatgcatgcggccgcacaagaggggaagtttagtgg-3′ (5′ Not I site)

Antisense Primer-080300-D (Primer ID7842) (SEQ ID NO: 1091):

5′-gcgatgcctcgagtcagcttctcagaggtttgacttc-3′ (3′ Xho I site)

The PCR product was cloned into the pZeroBlunt vector (Invitrogen, Carlsbad, Calif.) and confirmed by DNA sequencing. The C888P insert was cut with Not I and Xho I and subcloned into pCRX2 linearized with the same two enzymes. A clone (clone identifier 66481) was confirmed by DNA sequencing. The cDNA sequence encoding the Ra12-C888P fusion protein (R.C888P) is disclosed in SEQ ID NO: 1086 and the corresponding predicted amino acid sequence is disclosed in SEQ ID NO: 1087.

The predicted R.C888P protein has the following features: molecular weight 104429.77 Daltons; 958 Amino Acids; 78 Strongly Basic(+) Amino Acids (K,R); 116 Strongly Acidic(−) Amino Acids (D,E); 331 Hydrophobic Amino Acids (A,I,L,F,W,V); 260 Polar Amino Acids (N,C,Q,S,T,Y); 4.985 Isolectric Point: −34.173 Charge at PH 7.0

Multiple “mini” expression screens were performed to determine the optimal induction conditions. E. coli expression hosts were tested in various culture conditions, with notable full length expression but also multiple breakdown products. To test for reduction of breakdown product and maximize clean recombinant production, Tuner (DE3) C+RIL and C+RP cells were induced at varied IPTG concentrations. Coomassie stained SDS-PAGE showed a specifically induced band at about 100 kD and western blot confirmed with anti-6×his tag Ab. Western blot also showed no breakdown products at 0.1 mM IPTG concentration. Tuner (DE3) C+RP expression host grows best (high cell density) with optimal expression of R.C888P in 2×YS media at 25° C. induced with 0.1 mM IPTG at 25° C. for 3 hr.

### Example 7

### Database Analysis of C794P CDNA Encoding Colon Tumor Protein

Database searches were performed with C794P (SEQ ID NOs: 41, 127, 683, 691). Sequence similarity was seen to cDNA FLJ20063 and the RECC gene (SEQ ID NO: 1092). This RECC sequence is longer than the FLJ20063 cDNA, containing additional sequence at the 5′ end. The RECC sequence contains an ORF encoding a predicted protein of 512 amino acids (SEQ ID NO: 1093). This RECC protein shares similarity with mouse cell surface antigen 114/A10. This mouse antigen was found in a hematopoetic progenitor cell line and is described as an intergral membrane protein with serine/threonine repeats at the N-terminus and 3 EGF-like cysteine repeats. It was also detected in a leukemia cell line and IL-3-dependent cell lines (J. Biol. Chem. 1989. 264(11):6509-6514).

Results from the PSORTII protein analysis program (University of California-Berkeley, Berkeley, Calif.) suggest that RECC is a cell surface protein containing 1 transmembrane domain. PSORTII predicts a cleavable signal peptide from amino acids 1-17, one transmembrane domain, a C-terminal cytoplasmic tail, amino acids 446-512, and a 44.4% likelihood of extracellular localization. The IDENTIFY analysis program (Stanford University, Palo Alto, Calif.) predicts four EGF-type signatures CACVPGY and one rhesus blood group protein signature at stringency of one in 10⁶ (no false positives expected).

### Example 8

### Microarray Expression Analysis of CDNAS Encoding Colon Tumor Polypeptides Isolated From a Serological Expression Library

mRNA expression levels of cDNA clones identified in the serological expression library described in Example 3 were analyzed by microarray as described in Example 1. Those clones with expression levels 2 fold or higher in tumor tissues as compared to normal tissues were further characterized by comparison with the most recent Genbank database. Those sequences that showed some degree of similarity to sequences in the database are summarized in Table 1. Those sequences that showed no significant similarity to known sequences in the database are summarized in Table 2. Disclosed herein are several full length cDNA and predicted amino acid sequences for genes provided in the results of Genbank searches. The full length cDNA sequences are set forth in SEQ ID NOs: 1096-1099, 1101, 1103-1106, and 1111-1114, and the full length predicted amino acid sequences are set forth in 1102, 1107-1110, and 1115-1118. The clone, CT2-222, showed the most similarity to a murine gene (disclosed in SEQ ID NOs: 1113 (cDNA) and 1117 (amino acid)). The human homolog of this gene is disclosed in SEQ ID NOs: 1114 (cDNA) and 1118 (amino acid).

TABLE 1
Microarray and Genbank search analysis of cDNAs isolated from a serological expression library:
Sequences that showed some similarity to sequences in Genbank

| | | | | Ratio of Median Values | | | | | | |
|---|---|---|---|---|---|---|---|---|---|---|
| | | | | Tumor/Normal Tissue | | | | | | |
| SEQ | Clone | Sequence | | | | | | | | |
| ID NO: | Name | Identifier | GenBank ID | T/N | DA/N | DB/N | DC/N | DD/N | Met* | T/N-C |
| 171, | CT-53 | 24132 | Galectin-4 (secreted) | 3.2 | 2.4 | 5.4 | 2.1 | 4.5 | | 3.5 |
| 1096 | | | | | | | | | | |
| 181, | CT-126 | 25527 | GPI-anch. Surface mark 1 | — | — | 2.3 | — | — | | 2.4 |
| 1097 | | | | | | | | | | |
| 183, | CT-140 | 25537 | hAG-2/C (hAG-1)(secreted) | 7.6 | 4.2 | 10.6 | 3.8 | 8.9 | | 9.5 |
| 1098 | | | | | | | | | | |
| 186, | CT-148 | 25544 | Glucose Phosp. Isomerase | — | — | 2.1 | — | — | | — |
| 1099 | | | | | | | | | | |
| | CT2-104 | 41096 | beta IG-H3 (secreted) | 2.4 | 2 | 2.5 | 2.1 | 3.5 | | 2.4 |
| 665, | CT2-167 | 39819 | Keratin 19 (shed) | 3.4 | — | 3.7 | 2.3 | 3.9 | | 5.2 |
| 1111, | | | | | | | | | | |
| 1115 | | | | | | | | | | |
| 630 | CSE-2 | 35997 | Unkn (hAG-3) (secreted) | 5.6 | 5.3 | 6 | — | 6.6 | | 5.7 |
| 571, | CS1-104 | 31409 | Squalene epoxidase | — | — | 2.4 | — | 3.1 | | — |
| 1103, | | | | | | | | | | |
| 1107 | | | | | | | | | | |
| 573, 574, | CS1-106 | 31364 | Mad p2 | 2.2 | — | 2.4 | — | 3.8 | | 2.2 |
| 1104, | | | | | | | | | | |
| 1108 | | | | | | | | | | |
| 578, | CS1-123 | 31396 | Epith-spec trans fac. ESE-1b | 3.9 | — | 4.4 | 2.4 | 6 | | 8.5 |
| 1105, | | | | | | | | | | |
| 1109 | | | | | | | | | | |
| 587, | CS-152 | 32214 | Hu. Regenerating gene type | | | | | | | |
| 1069, | | | IV | | | | | | | |
| 1070 | | | | | | | | | | |
| 591, | CS1-160 | 32222 | KSA; adenocarc-assoc Ag | — | — | 2 | — | 3.4 | | 2.7 |
| 1106, | | | | | | | | | | |
| 1110 | | | | | | | | | | |

| | | | | | |
|---|---|---|---|---|---|
| 675, | CT2-222 | 39848 | Mu. valosin containing pro.; | 1.24 | **Very low overexpression on 13/38 tumors, |
| 1113, | | | Hu. homolog | | and on 5/37 normals |
| 1117 | | | | | |
| 1101, | CT2-136; | 41099 | Hu. heat shock protein apg-2 | 1.08 | **Low to high overexpression on all tumors; Moderate |
| 1102 | (identi- | (24115) | | | OE in N. thymus, low OE in N.lung, pano., b.marrow, |
| (161) | cal to CT- | | | | skel.muscle, skin, aorta, heart, act.PBMC, fetal |
| | 17) | | | | |
| 1112, | CT2-147 | 41100 | Hu. Fc fragment of IgG | 1.87 | **Moderate OE in 4, low OE in 8 tumors; low |
| 1116, | | | binding protein (FCGBP) | | OE in 2, mod. OE in N.b.marrow, co |
| 172 | PACSINA-6 | (24595) | Hu. PACSIN2 | 1.57 | **Low-moderate OE in 34/37 tumors; Moderate OE in |
| | | | | | N.colon, lung, b.marrow, aorta, skin act.PBMC |

*Met ratio of means
**Clones were picked based upon their low overexpression in normals, not necessarily due to their levels of overexpression positives.

TABLE 2
Microarray and Genbank search analysis of cDNAs isolated from a serological expression library:
Sequences that showed no significant similarity to known sequences in Genbank

| | | | | Ratio of Median Values | | | | | | |
|---|---|---|---|---|---|---|---|---|---|---|
| | | | | Tumor/Normal Tissue | | | | | | |
| SEQ | Clone | Sequence | | | | | | | | |
| ID NO: | Name | Identifier | GenBank ID | T/N | DA/N | DB/N | DC/N | DD/N | Met* | T/N-C |
| 193, 623, | CT-594 | 27540 | Mus musculus 10 day old male pancreas | | | | | | 40 | |
| 1082, | | | cDNA (Mouse EST hits: 113 embryonic, fetal | | | | | | | |
| 1083 | | | newborn, etc) | | | | | | | |

| | | | | |
|---|---|---|---|---|
| 1100 | CT2-283 | 41103 | 1.59 | **Low OE in 35/37 tumors; low OE in N.colon, |
| | | | | b.marrow, skin, aorta |

*Met ratio of means
**Clones were picked based upon their low overexpression in normals, not necessarily due to their levels of overexpression positives.

### Example 9

### Northern and Real Time PCR Expression Analysis of the C799P Colon Tumor Antigen

Searches of the C799P sequence (which relates to Clone Identifier No 14308 and the cDNA sequences disclosed in SEQ ID NOs: 40, 132, 684, and 690; and the amino acid sequences disclosed in SEQ ID NOs: 686 and 692) were performed against Genbank, human EST and Geneseq databases. A match was found against the 6410 bp NADPH thyroid oxidase 2 (THOX2) mRNA (SEQ ID NO: 1094). An ORF of 1548 amino acids was identified (SEQ ID NO: 1095). PSORTII protein motif prediction analysis suggests the protein contains a cleavable signal peptide and 9 transmembrane domains.

mRNA expression levels were further analyzed using real-time PCR. Real-time PCR (see Gibson et al., Genome Research 6:995-1001, 1996; Heid et al., Genome Research 6:986-994, 1996) is a technique that evaluates the level of PCR product accumulation during amplification. This technique permits quantitative evaluation of mRNA levels in multiple samples. Briefly, niRNA was extracted from tumor and normal tissue and cDNA was prepared using standard techniques. Real-time PCR was performed using a Perkin Elmer/Applied Biosystems (Foster City, Calif.) 7700 Prism instrument. Matching primers and fluorescent probes were designed for C799P using the primer express program provided by Perkin Elmer/Applied Biosystems (Foster City, Calif.). Optimal concentrations of specific and control (e.g., β-actin) primers and probes were determined. To quantitate the amount of specific RNA in the samples (see Table 3), a standard curve was generated using a plasmid containing C799P. Standard curves were generated using the Ct values determined in the real-time PCR, which are related to the initial cDNA concentration used in the assay. Standard dilutions ranging from 10-10⁶ copies of the gene of interest are generally sufficient. In addition, a standard curve was generated for the control sequence. This permitted standardization of initial RNA content of the tissue samples to the amount of control for comparison purposes. The real-time data showed that C799P is overexpressed in 12 of 31 colon tumor samples when compared to expression seen in normal tissues. Some expression of C799P was observed in normal prostate, lung, pancreas, stomach, salivary gland, thyroid, and esophagus, although expression levels were markedly lower than those seen in colon tumor samples. Overexpression was also observed in lung and ovarian tumor samples. These data indicate that C799P has applicability in diagnostic and/or immunotherapeutic uses.

Northern analysis confirmed the real-time and earlier microarray data which showed C799P to be overexpressed in colon tumor.

### Example 10

### Full-Length Cloning of C884P Coding Sequence and Expression of C884P-HIS AND MAPS-C884P-HIS in HEK293T Cells

The full-length C884P protein coding sequence was cloned by RT-PCR. Standard RT-PCR was performed to clone the C884P ORF (including the signal sequence) into the TA cloning vector. The mRNA was isolated from a colon tumor, reverse transcribed to cDNA and used as template for PCR. The primers used for the RT-PCR have the following sequence:

C884P-HisTag-AW175: (sense primer) Id=10468 (SEQ ID NO: 1123)

5′ ggagctagcgccgccATGGCAGGTGTGAGTGCCTG

C884P-HisTag-AW176: (antisense primer) Id=10469 (SEQ ID NO: 1124)

5′ gccggatccTCAatggtgatggtgatggtgTTTGTTCCCGATCTGGCAATACAG

Six His residues were built into the 3′ primer (C884P-HisTag-AW176) immediately upstream of the stop codon. The cloned C884P gene was confirmed by DNA sequencing.

Full-length C884 with a C-terminal His tag was cloned into JA4304 at Nhe I and BamH I sites using standard molecular biology techniques (cDNA and amino acid sequences set forth in SEQ ID NOs: 1120 and 1122, respectively). Similarly, a fusion construct of MAPS-C884P-CHisTag was also made (cDNA and amino acid sequences set forth in SEQ ID NOs: 1119 and 1121, respectively). Both constructs were confirmed by DNA sequencing.

Different amounts (0.1 μg, 0.33 μg, 1 μg and 2 μg) of purified plasmids HEK293 cells. Cells were harvested 48 h later, and both the supernatant and cell lysate fractions were examined for the expression of C884P-CHisTag or MAPS-C884P-CHisTag proteins by western blots using anti-MAPS antibody and anti-C-terminal His tag antibody. No protein expression was observed by western blots in the supernatant fraction for all three transfections using either anti-MAPS antibody or anti C-terminal Histag antibody. With the anti-MAPS antibody, clean western bands were observed in a concentration dependent manner in the cell lysate transfected with MAPS-C884 fusion construct.

### Example 11

### Synthesis of Polypeptides

Polypeptides may be synthesized on a Perkin Elmer/Applied Biosystems Division 430A peptide synthesizer using FMOC chemistry with HPTU (O-Benzotriazole-N,N,N′, N′-tetramethyluronium hexafluorophosphate) activation. A Gly-Cys-Gly sequence may be attached to the amino terminus of the peptide to provide a method of conjugation, binding to an immobilized surface, or labeling of the peptide. Cleavage of the peptides from the solid support may be carried out using the following cleavage mixture: trifluoroacetic acid:ethanedithiol:thioanisole:water:phenol (40:1:2:2:3). After cleaving for 2 hours, the peptides may be precipitated in cold methyl-t-butyl-ether. The peptide pellets may then be dissolved in water containing 0.1% trifluoroacetic acid (TFA) and lyophilized prior to purification by C18 reverse phase HPLC. A gradient of 0%-60% acetonitrile (containing 0.1% TFA) in water (containing 0.1% TFA) may be used to elute the peptides. Following lyophilization of the pure fractions, the peptides may be characterized using electrospray or other types of mass spectrometry and by amino acid analysis.

From the foregoing it will be appreciated that, although specific embodiments of the invention have been described herein for purposes of illustration, various modifications may be made without deviating from the spirit and scope of the invention. Accordingly, the invention is not limited except as by the appended claims.

What is claimed:

1. An isolated polynucleotide comprising a sequence selected from the group consisting of:
(a) sequences provided in SEQ ID NOs: 1-121, 123-197, 205-630, 632-684, 686, 690-691, 694-1058, 1069, 1071-1076, 1082, 1084, 1086, 1092, 1094, 1096-1101, 103-1106, 1111-1114, 1119, and 1120;
(b) complements of the sequences provided in SEQ ID NOs: 1-121, 123-197, 205-630, 632-684, 686, 690-691, 694-1058, 1069, 1071-1076, 1082, 1084, 1086, 1092, 1094, 1096-1101, 1103-1106, 1111-1114, 1119, and 1120;
(c) sequences consisting of at least 20 contiguous residues of a sequence provided in SEQ ID NOs: 1-121, 123-197, 205-630, 632-684, 686, 690-691, 694-1058, 1069, 1071-1076, 1082, 1084, 1086, 1092, 1094, 1096-1101, 1103-1106, 1111-1114, 1119, and 1120;
(d) sequences that hybridize to a sequence provided in SEQ ID NOs: 1-121, 123-197, 205-630, 632-684, 686, 690-691, 694-1058, 1069, 1071-1076, 1082, 1084, 1086, 1092, 1094, 1096-1101, 1103-1106, 1111-1114, 1119, and 1120, under moderately stringent conditions;
(e) sequences having at least 75% identity to a sequence of SEQ ID NOs: 1-121, 123-197, 205-630, 632-684, 686, 690-691, 694-1058, 1069, 1071-1076, 1082, 1084, 1086, 1092, 1094, 1096-1101, 1103-1106, 1111-1114, 1119, and 1120;
(f) sequences having at least 90% identity to a sequence of SEQ ID NOs: 1-121, 123-197, 205-630, 632-684, 686, 690-691, 694-1058, 1069, 1071-1076, 1082, 1084, 1086, 1092, 1094, 1096-1101, 1103-1106, 1111-1114, 1119, and 1120; and
(g) degenerate variants of a sequence provided in SEQ ID NOs: 1-121, 123-197, 205-630, 632-684, 686, 690-691, 694-1058, 1069, 1071-1076, 1082, 1084, 1086, 1092, 1094, 1096-1101, 1103-1106, 1111-1114, 1119, and 1120.

2. An isolated polypeptide comprising an amino acid sequence selected from the group consisting of:
(a) sequences encoded by a polynucleotide of claim 1;
(b) sequences having at least 70% identity to a sequence encoded by a polynucleotide of claim 1;
(c) sequences having at least 90% identity to a sequence encoded by a polynucleotide of claim 1;
(d) sequences set forth in SEQ ID NOs: 122, 198-204, 631, 685, 687, 692, 693, 1059-1068, 1070, 1077-1081, 1083, 1085, 1087, 1093, 1095, 1102, 1107-1110, 1115-1118, 1121, and 1122;
(e) sequences having at least 70% identity to a sequence set forth in SEQ ID NOs: 122, 198-204, 631, 685, 687, 692, 693, 1059-1068, 1070, 1077-1081, 1083, 1085, 1087, 1093, 1095, 1102, 1107-1110, 1115-1118, 1121, and 1122; and
(f) sequences having at least 90% identity to a sequence set forth in SEQ ID NOs: 122, 198-204, 631, 685, 687, 692, 693, 1059-1068, 1070, 1077-1081, 1083, 1085, 1087, 1093, 1095, 1102, 1107-1110, 1115-1118, 1121, and 1122.

3. An expression vector comprising a polynucleotide of claim 1 operably linked to an expression control sequence.

4. A host cell transformed or transfected with an expression vector according to claim 3.

5. An isolated antibody, or antigen-binding fragment thereof, that specifically binds to a polypeptide of claim 2.

6. A method for detecting the presence of a cancer in a patient, comprising the steps of:
(a) obtaining a biological sample from the patient;
(b) contacting the biological sample with a binding agent that binds to a polypeptide of claim 2;
(c) detecting in the sample an amount of polypeptide that binds to the binding agent; and
(d) comparing the amount of polypeptide to a predetermined cut-off value and therefrom determining the presence of a cancer in the patient.

7. A fission protein comprising at least one polypeptide according to claim 2.

8. An oligonucleotide that hybridizes to a sequence recited in SEQ ID NOs: 1-121, 123-197, 205-630, 632-684, 686, 690-691, 694-1058, 1069, 1071-1076, 1082, 1084, 1086, 1092, 1094, 1096-1101, 1103-1106, 1111-1114, 1119, and 1120, under moderately stringent conditions.

9. A method for stimulating and/or expanding T cells specific for a tumor protein, comprising contacting T cells with at least one component selected from the group consisting of:
(a) polypeptides according to claim 2;
(b) polynucleotides according to claim 1; and
(c) antigen-presenting cells that express a polynucleotide according to claim 1, under conditions and for a time sufficient to permit the stimulation and/or expansion of T cells.

10. An isolated T cell population, comprising T cells prepared according to the method of claim 9.

11. A composition comprising a first component selected from the group consisting of physiologically acceptable carriers and immunostimulants, and a second component selected from the group consisting of:
(a) polypeptides according to claim 2;
(b) polynucleotides according to claim 1;
(c) antibodies according to claim 5;
(d) fusion proteins according to claim 7;
(e) T cell populations according to claim 10; and
(f) antigen presenting cells that express a polypeptide according to claim 2.

12. A method for stimulating an immune response in a patient, comprising administering to the patient a composition of claim 11.

13. A method for the treatment of a cancer in a patient, comprising administering to the patient a composition of claim 11.

14. A method for determining the presence of a cancer in a patient, comprising the steps of:
(a) obtaining a biological sample from the patient;
(b) contacting the biological sample with an oligonucleotide according to claim 8;
(c) detecting in the sample an amount of a polynucleotide that hybridizes to the oligonucleotide; and
(d) compare the amount of polynucleotide that hybridizes to the oligonucleotide to a predetermined cut-off value, and therefrom determining the presence of the cancer in the patient.

15. A diagnostic kit comprising at least one oligonucleotide according to claim 8.

16. A diagnostic kit comprising at least one antibody according to claim 5 and a detection reagent, wherein the detection reagent comprises a reporter group.

17. A method for inhibiting the development of a cancer in a patient, comprising the steps of:
(a) incubating CD4+ and/or CD8+ T cells isolated from a patient with at least one component selected from the group consisting of: (i) polypeptides according to claim 2; (ii) polynucleotides according to claim 1; and (iii) antigen presenting cells that express a polypeptide of claim 2, such that T cell proliferate;
(b) administering to the patient an effective amount of the proliferated T cells, and thereby inhibiting the development of a cancer in the patient.
